SEQUENCE LISTING (80 sequences)

SEQ ID NO: 1 - protein, 492 aa, synthetic construct
  REGION 1..492: note = CD16 (F176V)-P2A-NKG2D transgene
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDKGSGATNFSLLKQAGDVEENPGPMGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 2 - protein, 254 aa, synthetic construct
  REGION 1..254: note = CD16 F176V variant protein
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 3 - protein, 22 aa, synthetic construct
  REGION 1..22: note = P2A peptide
GSGATNFSLLKQAGDVEENPGP

SEQ ID NO: 4 - protein, 216 aa, synthetic construct
  REGION 1..216: note = NKG2D wildtype protein
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 5 - protein, 254 aa, synthetic construct
  REGION 1..254: note = CD16 wildtype protein
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 6 - DNA, 1479 bp, synthetic construct
  misc_feature 1..1479: note = CD16 (F176V)-P2A-NKG2D polynucleotide
  source 1..1479: mol_type = other DNA
atgtggcaactgttgctgcctacagctctgctgctgctggtgtctgccggcatgagaacagaggatctgcctaaggccgtggtgttcctggaacctcagtggtacagagtgctggaaaaggacagcgtgaccctgaagtgccagggcgcctattctcccgaggacaatagcacccagtggttccacaacgagagcctgatcagcagccaggccagcagctactttatcgatgccgccaccgtggacgacagcggcgagtacagatgccagaccaatctgagcaccctgagcgaccctgtgcagctggaagtgcacattggatggctgctgcttcaggcccctagatgggtgttcaaagaagaggaccccatccacctgagatgccactcttggaagaacacagccctgcacaaagtgacctacctgcagaacggcaagggcagaaagtacttccaccacaacagcgacttctacatccccaaggccacactgaaggactccggctcctacttctgtagaggcctcgtgggcagcaagaacgtgtccagcgagacagtgaacatcaccatcacacagggcctcgccgtgtctaccatcagcagctttttcccacctggctatcaggtgtccttctgcctggtcatggtgctgctgttcgccgtggataccggcctgtacttcagcgtcaagaccaacatccggtccagcaccagagactggaaggaccacaagttcaagtggcggaaggaccctcaggacaaaggcagcggcgccaccaatttcagcctgctgaaacaggctggcgacgtggaagagaaccctggacctatgggctggatccggggcagaagaagcagacacagctgggagatgagcgagttccacaattacaacctggacctgaagaagtccgacttcagcacccggtggcagaaacagagatgccccgtggtcaagagcaagtgcagagagaacgctagccccttcttcttctgttgctttatcgccgtggccatgggcatccgcttcatcatcatggtcacaatttggagcgccgtgttcctgaactccctgttcaatcaagaggtgcagatccctctgaccgagagctactgtggcccctgtcctaagaactggatctgctacaagaacaactgctaccagttcttcgacgagagcaagaattggtacgagagccaggcctcctgcatgagccagaatgcttccctgctgaaggtgtacagcaaagaggaccaggatctgctgaagctggtcaagtcctaccactggatgggcctcgtgcacatccctaccaatggctcttggcagtgggaggacggcagcattctgagccctaacctgctgaccatcatcgagatgcagaagggcgactgcgccctgtacgccagctctttcaagggctacatcgagaactgcagcacccctaacacctacatctgtatgcagcggaccgtgtga

SEQ ID NO: 7 - DNA, 762 bp, synthetic construct
  misc_feature 1..762: note = CD16 F176V polynucleotide
  source 1..762: mol_type = other DNA
atgtggcaactgttgctgcctacagctctgctgctgctggtgtctgccggcatgagaacagaggatctgcctaaggccgtggtgttcctggaacctcagtggtacagagtgctggaaaaggacagcgtgaccctgaagtgccagggcgcctattctcccgaggacaatagcacccagtggttccacaacgagagcctgatcagcagccaggccagcagctactttatcgatgccgccaccgtggacgacagcggcgagtacagatgccagaccaatctgagcaccctgagcgaccctgtgcagctggaagtgcacattggatggctgctgcttcaggcccctagatgggtgttcaaagaagaggaccccatccacctgagatgccactcttggaagaacacagccctgcacaaagtgacctacctgcagaacggcaagggcagaaagtacttccaccacaacagcgacttctacatccccaaggccacactgaaggactccggctcctacttctgtagaggcctcgtgggcagcaagaacgtgtccagcgagacagtgaacatcaccatcacacagggcctcgccgtgtctaccatcagcagctttttcccacctggctatcaggtgtccttctgcctggtcatggtgctgctgttcgccgtggataccggcctgtacttcagcgtcaagaccaacatccggtccagcaccagagactggaaggaccacaagttcaagtggcggaaggaccctcaggacaaa

SEQ ID NO: 8 - DNA, 66 bp, synthetic construct
  misc_feature 1..66: note = P2A polynucleotide
  source 1..66: mol_type = other DNA
ggcagcggcgccaccaatttcagcctgctgaaacaggctggcgacgtggaagagaaccctggacct

SEQ ID NO: 9 - DNA, 651 bp, synthetic construct
  misc_feature 1..651: note = NKG2D polynucleotide
  source 1..651: mol_type = other DNA
atgggctggatccggggcagaagaagcagacacagctgggagatgagcgagttccacaattacaacctggacctgaagaagtccgacttcagcacccggtggcagaaacagagatgccccgtggtcaagagcaagtgcagagagaacgctagccccttcttcttctgttgctttatcgccgtggccatgggcatccgcttcatcatcatggtcacaatttggagcgccgtgttcctgaactccctgttcaatcaagaggtgcagatccctctgaccgagagctactgtggcccctgtcctaagaactggatctgctacaagaacaactgctaccagttcttcgacgagagcaagaattggtacgagagccaggcctcctgcatgagccagaatgcttccctgctgaaggtgtacagcaaagaggaccaggatctgctgaagctggtcaagtcctaccactggatgggcctcgtgcacatccctaccaatggctcttggcagtgggaggacggcagcattctgagccctaacctgctgaccatcatcgagatgcagaagggcgactgcgccctgtacgccagctctttcaagggctacatcgagaactgcagcacccctaacacctacatctgtatgcagcggaccgtgtga

SEQ ID NO: 10 - DNA, 20 bp, synthetic construct
  misc_feature 1..20: note = CD70 gRNA target sequence
  source 1..20: mol_type = other DNA
gtcccattggtcgcgggctt

SEQ ID NO: 11 - DNA, 973 bp, synthetic construct
  misc_feature 1..973: note = CD70 exon 1 left homology arm
  source 1..973: mol_type = other DNA
aaaaataaaaaataaaaatattaacttaatttaactttaaacaaaaaagcaggtggtctccaagaatgcaggagataactgaccgggtgcagtgtctcatgcctttaatcccagcactttggaaggccaaggcgggtggatcacccaaggtcaggagttcaagtccagcctggccaacatggtgaaaccccatctctactaaaaatacaaaaaattagccaggcatggtggcgcgcgcatgttactcccagctactcgcgaggctcagacaggagaatcgcttgaacccaggagatcgaggttgcggcgagctgagatggcgccactgcactccagcctgggtgacagagggagacctccgtctcaaaaacaaaacaaatcaaaaaaatgcaggagaggggtacacgaatatttggggagcacccccaattcttggatgtctgctgtatccccagtgcacagcacaatctaatccctaataaatgtgcagtggaggtttgttgaataaatgaatgggccccagaagaatgaggtggagaggggaataggaagattgaatgtctcctgcctgaaggtcgggcggggaggggttgggggcaggcaactctgaggctcacccggggccactgcctgcatcctggcaactgcctccacccactttaggatcttcagactggcagcggttggagggaatttcccctcgccaattgctcaagtccctcccctcgaccggccggacatccccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggt

SEQ ID NO: 12 - DNA, 1000 bp, synthetic construct
  misc_feature 1..1000: note = CD70 exon 1 right homology arm
  source 1..1000: mol_type = other DNA
cgcgggcttggtgatctgcctcgtggtgtgcatccagcgcttcgcacaggctcagcagcagctgccgctcgagtcacttggggtgagttgagatggaaaagttgggaagaaaacatagagaggcgcgtgaccgaaaagacagaatgagatgggtacaaagaggccagagaggaagatctggtagggcagagacagagaccagaacagggaggcgaggcggggaccaggctgcccggtgtaggggctacgagacaggcagccctgccaggaggtacagggagatcccgggatgggaaaggtaggcacacatggaaatggaagatgactcggctctggtgttcccccggcaggctgactcagaggctgctgggggcttcacaaggctgggcgtgggggcttcctggggcctcctaggacgggatggccccagccactcgctccgggtgggggaggggtccctttggggaccgcgccgggcgcctttgcagcgtagagagtccgctgcgcgcggtgctctcgcgcccagtgacatccaggaaaacgattcgggaaacgaagaagttcttttgaaggtctcgacttcacgttccccgctggttcagacctgcttcctctttaagaagtcttaagagtaaaaaaaaataaaatgaaataaaatcaccagtgcgcgccgtgggatgagaggtggaaaggaggatggacagagaaaagagagctcctggcacaggggacacatagaacctctctgcttacgtccgtgccctgttttctggtcttttcttccagtgggacgtagctgagctgcagctgaatcacacaggtaacacgggggacgtggagggacggggagaagaagaggcacagagagagaaggaaggagaggtagaaagacaagtggggagagacagagagaaagagacacagacagagacggagggagagagggagggagagatagggagggaaacggagagggggagacagagagaagacagagagg

SEQ ID NO: 13 - DNA, 7301 bp, synthetic construct
  misc_feature 1..7301: note = CD16 transgene
  source 1..7301: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggataaaaataaaaaataaaaatattaacttaatttaactttaaacaaaaaagcaggtggtcttgctttggtcccattggtccaagaatgcaggagataactgaccgggtgcagtgtctcatgcctttaatcccagcactttggaaggccaaggcgggtggatcacccaaggtcaggagttcaagtccagcctggccaacatggtgaaaccccatctctactaaaaatacaaaaaattagccaggcatggtggcgcgcgcatgttactcccagctactcgcgaggctcagacaggagaatcgcttgaacccaggagatcgaggttgcggcgagctgagatggcgccactgcactccagcctgggtgacagagggagacctccgtctcaaaaacaaaacaaatcaaaaaaatgcaggagaggggtacacgaatatttggggagcacccccaattcttggatgtctgctgtatccccagtgcacagcacaatctaatccctaataaatgtgcagtggaggtttgttgaataaatgaatgggccccagaagaatgaggtggagaggggaataggaagattgaatgtctcctgcctgaaggtcgggcggggaggggttgggggcaggcaactctgaggctcacccggggccactgcctgcatcctggcaactgcctccacccactttaggatcttcagactggcagcggttggagggaatttcccctcgccaattgctcaagtccctcccctcgaccggccggacatccccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtggcctccaacgcgttaattcgaaattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaagtcgaccgccaccatgtggcagctgctgctgcccaccgccctgctgctgctggtgagcgccggcatgagaaccgaggacctgcccaaggccgtggtgttcctggagccccagtggtacagagtgctggagaaggacagcgtgaccctgaagtgccagggcgcctacagccccgaggacaacagcacccagtggttccacaacgagagcctgatcagcagccaggccagcagctacttcatcgacgccgccaccgtggacgacagcggcgagtacagatgccagaccaacctgagcaccctgagcgaccccgtgcagctggaggtgcacatcggctggctgctgctgcaggcccccagatgggtgttcaaggaggaggaccccatccacctgagatgccacagctggaagaacaccgccctgcacaaggtgacctacctgcagaacggcaagggcagaaagtacttccaccacaacagcgacttctacatccccaaggccaccctgaaggacagcggcagctacttctgcagaggcctggtgggcagcaagaacgtgagcagcgagaccgtgaacatcaccatcacccagggcctggccgtgagcaccatcagcagcttcttcccccccggctaccaggtgagcttctgcctggtgatggtgctgctgttcgccgtggacaccggcctgtacttcagcgtgaagaccaacatcagaagcagcaccagagactggaaggaccacaagttcaagtggagaaaggacccccaggacaagtgataaggcatgcaagcttaacttgtttattgcagcttataatggttacaaataaagcaatagcatcacaaatttcacaaataaagcatttttttcactgcattctagttgtggtttgtccaaactcatcaatgtatcttatcatgtctgggtaccgaattccgcgggcttggtgatctgcctcgtggtgtgcatccagcgcttcgcacaggctcagcagcagctgccgctcgagtcacttggggtgagttgagatggaaaagttgggaagaaaacatagagaggcgcgtgaccgaaaagacagaatgagatgggtacaaagaggccagagaggaagatctggtagggcagagacagagaccagaacagggaggcgaggcggggaccaggctgcccggtgtaggggctacgagacaggcagccctgccaggaggtacagggagatcccgggatgggaaaggtaggcacacatggaaatggaagatgactcggctctggtgttcccccggcaggctgactcagaggctgctgggggcttcacaaggctgggcgtgggggcttcctggggcctcctaggacgggatggccccagccactcgctccgggtgggggaggggtccctttggggaccgcgccgggcgcctttgcagcgtagagagtccgctgcgcgcggtgctctcgcgcccagtgacatccaggaaaacgattcgggaaacgaagaagttcttttgaaggtctcgacttcacgttccccgctggttcagacctgcttcctctttaagaagtcttaagagtaaaaaaaaataaaatgaaataaaatcaccagtgcgcgccgtgggatgagaggtggaaaggaggatggacagagaaaagagagctcctggcacaggggacacatagaacctctctgcttacgtccgtgccctgttttctggtcttttcttccagtgggacgtagctgagctgcagctgaatcacacaggtaacacgggggacgtggagggacggggagaagaagaggcacagagagagaaggaaggagaggtagaaagacaagtggggagagacagagagaaagagacacagacagagacggagggagagagggagggagagatagggagggaaacggagagggggagacagagagaagacagagaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 14 - protein, 335 aa, synthetic construct
  REGION 1..335: note = HLA-E protein
HSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 15 - protein, 312 aa, synthetic construct
  REGION 1..312: note = HLA-G protein
HSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 16 - protein, 162 aa, synthetic construct
  REGION 1..162: note = IL-15 protein
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 17 - protein, 423 aa, synthetic construct
  REGION 1..423: note = IL-15/IL-15Ra fusion protein
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGGGGSGGGGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 18 - protein, 237 aa, synthetic construct
  REGION 1..237: note = IL-15Ra protein
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 19 - protein, 504 aa, synthetic construct
  REGION 1..504: note = signal peptide-B2M-linker-HLA-E protein
MVVMAPRTLFLLLSGALTLTETWAVMAPRTLILGGGGSGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 20 - protein, 471 aa, synthetic construct
  REGION 1..471: note = signal peptide-B2M-linker-HLA-G protein
MVVMAPRTLFLLLSGALTLTETWARIIPRHLQLGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 21 - DNA, 1515 bp, synthetic construct
  misc_feature 1..1515: note = signal peptide-B2M-linker-HLA-E transgene
  source 1..1515: mol_type = other DNA
atggtggtcatggcccctagaacactgttcctgctgctgtctggcgccctgacactgacagagacatgggccgtgatggcccccagaaccctgatcctgggcggcggtggttcaggcggaggaggttcaggaggagggggtagtggaggtggtggttctatccagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgacactgagccagcctaagatcgtgaagtgggatcgcgatatgggcggaggcggatctggtggcggaggaagtggcggcggaggatctggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 22 - DNA, 1422 bp, synthetic construct
  misc_feature 1..1422: note = signal peptide-B2M-linker-HLA-G transgene
  source 1..1422: mol_type = other DNA
gccaccatggtggtcatggcgccccgaaccctcttcctgctgctctcgggggccctgaccctgaccgagacctgggcgcggatcattccccgacatctgcaactgggaggcggcggttcaggagggggcggatcgatccaacgcacccccaagatccaggtctactccagacacccggccgaaaacggaaagtcgaacttcctgaactgctatgtgtcaggattccacccgtccgacatcgaggtggacctcctgaagaacggcgaacgcattgagaaggtcgagcactccgatctgtcgttctccaaggactggtccttctaccttctctactataccgaattcaccccgaccgagaaggacgaatacgcctgccgggtcaaccacgtgaccctgagccagccaaagatcgtgaaatgggaccgcgatatgggaggaggaggttccggcggaggaggaagcggaggcggaggttccggctcccactccatgaggtatttcagcgccgccgtgtcccggcctggccgcggagagcctcgcttcatcgccatgggatacgtggacgacacccagttcgtcagattcgacagcgacagcgcctgtcctcggatggaacctagagcaccttgggtcgagcaagagggccctgagtactgggaagaagagacacggaacaccaaggctcacgcccagaccgacagaatgaacctgcagaccctgcggggctactacaatcagtctgaggccagcagccatactctgcagtggatgatcggctgcgatctgggctctgatggcagactgctgagaggctacgagcagtacgcctacgacggcaaggattatctggccctgaacgaggacctgcggtcttggacagctgccgatacagccgctcagatcagcaagagaaagtgcgaggccgccaatgtggccgaacagagaagggcttacctggaaggcacctgtgtggaatggctgcacagatacctggaaaacggcaaagagatgctgcagcgggccgatcctcctaagacacatgtgacccaccatcctgtgttcgactacgaggccacactgagatgttgggccctgggcttttaccctgccgagatcatcctgacctggcagcgagatggcgaggatcagacccaggatgtggaactggtggaaaccagacctgccggcgacggcacctttcagaaatgggctgctgtggtggtgcccagcggagaggaacagagatacacctgtcacgtgcagcacgagggactgcctgaacctctgatgctgagatggaagcagagcagcctgcctacaatccccatcatgggaatcgtggccggactggtggttctggccgctgttgttacaggtgctgcagtggctgccgtgctgtggcggaagaaaagcagcgactga

SEQ ID NO: 23 - protein, 376 aa, synthetic construct
  REGION 1..376: note = HSV thymidine kinase protein
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 24 - protein, 741 aa, synthetic construct
  REGION 1..741: note = PSMA variant protein
MWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 25 - protein, 1135 aa, synthetic construct
  REGION 1..1135: note = HSV-TK-PSMA variant protein
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSTSGSGKPGSGEGSTKGMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 26 - DNA, 3408 bp, synthetic construct
  misc_feature 1..3408: note = HSV-TK-PSMA variant transgene
  source 1..3408: mol_type = other DNA
atggcctcatatccctgccaccagcatgcgagtgcgtttgatcaagctgcacggtcaagaggacattccaataggcgaactgcactgagacctaggcgacaacaagaagcaacggaggtccgccttgagcagaagatgcctactcttcttagggtttacatcgatggtccgcacggaatgggtaagaccacgactactcaactgcttgtcgcattgggctccagggatgacatagtttacgttccggaacctatgacttattggcaggtgttgggggcatccgaaaccatagctaatatttataccacgcaacaccgcttggaccaaggagagatctctgcgggagatgcagcggtggtaatgaccagcgcacaaatcaccatggggatgccgtatgccgtaacagacgcggtgctggccccccatatcggtggtgaagccggttcaagtcatgcgcctccgccggcgctgacattgatctttgatcggcatcctattgcggcactcctgtgttatccagccgcacgctatctgatgggaagcatgaccccacaagcggttctggcctttgtggcattgatcccaccaacattgcctggaactaatattgtactgggcgctctccccgaggatcgacatattgataggctcgcgaaacgccaacgccctggtgaaaggcttgatttggccatgctcgcggctatacgccgcgtctatgggttgctcgccaatactgtgagatacctgcagggcggggggtcatggcgcgaggattggggtcagttgtcaggcacggcagtgcccccacagggggcggagcctcagtcaaatgcaggcccaagaccgcatattggtgatactctcttcacgttgtttcgggcgccagaattgcttgcccccaacggtgatctttataacgtcttcgcatgggccctcgatgtcttggccaagaggctgcgaccgatgcacgtttttattctcgactatgaccagtcacctgcgggatgcagggatgcgttgctccagttgacaagtggcatggtgcagacacatgtgacgacacccggatcaatacctacgatatgtgatctcgcaaggacctttgcgagggaaatgggggaagctaatggctcaacttctggatccggaaagcccggtagcggcgaaggttctacaaagggtatgtggaacctgttggcaagacgcccccgctggctctgtgccggtgctctggtactggcggggggattttttctcttgggatttctttttgggtggttcataaaaagttctaatgaggccacaaatatcactccgaaacacaatatgaaggcatttctggacgagctcaaagcggaaaatattaagaaattcctgtataactttactcagatacctcatctggctggcaccgagcaaaacttccagttggctaagcagattcaatcacagtggaaagagttcggtctcgatagcgttgaattggcacactacgatgtccttcttagctatcctaataaaacacatccgaactacataagtatcattaatgaggacggcaacgaaattttcaacacctctctttttgaacctcctccgccgggatatgaaaacgtgagcgatatcgttccccccttttccgcgttctcaccacagggaatgccggagggtgaccttgtttatgtaaattatgccagaacggaagatttctttaaacttgaacgcgatatgaaaatcaactgctctggtaagattgttattgcccgatacgggaaggtattcaggggtaacaaagtgaaaaacgctcagctggcgggtgccaaaggagtgatcctttattctgaccctgcggattatttcgcaccaggcgtaaaatcatatcctgacggttggaaccttcctggaggtggagtacagcgcggaaatatattgaatcttaacggagccggtgacccactgactcctggataccccgcaaacgagtatgcctatcgacgcggcattgccgaagcggtgggactgccctcaatacctgtacatcctattggatactatgatgctcagaaacttttggagaagatgggtggaagtgccccgcctgatagttcctggagaggctcccttaaggttccatataacgtaggtccagggtttacgggcaacttttcaacacaaaaggtaaagatgcatatacattcaactaacgaggtgacgaggatatacaatgtaatcggaactctgaggggagccgtagagcctgatcgatatgtcatcttggggggccacagggatagttgggtctttggtggaattgatcctcagtcaggtgcggctgtagtccacgagattgtccgctctttcggcacgctgaagaaggaggggtggagaccccgaaggactattttgtttgcctcttgggatgctgaagaatttggtctgctcggatcaacggagtgggctgaagagaattctaggttgttgcaagaacgcggtgtggcctacataaatgcggacagtagtatagaaggcaattacacacttcgagtggattgcaccccgcttatgtacagtctggtacataacctgacgaaggagcttaaatcacctgatgaaggattcgaggggaaatccctttacgaatcatggactaaaaagtcaccttcccctgaatttagtgggatgccgcgcataagtaaactcgggtccggaaacgacttcgaagttttcttccaacgattgggtatcgcctctggacgagcacggtacaccaaaaattgggaaacgaacaaattttccggatatcctctctaccactctgtctatgaaacctacgagctggtggaaaagttttacgatccgatgtttaagtaccatttgaccgtcgcccaggtgcggggaggaatggtctttgaattggcaaatagtatagtccttccttttgattgtcgagattatgccgtcgtccttaggaagtatgctgacaagatttattcaatatctatgaagcacccccaagaaatgaagacctactcagtgtcctttgactccctgttctccgctgtgaagaacttcactgagatcgcctctaagttctcagagcgactgcaagattttgacaagagtaaccccattgttcttaggatgatgaatgaccagctcatgtttttggagagagcatttattgatccgctgggccttcccgaccggccattttacagacacgttatctatgctccttcaagtcacaataaatatgcaggagaatcctttcctgggatctacgatgccctcttcgacatagaaagtaaggttgatccctccaaggcgtggggggaagttaaacgacagatatatgtcgctgcattcaccgtacaggccgccgcagagacacttagtgaggttgcgtga

SEQ ID NO: 27 - protein, 1139 aa, synthetic construct
  REGION 1..1139: note = HSV-TK-T2A-PSMA protein
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANSGSGEGRGSLLTCGDVEENPGPMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 28 - DNA, 3420 bp, synthetic construct
  misc_feature 1..3420: note = HSV-TK-T2A-PSMA transgene
  source 1..3420: mol_type = other DNA
atggcaagttacccttgccaccagcatgctagcgctttcgatcaagcggcccgcagtcggggccatagcaatagaaggaccgcattgcgcccgcgccgacagcaggaagctaccgaagtacgactggaacaaaaaatgcccactcttctgagagtatacatcgatgggccacacggcatgggcaaaacgacgactacccaactcttggtagctcttggtagccgggatgatatcgtatatgtgccagaacctatgacctattggcaggtcctcggcgccagtgaaaccatcgccaacatatatacgacgcaacataggcttgaccagggtgaaatctccgcaggtgacgcggcagtggtcatgactagcgcccaaatcacgatgggaatgccttatgcggtgactgacgcagtactcgctcctcacattggaggtgaagcggggagctcccacgcaccgccgcccgctcttacgctcattttcgatcgccaccctatagctgccctgctctgttatcccgcggccaggtacttgatggggtccatgaccccccaggcggtgctggccttcgttgcgttgataccgccaactctccccggcactaatattgttctcggagcacttccagaagacaggcatattgacaggttggctaagcgccaaaggcccggtgaacgactcgacctggctatgcttgctgccatccgccgcgtctatgggctgctggctaatacggtcaggtatcttcaaggcggcggatcttggagggaagattggggacagctcagtggaacggctgtacctccacaaggggccgaacctcagtcaaatgcaggtcctcgccctcacattggagatacactttttactcttttccgggctccagaactgctggcaccaaatggcgacctgtacaatgtttttgcctgggctctggatgttttggcaaaaaggcttcggccgatgcacgtatttattttggactatgatcagtcaccggctggttgtagagacgcattgcttcaacttacatccgggatggtacaaacgcatgtaacaaccccagggtcaattccaactatctgcgatctcgcccgcacattcgcaagagaaatgggtgaggctaactctggcagtggtgaaggccgcggatctctcctgacttgtggggatgtcgaagaaaacccgggacccatgtggaacctgttggcaagacgcccccgctggctctgtgccggtgctctggtactggcggggggattttttctcttgggatttctttttgggtggttcataaaaagttctaatgaggccacaaatatcactccgaaacacaatatgaaggcatttctggacgagctcaaagcggaaaatattaagaaattcctgtataactttactcagatacctcatctggctggcaccgagcaaaacttccagttggctaagcagattcaatcacagtggaaagagttcggtctcgatagcgttgaattggcacactacgatgtccttcttagctatcctaataaaacacatccgaactacataagtatcattaatgaggacggcaacgaaattttcaacacctctctttttgaacctcctccgccgggatatgaaaacgtgagcgatatcgttccccccttttccgcgttctcaccacagggaatgccggagggtgaccttgtttatgtaaattatgccagaacggaagatttctttaaacttgaacgcgatatgaaaatcaactgctctggtaagattgttattgcccgatacgggaaggtattcaggggtaacaaagtgaaaaacgctcagctggcgggtgccaaaggagtgatcctttattctgaccctgcggattatttcgcaccaggcgtaaaatcatatcctgacggttggaaccttcctggaggtggagtacagcgcggaaatatattgaatcttaacggagccggtgacccactgactcctggataccccgcaaacgagtatgcctatcgacgcggcattgccgaagcggtgggactgccctcaatacctgtacatcctattggatactatgatgctcagaaacttttggagaagatgggtggaagtgccccgcctgatagttcctggagaggctcccttaaggttccatataacgtaggtccagggtttacgggcaacttttcaacacaaaaggtaaagatgcatatacattcaactaacgaggtgacgaggatatacaatgtaatcggaactctgaggggagccgtagagcctgatcgatatgtcatcttggggggccacagggatagttgggtctttggtggaattgatcctcagtcaggtgcggctgtagtccacgagattgtccgctctttcggcacgctgaagaaggaggggtggagaccccgaaggactattttgtttgcctcttgggatgctgaagaatttggtctgctcggatcaacggagtgggctgaagagaattctaggttgttgcaagaacgcggtgtggcctacataaatgcggacagtagtatagaaggcaattacacacttcgagtggattgcaccccgcttatgtacagtctggtacataacctgacgaaggagcttaaatcacctgatgaaggattcgaggggaaatccctttacgaatcatggactaaaaagtcaccttcccctgaatttagtgggatgccgcgcataagtaaactcgggtccggaaacgacttcgaagttttcttccaacgattgggtatcgcctctggacgagcacggtacaccaaaaattgggaaacgaacaaattttccggatatcctctctaccactctgtctatgaaacctacgagctggtggaaaagttttacgatccgatgtttaagtaccatttgaccgtcgcccaggtgcggggaggaatggtctttgaattggcaaatagtatagtccttccttttgattgtcgagattatgccgtcgtccttaggaagtatgctgacaagatttattcaatatctatgaagcacccccaagaaatgaagacctactcagtgtcctttgactccctgttctccgctgtgaagaacttcactgagatcgcctctaagttctcagagcgactgcaagattttgacaagagtaaccccattgttcttaggatgatgaatgaccagctcatgtttttggagagagcatttattgatccgctgggccttcccgaccggccattttacagacacgttatctatgctccttcaagtcacaataaatatgcaggagaatcctttcctgggatctacgatgccctcttcgacatagaaagtaaggttgatccctccaaggcgtggggggaagttaaacgacagatatatgtcgctgcattcaccgtacaggccgccgcagagacacttagtgaggttgcgtga

SEQ ID NO: 29 - protein, 376 aa, synthetic construct
  REGION 1..376: note = HSV-TK A168H variant protein
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 30 - protein, 1138 aa, synthetic construct
  REGION 1..1138: note = HSV-TK-T2A-PSMA N9del protein
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGEGRGSLLTCGDVEENPGPMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 31 - protein, 1138 aa, synthetic construct
  REGION 1..1138: note = HSV A168H -TK-T2A-PSMA N9del protein
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGEGRGSLLTCGDVEENPGPMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 32 - DNA, 3382 bp, synthetic construct
  misc_feature 1..3382: note = HSV A168H -TK-T2A-PSMA N9del transgene
  source 1..3382: mol_type = other DNA
atggccagctatccttgtcaccagcacgccagcgcctttgatcaggccgcaagatctagaggccacagcaacagaagaacagccctgcggcctcggagacagcaagaggctacagaagttcggctggaacagaagatgcccacactgctgcgggtgtacatcgatggccctcacggcatgggcaagaccaccacaacacagctgctggtggccctgggcagcagagatgatatcgtgtacgtgcccgagcctatgacctactggcaggttctgggagccagcgagacaatcgccaacatctacaccacacagcaccggctggatcagggcgaaatttctgctggcgacgccgccgtggttatgacatctgcccagatcaccatgggcatgccttacgccgtgacagatgctgtgctggcccctcacattggcggagaagccggatcttctcatgcccctccaccagctctgaccctgatcttcgacagacaccctatcgcccatctgctgtgttatcctgccgccagatacctgatgggcagcatgacacctcaggccgtgctggctttcgtggccctgattcctcctacactgcccggcaccaatatcgtgctgggagccctgcctgaggaccggcacattgatagactggccaagagacagcggcctggcgagagactggatctggctatgctggccgccatcagaagagtgtacggcctgctggccaacaccgtgcggtatcttcaaggcggcggatcttggagagaggactggggacaactgagcggcacagcagttcctccacaaggcgctgagcctcagtctaacgctggacccagacctcacatcggcgacaccctgtttaccctgttcagagcccctgagctgctggctcctaacggcgacctgtacaacgtgttcgcctgggctcttgacgtgctggcaaaaagactgcggcccatgcacgtgttcatcctggactacgatcagtcccctgccggctgtagagatgctctgctgcagctgacaagcggcatggtgcagacccacgttacaacccctggcagcatccccaccatctgtgacctggccagaaccttcgccagagagatgggagaagccaacggcagcggcgaaggcagaggatctctgctgacatgtggcgacgtggaagagaaccccggacctatgtggaacctgctggctagacggcccagatggctttgtgctggtgctctggttctggctggcggctttttcctgctgggcttcctgtttggctggttcatcaagagcagcaacgaggccaccaacatcacccctaagcacaacatgaaggcctttctggacgagctgaaggccgagaatatcaagaagttcctctacaacttcacgcagatccctcacctggccggcaccgagcagaattttcagctggccaagcagatccagagccagtggaaagagttcggcctggactctgtggaactggcccactacgatgtgctgctgagctaccccaacaagacacaccccaactacatcagcatcatcaacgaggacggcaacgagatcttcaacaccagcctgttcgagcctccacctcctggctacgagaacgtgtccgatatcgtgcctccattcagcgctttcagccctcaagggatgcctgagggcgatctggtgtacgtgaactacgccagaaccgaggacttcttcaagctggaacgggacatgaagatcaactgctccggcaagatcgtgatcgcccgctacggcaaagtgttccggggcaacaaagtgaagaacgcccagctggcaggcgccaaaggcgtgatcctgtatagcgaccccgccgactattttgcccctggcgtgaagtcttaccccgacggctggaatcttcctggtggcggagtgcagagaggcaacatcctgaaccttaacggcgcaggcgaccctctgacacctggctatcctgccaatgagtacgcctacagacggggaattgccgaggctgtgggactgccttctatccctgtgcaccccatcggctactacgacgcccagaaactgctggaaaagatgggcggaagcgcccctcctgactctagttggagaggctctctgaaggtgccctacaatgtcggcccaggcttcaccggcaacttcagcacccagaaagtgaaaatgcacatccacagcaccaacgaagtgacccggatctacaacgtgatcggcacactgagaggcgccgtggaacccgacagatatgtgatcctcggcggccacagagatagctgggtgttcggaggaatcgaccctcagtctggtgccgctgtggtgcacgaaatcgtgcggtcttttggcaccctgaagaaagaaggctggcgccccagacggaccatcctgtttgcctcttgggacgccgaggaattcggactgctgggatctacagagtgggccgaagagaacagcagactgctgcaagaaagaggcgtggcctacatcaacgccgacagcagcatcgagggcaactacaccctgagagtggactgcacccctctgatgtacagcctggtgcacaacctgaccaaagagctgaagtcccctgacgagggctttgagggcaagagcctgtacgagagctggaccaagaagtccccatctcctgagttcagcggcatgccccggatctctaagctcggctctggcaacgacttcgaggtgttcttccagcggctgggaatcgcttctggcagagccagatacaccaagaactgggagacaaacaagttctccggctatcccctgtaccacagcgtgtacgagacatacgagctggtcgagaagttctacgaccccatgttcaagtaccacctgacagtggcccaagtgcgcggaggcatggtgttcgaactggccaatagcatcgtgctgcccttcgactgcagagactatgccgtggtgctgcggaagtacgccgataagatctacagcatcagcatgaagcacccgcaagagatgaagacctacagcgtgtccttcgatagcctgttcagcgccgtgaagaacttcaccgagatcgccagcaagttcagcgagcggctgcaggacttcgacaagagcaacccaatcgtgctgaggatgatgaacgaccagctgatgttcctggaacgcgccttcatcgacccactgggcttgcccgatagacccttctaccggcacgtgatctatgcccctagcagccacaacaaatacgccggcgagagcttccccggcatctacgatgccctgttcgacatcgagagcaaggtggacccatctaaggcctggggcgaagtgaagcggcagatctacgtggccgcattcacagtgc

SEQ ID NO: 33 - protein, 543 aa, synthetic construct
  REGION 1..543: note = IL-12 protein
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSTSGSGKPGSGEGSTKGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 34 - DNA, 1629 bp, synthetic construct
  misc_feature 1..1629: note = IL-12 transgene
  source 1..1629: mol_type = other DNA
atgtgccatcaacaattggttatatcatggttctctctcgtgtttctggcatcaccacttgttgcaatctgggaactgaaaaaagatgtatatgtagtcgaattggactggtatccagatgcgcccggcgagatggttgttctgacttgtgacacacctgaggaagacggcattacgtggacgctggatcaatcttccgaggtcctcggatctggtaaaacccttacaattcaagttaaggaatttggtgatgcaggacaatacacctgccacaagggtggtgaagtactgtcccattccctcctgttgctgcacaagaaggaggacggaatatggagtacagacatcctgaaggaccagaaagagcccaaaaacaagacgtttttgagatgcgaggcaaagaactacagtggtcggttcacgtgctggtggttgactaccatttcaacagatctgacattttcagtcaagtcaagtagagggtcttcagacccgcaaggtgttacatgtggcgctgcaacgctctccgcagagagggttaggggagacaacaaggagtatgagtatagtgtcgagtgtcaagaagacagtgcctgtcccgcggcagaggaatccctcccaattgaggtcatggtagatgctgttcacaagctcaagtatgaaaattatacttcaagtttttttatcagagacattatcaagccggatccccctaaaaatctccagcttaagcccctcaaaaatagtcggcaggtcgaagtgagctgggaatatcccgacacgtggtctaccccgcactcatacttcagtctgactttttgcgtccaagtacaaggaaagtccaagagagaaaaaaaggatagagtgtttaccgacaagactagcgcgacggttatttgtcggaagaacgcgagcatttcagttcgagcacaggacaggtattattcatcttcatggtcagaatgggcttcagttccgtgcagcggctctacttccggctcaggtaagccgggctctggagagggtagcactaagggcaggaacttgcctgtcgccaccccggacccaggcatgttcccttgtttgcatcacagtcagaatttgctgcgagcggtctccaacatgcttcaaaaagctcggcaaaccctcgaattctatccgtgcactagcgaggaaatagaccacgaagacataactaaggacaaaacaagtactgtggaagcctgcctgcccctcgaacttacgaaaaacgagagctgcctgaatagccgagaaacctctttcattactaatgggagctgtctggcgagtcggaagacctcatttatgatggcgctttgtctttcctcaatttacgaagacctcaagatgtaccaggttgaatttaaaacgatgaacgccaagttgcttatggatccgaagcggcagatatttcttgatcagaatatgcttgctgttatagatgagttgatgcaggctctcaacttcaacagcgagactgttccacagaagtcatctctggaagaacccgatttctacaagaccaagataaaactctgtattctcttgcatgcttttcggattcgggcagtgacgatcgacagggttatgtcttaccttaatgccagt

SEQ ID NO: 35 - protein, 484 aa, synthetic construct
  REGION 1..484: note = DLL4-Fc fusion 4 protein
SSVFQLQLQEFINERGVLASGRPCEPGCRTFFRVCLKHFQAVVSPGPCTFGTVSTPVLGTNSFAVRDDSSGGGRNPLQLPLNFTWPGTFSLIIEAWHAPGDDLRPEALPPDALISKFAIQGSLAVGQNWLLDEQTSTLTRLRYSYRVICSDNYYGDNCSRLCKKRNDYFGHYVCQPDGNPSCLPGWTGEYCQQPICLSGCHEQNGYCSKPAECLCRPGWQGRLCNECIPHNGCRHGTCSTPWQCTCDEGWGGLFCDQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 484 aa, synthetic construct; identical to SEQ ID NO: 35
  REGION 1..484: note = DLL4-Fc fusion 5 protein

SEQ ID NO: 37 - protein, 725 aa, synthetic construct
  REGION 1..725: note = DLL4-Fc fusion 6 protein
SSVFQLQLQEFINERGVLASGRPCEPGCRTFFRVCLKHFQAVVSPGPCTFGTVSTPVLGTNSFAVRDDSSGGGRNPLQLPLNFTWPGTFSLIIEAWHAPGDDLRPEALPPDALISKFAIQGSLAVGQNWLLDEQTSTLTRLRYSYRVICSDNYYGDNCSRLCKKRNDYFGHYVCQPDGNPSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 1272 bp, synthetic construct
  misc_feature 1..1272: note = IL-15/IL-15Ra fusion transgene
  source 1..1272: mol_type = other DNA
atgcgcatctctaagcctcatctgcgatccatcagtatccaatgttacctgtgtctgctgcttaatagtcattttctgactgaagccggaatccatgtctttatcctgggctgcttcagtgcgggattgcccaaaactgaagccaactgggttaacgtgatctctgatttgaagaaaatagaagaccttatacaaagtatgcatattgatgccacgctctataccgaatcagacgttcatccctcatgcaaagtcacagctatgaagtgttttctgctggagctgcaggttattagtctggagagtggagacgccagcatccacgatacggtcgagaatctcattatcttggcaaataattcactctcttcaaacggaaacgtgaccgaatcagggtgcaaggagtgcgaggaactcgaagaaaagaatatcaaagagttcctgcagagcttcgtgcacatcgtgcaaatgttcatcaatacctctgggggaggtggttcagggggtggaggttctggtggggggggctcaggaggagggggtagcggcggtggtggaagcatcacgtgtcccccaccgatgtctgtagaacacgctgacatctgggtcaaatcttactctctgtactctcgcgaacgctacatttgcaactcagggtttaaacggaaagccggaacctctagtctgacggagtgtgtgctgaataaggcgacaaacgtagcgcactggaccacccctagtctcaagtgcataagggacccagctttggtacaccagaggcctgcaccgccctccaccgtcactacggctggcgtaacaccgcaaccggagtctctgagtcctagcggaaaagagcccgccgcaagtagcccttcaagtaacaacaccgccgcgacgacggcggctattgtgcccggtagtcaattgatgccttccaagtctccgtccacaggcacgacagagataagttcacacgaaagtagccatgggactccttcacagacgaccgcaaagaattgggagttgacggcttcagcctcacaccagcccccgggcgtgtatccgcagggacatagcgataccacggtagccataagtactagtacagtattgctgtgcggactctctgccgtcagtcttcttgcttgttatttgaaatccagacagacgccaccgctggcctccgtggaaatggaagcgatggaagccctccctgtaacgtggggaacaagcagtcgagacgaagatctggagaactgctcacatcacttg

SEQ ID NO: 39 - DNA, 9584 bp, synthetic construct
  misc_feature 1..9584: note = CD16-2A-NKG2D targeting construct
  source 1..9584: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggataaaaataaaaaataaaaatattaacttaatttaactttaaacaaaaaagcaggtggtctccaagaatgcaggagataactgaccgggtgcagtgtctcatgcctttaatcccagcactttggaaggccaaggcgggtggatcacccaaggtcaggagttcaagtccagcctggccaacatggtgaaaccccatctctactaaaaatacaaaaaattagccaggcatggtggcgcgcgcatgttactcccagctactcgcgaggctcagacaggagaatcgcttgaacccaggagatcgaggttgcggcgagctgagatggcgccactgcactccagcctgggtgacagagggagacctccgtctcaaaaacaaaacaaatcaaaaaaatgcaggagaggggtacacgaatatttggggagcacccccaattcttggatgtctgctgtatccccagtgcacagcacaatctaatccctaataaatgtgcagtggaggtttgttgaataaatgaatgggccccagaagaatgaggtggagaggggaataggaagattgaatgtctcctgcctgaaggtcgggcggggaggggttgggggcaggcaactctgaggctcacccggggccactgcctgcatcctggcaactgcctccacccactttaggatcttcagactggcagcggttggagggaatttcccctcgccaattgctcaagtccctcccctcgaccggccggacatccccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtggcctccaacgcgttaattcgaaattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaagtcgacgccaccatgtggcaactgttgctgcctacagctctgctgctgctggtgtctgccggcatgagaacagaggatctgcctaaggccgtggtgttcctggaacctcagtggtacagagtgctggaaaaggacagcgtgaccctgaagtgccagggcgcctattctcccgaggacaatagcacccagtggttccacaacgagagcctgatcagcagccaggccagcagctactttatcgatgccgccaccgtggacgacagcggcgagtacagatgccagaccaatctgagcaccctgagcgaccctgtgcagctggaagtgcacattggatggctgctgcttcaggcccctagatgggtgttcaaagaagaggaccccatccacctgagatgccactcttggaagaacacagccctgcacaaagtgacctacctgcagaacggcaagggcagaaagtacttccaccacaacagcgacttctacatccccaaggccacactgaaggactccggctcctacttctgtagaggcctcgtgggcagcaagaacgtgtccagcgagacagtgaacatcaccatcacacagggcctcgccgtgtctaccatcagcagctttttcccacctggctatcaggtgtccttctgcctggtcatggtgctgctgttcgccgtggataccggcctgtacttcagcgtcaagaccaacatccggtccagcaccagagactggaaggaccacaagttcaagtggcggaaggaccctcaggacaaaggcagcggcgccaccaatttcagcctgctgaaacaggctggcgacgtggaagagaaccctggacctatgggctggatccggggcagaagaagcagacacagctgggagatgagcgagttccacaattacaacctggacctgaagaagtccgacttcagcacccggtggcagaaacagagatgccccgtggtcaagagcaagtgcagagagaacgctagccccttcttcttctgttgctttatcgccgtggccatgggcatccgcttcatcatcatggtcacaatttggagcgccgtgttcctgaactccctgttcaatcaagaggtgcagatccctctgaccgagagctactgtggcccctgtcctaagaactggatctgctacaagaacaactgctaccagttcttcgacgagagcaagaattggtacgagagccaggcctcctgcatgagccagaatgcttccctgctgaaggtgtacagcaaagaggaccaggatctgctgaagctggtcaagtcctaccactggatgggcctcgtgcacatccctaccaatggctcttggcagtgggaggacggcagcattctgagccctaacctgctgaccatcatcgagatgcagaagggcgactgcgccctgtacgccagctctttcaagggctacatcgagaactgcagcacccctaacacctacatctgtatgcagcggaccgtgtgataataatgaaacttgtttattgcagcttataatggttacaaataaagcaatagcatcacaaatttcacaaataaagcatttttttcactgcattctagttgtggtttgtccaaactcatcaatgtatcttatcatgtctgggtaccgaattccgcgggcttggtgatctgcctcgtggtgtgcatccagcgcttcgcacaggctcagcagcagctgccgctcgagtcacttggggtgagttgagatggaaaagttgggaagaaaacatagagaggcgcgtgaccgaaaagacagaatgagatgggtacaaagaggccagagaggaagatctggtagggcagagacagagaccagaacagggaggcgaggcggggaccaggctgcccggtgtaggggctacgagacaggcagccctgccaggaggtacagggagatcccgggatgggaaaggtaggcacacatggaaatggaagatgactcggctctggtgttcccccggcaggctgactcagaggctgctgggggcttcacaaggctgggcgtgggggcttcctggggcctcctaggacgggatggccccagccactcgctccgggtgggggaggggtccctttggggaccgcgccgggcgcctttgcagcgtagagagtccgctgcgcgcggtgctctcgcgcccagtgacatccaggaaaacgattcgggaaacgaagaagttcttttgaaggtctcgacttcacgttccccgctggttcagacctgcttcctctttaagaagtcttaagagtaaaaaaaaataaaatgaaataaaatcaccagtgcgcgccgtgggatgagaggtggaaaggaggatggacagagaaaagagagctcctggcacaggggacacatagaacctctctgcttacgtccgtgccctgttttctggtcttttcttccagtgggacgtagctgagctgcagctgaatcacacaggtaacacgggggacgtggagggacggggagaagaagaggcacagagagagaaggaaggagaggtagaaagacaagtggggagagacagagagaaagagacacagacagagacggagggagagagggagggagagatagggagggaaacggagagggggagacagagagaagacagagaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgggcctccaaggccttaattcgaaactagtaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagatgtacgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgaacgctagcgctgccggttaggggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 40 - protein, 18 aa, synthetic construct
  REGION 1..18: note = Whitlow Linker
GSTSGSGKPGSGEGSTKG

SEQ ID NO: 41 - protein, 5 aa, synthetic construct
  REGION 1..5: note = GGGGS or a multimer thereof
GGGGS

SEQ ID NO: 42 - protein, 15 aa, synthetic construct
  REGION 1..15: note = (G4S)3
GGGGSGGGGSGGGGS

SEQ ID NO: 43 - protein, 20 aa, synthetic construct
  REGION 1..20: note = Linker 3
GGSEGKSSGSGSESKSTGGS

SEQ ID NO: 44 - protein, 8 aa, synthetic construct
  REGION 1..8: note = Linker 4
GGGSGGGS

SEQ ID NO: 45 - protein, 12 aa, synthetic construct
  REGION 1..12: note = Linker 4
GGGSGGGSGGGS

SEQ ID NO: 46 - protein, 16 aa, synthetic construct
  REGION 1..16: note = Linker 6
GGGSGGGSGGGSGGGS

SEQ ID NO: 47 - protein, 20 aa, synthetic construct
  REGION 1..20: note = Linker 7
GGGSGGGSGGGSGGGSGGGS

SEQ ID NO: 48 - protein, 20 aa, synthetic construct
  REGION 1..20: note = Linker 8
GGGGSGGGGSGGGGSGGGGS

SEQ ID NO: 49 - protein, 25 aa, synthetic construct
  REGION 1..25: note = Linker 9
GGGGSGGGGSGGGGSGGGGSGGGGS

SEQ ID NO: 50 - protein, 14 aa, synthetic construct
  REGION 1..14: note = Linker 10
IRPRAIGGSKPRVA

SEQ ID NO: 51 - protein, 15 aa, synthetic construct
  REGION 1..15: note = Linker 11
GKGGSGKGGSGKGGS

SEQ ID NO: 52 - protein, 15 aa, synthetic construct
  REGION 1..15: note = Linker 12
GGKGSGGKGSGGKGS

SEQ ID NO: 53 - protein, 15 aa, synthetic construct
  REGION 1..15: note = Linker 13
GGGKSGGGKSGGGKS

SEQ ID NO: 54 - protein, 15 aa, synthetic construct
  REGION 1..15: note = Linker 14
GKGKSGKGKSGKGKS

SEQ ID NO: 55 - protein, 15 aa, synthetic construct
  REGION 1..15: note = Linker 15
GGGKSGGKGSGKGGS

SEQ ID NO: 56 - protein, 15 aa, synthetic construct
  REGION 1..15: note = Linker 16
GKPGSGKPGSGKPGS

SEQ ID NO: 57 - protein, 20 aa, synthetic construct
  REGION 1..20: note = Linker 17
GKPGSGKPGSGKPGSGKPGS

SEQ ID NO: 58 - protein, 20 aa, synthetic construct
  REGION 1..20: note = Linker 18
GKGKSGKGKSGKGKSGKGKS

SEQ ID NO: 59 - protein, 14 aa, synthetic construct
  REGION 1..14: note = Linker 19
STAGDTHLGGEDFD

SEQ ID NO: 60 - protein, 15 aa, synthetic construct
  REGION 1..15: note = Linker 20
GEGGSGEGGSGEGGS

SEQ ID NO: 61 - protein, 15 aa, synthetic construct
  REGION 1..15: note = Linker 21
GGEGSGGEGSGGEGS

SEQ ID NO: 62 - protein, 15 aa, synthetic construct
  REGION 1..15: note = Linker 22
GEGESGEGESGEGES

SEQ ID NO: 63 - protein, 15 aa, synthetic construct
  REGION 1..15: note = Linker 23
GGGESGGEGSGEGGS

SEQ ID NO: 64 - protein, 20 aa, synthetic construct
  REGION 1..20: note = Linker 24
GEGESGEGESGEGESGEGES

SEQ ID NO: 65 - protein, 18 aa, synthetic construct
  REGION 1..18: note = Linker 25
GSTSGSGKPGSGEGSTKG

SEQ ID NO: 66 - protein, 19 aa, synthetic construct
  REGION 1..19: note = Linker 26
PRGASKSGSASQTGSAPGS

SEQ ID NO: 67 - protein, 19 aa, synthetic construct
  REGION 1..19: note = Linker 27
GTAAAGAGAAGGAAAGAAG

SEQ ID NO: 68 - protein, 19 aa, synthetic construct
  REGION 1..19: note = Linker 28
GTSGSSGSGSGGSGSGGGG

SEQ ID NO: 69 - protein, 20 aa, synthetic construct
  REGION 1..20: note = Linker 29
GKPGSGKPGSGKPGSGKPGS

SEQ ID NO: 70 - protein, 4 aa, synthetic construct
  REGION 1..4: note = Linker 30
GSGS

SEQ ID NO: 71 - protein, 10 aa, synthetic construct
  REGION 1..10: note = Linker 31
APAPAPAPAP

SEQ ID NO: 72 - protein, 20 aa, synthetic construct
  REGION 1..20: note = Linker 32
APAPAPAPAPAPAPAPAPAP

SEQ ID NO: 73 - protein, 32 aa, synthetic construct
  REGION 1..32: note = Linker 33
AEAAAKEAAAKEAAAAKEAAAAKEAAAAKAAA

SEQ ID NO: 74 - protein, 15 aa, synthetic construct
  REGION 1..15: note = hinge domain
EPKSCDKTHTCPPCP

SEQ ID NO: 75 - protein, 12 aa, synthetic construct
  REGION 1..12: note = hinge domain
ERKCCVECPPCP

SEQ ID NO: 76 - protein, 62 aa, synthetic construct
  REGION 1..32: note = hinge domain
ELKTPLGDTTHTCPRCPEPKSCDTPPPCPRCPEPKSCDTPPPCPRCPEPKSCDTPPPCPRCP

SEQ ID NO: 77 - protein, 12 aa, synthetic construct
  REGION 1..12: note = hinge domain
ESKYGPPCPSCP

SEQ ID NO: 78 - DNA, 140 bp, synthetic construct
  misc_feature 1..140: note = Top DNA strand of exon 1 of human CD70 gene
  source 1..140: mol_type = other DNA
ccgcggcgatgccggaggagggttcgggctgctcggtgcggcgcaggccctatgggtgcgtcctgcgggctgctttggtcccattggtcgcgggcttggtgatctgcctcgtggtgtgcatccagcgcttcgcacaggct

SEQ ID NO: 79 - DNA, 140 bp, synthetic construct
  misc_feature 1..140: note = Bottom DNA strand of exon 1 of human CD70 gene
  source 1..140: mol_type = other DNA
ggcgccgctacggcctcctcccaagcccgacgagccacgccgcgtccgggatacccacgcaggacgcccgacgaaaccagggtaaccagcgcccgaaccactagacggagcaccacacgtaggtcgcgaagcgtgtccga

SEQ ID NO: 80 - protein, 44 aa, synthetic construct
  REGION 1..44: note = CD70 protein encoded by exon 1
MPEEGSGCSVRRRPYGCVLRAALVPLVAGLVICLVVCIQRFAQA

(19) United States
(12) Patent Application Publication
(10) Pub. No.: US 2026/0256839 A1
(43) Pub. Date: Sep. 3, 2026

(54) GENETICALLY ENGINEERED CELLS EXPRESSING CD16 VARIANTS AND NKG2D AND USES THEREOF

(71) Applicant: Century Therapeutics, Inc., Philadelphia, PA (US)

(72) Inventors: Michael NASO, Philadelphia, PA (US); Buddha GURUNG, Philadelphia, PA (US); Barry MORSE, Philadelphia, PA (US); Luis BORGES, Philadelphia, PA (US)

(21) Appl. No.: 18/871,540

(22) PCT Filed: Jun. 7, 2023
(86) PCT No.: PCT/US2023/068079

§ 371 Date: Dec. 4, 2024

Related U.S. Application Data
(60) Provisional application No. 63/350,298, filed on Jun. 8, 2022.

(51) Int. Cl. A61K 35/17; A61K 40/11; A61K 40/31; A61K 40/42; C07K 14/54; C07K 14/705; C07K 14/715; C07K 16/28; C12N 5/074

(52) U.S. Cl. CPC A61K 35/17; A61K 40/11; A61K 40/31; A61K 40/4211; C07K 14/5443; C07K 14/7051; C07K 14/70521; C07K 14/70535; C07K 14/70539; C07K 14/7056; C07K 14/7155; C07K 16/2803; C12N 5/0696; A61K 2239/13; A61K 2239/17; A61K 2239/21; C07K 2317/53; C07K 2317/569; C07K 2317/622; C07K 2317/732; C07K 2319/02; C07K 2319/03; C07K 2319/50; C12N 2510/00

(57) ABSTRACT

Disclosed herein are engineered iPSCs and cells derived therefrom that express CD16 variants and NKG2D. Also, the engineered iPSCs and cells derived therefrom can express a chimeric antigen receptor. Additionally, provided herein are polynucleotide constructs and methods of using thereof that are useful for expressing CD16 variants and NKG2D in such cells.

### CROSS-REFERENCE

This application claims the benefit of U.S. Provisional Application No. 63/350,298 filed Jun. 8, 2022, which is incorporated herein by reference in its entirety.

### SEQUENCE LISTING INCORPORATION

This instant application contains a Sequence Listing, which has been submitted electronically in XML format in accordance to the WIPO Standard ST.26 and is hereby incorporated by reference in its entirety. The XML copy, created on Jun. 7, 2023 is named “SL.xml” and is 150,741 bytes in size.

### BACKGROUND

Cancer continues to be a significant global health issue despite substantial research efforts and scientific advances towards treating the disease. Cancer immunotherapies are desirable because they are highly specific and can facilitate removal of the cancer cells by using the patient's immune system. Research is ongoing to develop effective immune cell-based therapies for treating cancer.

Activated NK cells can kill target cells such as cancer cells by means similar to cytotoxic T cells (i.e., via cytolytic granules that contain perforin and granzymes as well as via death receptor pathways). Activated NK cells also secrete inflammatory cytokines such as IFN-7 and chemokines that promote the recruitment of other leukocytes to target tissues such as cancer tissues.

Sources of immune cells include those that have been differentiated from induced pluripotent stem cells (iPSCs). These cells can be modified to be allogeneic. There remains an unmet need for therapeutically sufficient and functional engineered allogeneic iPSC-derived therapies for treating cancer. Further, in engineering cell therapies, it is desirable to minimize the number of genetic edits that need to be made to the cells. Thus, there is a need for engineered cell therapies with multiple functionalities that can be engineered with a minimum number of edits.

### BRIEF SUMMARY

In one aspect, described is an induced pluripotent stem cell (iPSC) or a derivative cell thereof comprising an exogenous polynucleotide encoding a CD16 protein and an NKG2D protein, wherein the CD16 protein and the NKG2D protein are operably linked by an autoprotease peptide.

In some embodiments, the CD16 protein is a CD16 variant protein. In some embodiments, the CD16 variant is a high affinity CD16 variant. In many embodiments, the CD16 variant is a non-cleavable CD16 variant. In various embodiments, the CD16 variant comprises one or more amino acid substitutions selected from the group consisting of F158V, F176V, S197P, D205A, S219A, T220A, and any combination thereof. In some embodiments, the CD16 variant comprises an amino acid sequence having at least 90% sequence identity to any one of SEQ ID NOS: 2 and 5.

In some embodiments, the NKG2D protein is a wildtype NKG2D protein. In many embodiments, the NKG2D protein comprises an amino acid sequence having at least 90% sequence identity to SEQ ID NO:4.

In some embodiments, the autoprotease peptide is selected from the group consisting of a porcine tesehovirus-1 2A (P2A) peptide, a foot-and-mouth disease virus 2A (F2A) peptide, an Equine Rhinitis A Virus (ERAV) 2A (E2A) peptide, a Thosea asigna virus 2A (T2A) peptide, a cytoplasmic polyhedrosis virus 2A (BmCPV2A) peptide, and a Flacherie Virus 2A (BmIFV2A) peptide. In many embodiments, the autoprotease peptide is a P2A peptide comprising an amino acid sequence having at least 90% sequence identity to SEQ ID NO:3.

In some embodiments, the exogenous polynucleotide encoding the CD16 protein and the NKG2D protein comprises a nucleic acid sequence having at least 90% sequence identity to SEQ ID NO:6.

In some embodiments, the exogenous polynucleotide is integrated into a gene locus selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus.

In some embodiments, the exogenous polynucleotide is integrated into a gene locus selected from the group consisting of an AAVS1 locus, a B2M locus, a CIITA locus, a CD70 locus, a CLYBL locus, an NKG2A locus, an NKG2D locus, a TAP1 locus, a TAP2 locus, a TAPBP locus, a TRAC locus, a TRBC1 locus, a RFXANK locus, a RFX5 locus, and a RFXAP locus, thereby disrupting expression of the gene.

In some embodiments, the exogenous polynucleotide is integrated into a gene locus selected from the group consisting of an AAVS1 locus, a B2M locus, a CIITA locus, a CD70 locus, a CLYBL locus, an NKG2A locus and a TRAC locus, thereby disrupting expression of the gene.

In some embodiments, the disruption of the gene comprises an elimination of or reduced expression of the gene. In some embodiments, the integration into the gene locus is generated by targeted genome editing.

In some embodiments, the targeted genome editing comprises using a method selected from the group consisting of a CRISPR method, a zinc finger nuclease method, a TALEN method, a homing nuclease method, a homology recombination method, and any functional variation thereof.

In some embodiments, the method further comprises a disruption of one or more genes selected from the group consisting of an AAVS1 gene, a B2M gene, a CIITA gene, a CD70 gene, a CLYBL gene, an NKG2A gene, an NKG2D gene, a TAP1 gene, a TAP2 gene, a TAPBP gene, a TRAC gene, a TRBC1 gene, a RFXANK gene, a RFX5 gene, a RFXAP gene, and any combination thereof.

In some embodiments, the disruption is of the B2M gene and the CIITA gene.

T In some embodiments, the iPSC or the derivative cell thereof of claim 18 or 19, wherein the disruption of the one or more genes comprises an elimination of or reduced expression of the one or more genes. In many embodiments, the disruption of the one or more genes is generated by targeted genome editing.

In some embodiments, the targeted genome editing comprises using a method selected from the group consisting of a CRISPR method, a zinc finger nuclease method, a TALEN method, a homing nuclease method, a homology recombination method, and any functional variation thereof.

In some embodiments, the iPSC or the derivative cell further comprises a second exogenous polynucleotide encoding an IL-15 protein. In some embodiments, the IL-15 protein comprise an amino acid sequence having at least 90% sequence identity to SEQ ID NO:16.

In some embodiments, the iPSC or the derivative cell further comprises a second exogenous polynucleotide encoding a fusion polypeptide comprising an IL-15 and an IL-15 receptor alpha (IL-15Rα). In some embodiments, the fusion polypeptide comprises an amino acid sequence having at least 90% sequence identity to SEQ ID NO:17. In some embodiments, the fusion polypeptide comprises the amino acid sequence of SEQ ID NO:17.

In some embodiments, the iPSC or the derivative cell further comprises a third exogenous polynucleotide encoding a human leukocyte antigen E (HLA-E) protein. In some embodiments, the HLA-E comprises an amino acid sequence having at least 90% sequence identity to SEQ ID NO:14.

In some embodiments, the iPSC or the derivative cell further comprises a fourth exogenous polynucleotide encoding a human leukocyte antigen G (HLA-G) protein. In some embodiments, the HLA-G comprises an amino acid sequence having at least 90% sequence identity to SEQ ID NO:15.

In some embodiments, the HLA-E protein and the HLA-G protein are operably linked by a second autoprotease peptide. In some embodiments, the second autoprotease peptide is selected from the group consisting of a P2A peptide, an F2A peptide, an E2A peptide, a T2A peptide, a BmCPV2A peptide and a BmIFV2A peptide.

In some embodiments, the second, third, and/or fourth exogenous polynucleotides are integrated into one or more gene loci selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus, and any combination thereof.

In some embodiments, the second, third, and/or fourth exogenous polynucleotides are integrated into one or more gene loci selected from the group consisting of an AAVS1 locus, a B2M locus, a CIITA locus, a CD70 locus, a CLYBL locus, an NKG2A locus, an NKG2D locus, a TAP1 locus, a TAP2 locus, a TAPBP locus, a TRAC locus, a TRBC1 locus, a RFXANK locus, a RFX5 locus, a RFXAP locus, and any combination thereof.

In some embodiments, the second, third, and/or fourth exogenous polynucleotides are integrated into one or more gene loci selected from the group consisting of an AAVS1 locus, a B2M locus, a CIITA locus, a CD70 locus, a CLYBL locus, an NKG2A locus, an NKG2D locus, a TAP1 locus, a TAP2 locus, a TAPBP locus, a TRAC locus, a TRBC1 locus, a RFXANK locus, a RFX5 locus, a RFXAP locus, and any combination thereof, thereby disrupting the one or more genes. In some embodiments, the disruption in the one or more genes comprises an elimination or reduced expression of the one or more genes.

In some embodiments, the second, third, and/or fourth exogenous polynucleotides are integrated into one or more gene loci selected from the group consisting of an AAVS1 locus, a B2M locus, a CIITA locus, a CD70 locus, a CLYBL locus, an NKG2A locus, a TRAC locus, and any combination thereof, thereby disrupting the one or more genes. In some embodiments, the disruption in the one or more genes comprises an elimination or reduced expression of the one or more genes.

In some embodiments, the iPSC is reprogrammed from whole peripheral blood mononuclear cells (PBMCs). In some embodiments, the iPSC is derived from a reprogrammed NK or T cell.

In some embodiments, the iPSC or the derivative cell thereof further comprises a fifth exogenous polynucleotide encoding a chimeric antigen receptor (CAR) that binds a target antigen.

In some embodiments, the target antigen is selected from the group consisting of 17-1A antigen, A3, A33 antigen, AFP, B7H4, Ba 733, BCMA, BrE3 antigen, CA125, CA9 (CAIX), CD1, CD1a, CD3, CD5, CD15, CD16, CD19, CD20, CD21, CD22, CD22, CD23, CD25, CD30, CD33, CD33, CD38, CD45, CD70, CD74, CD79, CD79a, CD80, CD123, CD133, CD138, CEACAM5, CEACAM6, CLDN18.2, CLL1, cMET, colon-specific antigen-p (CSAp), ED-B fibronectin, EGFR, EGFRvIII, EGP-1, EGP-2, EpCAM, EphA1, EphA2, EphA3, EphA4, EphA5, EphA6, EphA7, EphA8, EphA10, EphB1, EphB2, EphB3, EphB4, EphB6, FGFR1, FGFR3, Flt-1, Flt-3, FOLR1, FOLR2, FOLR3, FSHR, GD2, GPC-3, GPRC5D, HCG, a HCG subunit, HER2, HIF-I, HLA-DR, Ia, IGF-I, IL13Rα2, IL-2, IL-6, IL-8, KC4 antigen, KS-1 antigen, KS1-4 antigen, Le-Y, MAGE, MET, MIF, MSLN, MUC1, MUC2, MUC3, MUC4, MUC16, NCA66, NCA90, NCA95, Nectin-4, p53, PAP, PDGFRA, PLGF, PSA, PSMA, ROBO1, RS5, S100, SLAM F7, SLITRK6, TAC, TAG-72, tenascin-C, tenascin-R, tenascin-W, tenascin-X, Thomson-Friedenreich antigen, Tn antigen, TRAILR1, TRAILR2, TRAILR3, TRAILR4, VEGF, a tumor necrosis antigen, an angiogenesis antigen, and an oncogene antigen. In some embodiments, the CAR comprises an antigen-binding domain selected from the group consisting of any provided in Tables 1, 2 and 3.

In some embodiments, the CAR comprises: (i) a signal peptide; (ii) an extracellular domain comprising a binding domain that specifically binds the target antigen; (iii) a hinge region; (iv) a transmembrane domain, (v) an intracellular signaling domain; and (vi) one or more co-stimulatory domains. In some embodiments, the signal peptide of a CAR comprises a GMCSFR signal peptide. In some embodiments, the extracellular domain of a CAR comprises an single chain Fv (scFv) or a VHH domain that specifically binds the target antigen. In some embodiments, the hinge region of a CAR comprises a CD28 hinge region. In some embodiments, the transmembrane domain of a CAR comprises a CD28 transmembrane domain. In some embodiments, the intracellular signaling domain of a CAR comprises a CD3ζ intracellular domain. In some embodiments, the one or more co-stimulatory domains of a CAR comprise a CD28 signaling domain.

In some embodiments, the derivative cell is an NK cell or a T cell. In other embodiments, the derivative cell is an NK cell. In many embodiments, the derivative cell is a T cell. In some embodiments, the derivative cell is a CD34+ hematopoietic progenitor cell.

In some embodiments, provided is a composition comprising a population of any one of the iPSCs or the derivative cells thereof described herein.

In another aspect, provided is an engineered cell comprising: (i) a first exogenous polynucleotide encoding a CD16 protein and an NKG2D protein, wherein the CD16 protein and the NKG2D protein are operably linked by an autoprotease peptide; (ii) a second exogenous polynucleotide encoding an exogenous polypeptide comprising an IL-15 protein; and (iii) optionally, a third exogenous polynucleotide encoding a human leukocyte antigen E (HLA-E) and/or a fourth exogenous polynucleotide encoding a human leukocyte antigen G (HLA-G).

In some embodiments, the engineered cell further comprises a fifth polynucleotide encoding a combined artificial cell death/reporter system polypeptide comprising an intracellular domain having a herpes simplex virus thymidine kinase (HSV-TK) and a linker, a transmembrane region, and an extracellular domain comprising a prostate-specific membrane antigen (PSMA) extracellular domain or fragment thereof.

In some embodiments, the HSV-TK comprises an amino acid sequence having at least 90%, 91%, 92%, 93%, 94%, 95%, 96%, 97%, 98%, 99% or 100% sequence identity to SEQ ID NO:23 or 29.

In some embodiments, the combined artificial cell death/reporter system polypeptide comprises the HSV-TK fused to a truncated variant PSMA polypeptide via the linker.

In some embodiments, the truncated variant PSMA polypeptide comprises an amino acid sequence having at least 90%, 91%, 92%, 93%, 94%, 95%, 96%, 97%, 98%, 99% or 100% sequence identity to SEQ ID NO:24.

In some embodiments, the linker comprises an autoprotease peptide sequence selected from the group consisting of P2A peptide sequence, T2A peptide sequence, E2A peptide sequence, and F2A peptide sequence. In certain embodiments, the linker is selected from any one of the group consisting of those set forth in Table 4.

In some embodiments, the artificial cell death/reporter system polypeptide comprises an amino acid sequence having at least 90%, 91%, 92%, 93%, 94%, 95%, 96%, 97%, 98%, 99% or 100% sequence identity to a sequence selected from the group consisting of SEQ ID NO:25.

In some embodiments, the artificial cell death/reporter system polypeptide comprises an amino acid sequence having at least 90%, 91%, 92%, 93%, 94%, 95%, 96%, 97%, 98%, 99% or 100% sequence identity to a sequence selected from the group consisting of SEQ ID NOS:27, 30 and 31.

In some embodiments, the artificial cell death/reporter system polypeptide comprises nucleic acid sequence having at least 90%, 91%, 92%, 93%, 94%, 95%, 96%, 97%, 98%, 99% or 100% sequence identity to a sequence selected from the group consisting of SEQ ID NOS:26, 28 and 32.

In another aspect, provided is an engineered cell comprising: (i) a first exogenous polynucleotide encoding a CD16 protein and an NKG2D protein, wherein the CD16 protein and the NKG2D protein are operably linked by an autoprotease peptide; (ii) a second exogenous polynucleotide encoding a fusion polypeptide comprising an IL-15 protein and an IL-15 receptor alpha (IL-15Rα) protein; and (iii) optionally, a third exogenous polynucleotide encoding a human leukocyte antigen E (HLA-E) protein and/or a fourth exogenous polynucleotide encoding a human leukocyte antigen G (HLA-G) protein.

is an engineered induced pluripotent stem cell (iPSC), an engineered natural killer (NK) cell or an engineered T cell.

In some embodiments, the first exogenous polynucleotide comprises the nucleic acid sequence of SEQ ID NO:6.

In some embodiments, the second exogenous polynucleotide comprises the nucleic acid sequence encoding an IL-15/IL-15Rα fusion protein of SEQ ID NO:17. In some embodiments, the second exogenous polynucleotide comprises the nucleic acid sequence of SEQ ID NO:38. In some embodiments, the second exogenous polynucleotide comprises the nucleic acid sequence having at least 90%, 91%, 92%, 93%, 94%, 95%, 96%, 97%, 98%, 99% or 100% sequence identity to SEQ ID NO:38. In some embodiments, the IL-15 protein comprises an amino acid sequence of SEQ ID NO: 16.

In some embodiments, the third exogenous polynucleotide comprises the nucleic acid sequence of SEQ ID NO:21 and the fourth exogenous polynucleotide comprises the nucleic acid sequence of SEQ ID NO:22.

In some embodiments, the HLA-E protein and HLA-G protein are linked by an autoprotease peptide. In some embodiments, the HLA-E protein of the engineered cell comprises the amino acid sequence of SEQ ID NO:19. In some embodiments, the HLA-G protein of the engineered cell comprises the amino acid sequence of SEQ ID NO:20. Described herein is a nucleic acid sequence of SEQ ID NO:21 which encodes the amino acid sequence of SEQ ID NO:19. Described herein is a nucleic acid sequence of SEQ ID NO:22 which encodes the amino acid sequence of SEQ ID NO:20.

In some embodiments, the engineered cell further comprises disruption of the B2M and CIITA genes. In some embodiments, the disruption of the B2M and CIITA genes is generated by targeted genome editing. In some embodiments, the targeted genome editing comprises using a method selected from the group consisting of a CRISPR method, a zinc finger nuclease method, a TALEN method, a homing nuclease method, a homology recombination method, and any functional variation thereof.

In some embodiments, the first exogenous polynucleotide is integrated into a gene locus selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus.

In some embodiments, the second exogenous polynucleotide is integrated into a gene locus selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, a collagen locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus.

In some embodiments, the third exogenous polynucleotide is integrated into a gene locus selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, a collagen locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus.

In some embodiments, the fourth exogenous polynucleotide is integrated into a gene locus selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, a collagen locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus.

In some embodiments, the first exogenous polynucleotide and either the second, third or fourth exogenous polynucleotides are integrated into the B2M gene locus and the CIITA gene locus, thereby disrupting the B2M and CIITA genes. In certain embodiments, the first exogenous polynucleotide is integrated into the CD70 locus and the second exogenous polynucleotide is integrated into the B2M gene locus, thereby disrupting the CD70 and B2M genes. In various embodiments, the first exogenous polynucleotide is integrated into the CD70 locus and the second exogenous polynucleotide is integrated into the CIITA gene locus, thereby disrupting the CD70 and CIITA genes.

In some embodiments, the first exogenous polynucleotide is integrated into the CD70 locus and the third or fourth exogenous polynucleotide is integrated into the B2M gene locus, thereby disrupting the CD70 and B2M genes.

In many embodiments, the first exogenous polynucleotide is integrated into the CD70 locus and the third or fourth exogenous polynucleotide is integrated into the CIITA gene locus, thereby disrupting the CD70 and CIITA genes. In various embodiments, the integration into the CD70 locus is into exon 1 of the CD70 gene.

In some embodiments, the integration into the gene locus is generated by targeted genome editing. In many embodiments, the targeted genome editing comprises using a method selected from the group consisting of a CRISPR method, a zinc finger nuclease method, a TALEN method, a homing nuclease method, a homology recombination method, and any functional variation thereof.

In some embodiments, the engineered cell further comprises a fifth exogenous polynucleotide encoding a chimeric antigen receptor (CAR) that binds a target antigen. In many embodiments, the target antigen is selected from the group consisting of 17-1A antigen, A3, A33 antigen, AFP, B7H4, Ba 733, BCMA, BrE3 antigen, CA125, CA9 (CAIX), CD1, CD1a, CD3, CD5, CD15, CD16, CD19, CD20, CD21, CD22, CD22, CD23, CD25, CD30, CD33, CD33, CD38, CD45, CD70, CD74, CD79, CD79a, CD80, CD123, CD133, CD138, CEACAM5, CEACAM6, CLDN18.2, CLL1, cMET, colon-specific antigen-p (CSAp), ED-B fibronectin, EGFR, EGFRvIII, EGP-1, EGP-2, EpCAM, EphA1, EphA2, EphA3, EphA4, EphA5, EphA6, EphA7, EphA8, EphA10, EphB1, EphB2, EphB3, EphB4, EphB6, FGFR1, FGFR3, Flt-1, Flt-3, FOLR1, FOLR2, FOLR3, FSHR, GD2, GPC-3, GPRC5D, HCG, a HCG subunit, HER2, HIF-I, HLA-DR, Ia, IGF-I, IL13Rα2, IL-2, IL-6, IL-8, KC4 antigen, KS-1 antigen, KS1-4 antigen, Le-Y, MAGE, MET, MIF, MSLN, MUC1, MUC2, MUC3, MUC4, MUC16, NCA66, NCA90, NCA95, Nectin-4, p53, PAP, PDGFRA, PLGF, PSA, PSMA, ROBO1, RS5, S100, SLAM F7, SLITRK6, TAC, TAG-72, tenascin-C, tenascin-R, tenascin-W, tenascin-X, Thomson-Friedenreich antigen, Tn antigen, TRAILR1, TRAILR2, TRAILR3, TRAILR4, VEGF, a tumor necrosis antigen, an angiogenesis antigen, and an oncogene antigen. In some embodiments, the CAR comprises an antigen-binding domain selected from the group consisting of any provided in Tables 1, 2 and 3.

In some embodiments, the CAR comprises: (i) a signal peptide; (ii) an extracellular domain comprising a binding domain that specifically binds the target antigen; (iii) a hinge region; (iv) a transmembrane domain, (v) an intracellular signaling domain; and (vi) one or more co-stimulatory domains.

In certain embodiments, the signal peptide of a CAR comprises a GMCSFR signal peptide. In many embodiments, the extracellular domain comprises an single chain Fv (scFv) or a VHH domain that specifically binds the target antigen. In various embodiments, the hinge region comprises a CD28 hinge region. In some embodiments, the transmembrane domain comprises a CD28 transmembrane domain. In certain embodiments, the intracellular signaling domain comprises a CD3ζ intracellular domain. In some embodiments, the one or more co-stimulatory domains comprise a CD28 signaling domain. In some embodiments, the engineered iPSC is differentiated into an engineered differentiated cell.

In some embodiments, the engineered iPSC is differentiated into an engineered NK cell. In some embodiments, the engineered iPSC is differentiated into an engineered T cell. In some embodiments, the engineered iPSC is differentiated into an engineered CD34+ hematopoietic progenitor cell.

Provided is a composition comprising a population of any one of the engineered iPSCs described herein. Provided is a composition comprising a population of any one of the engineered differentiated cells described herein. Also provided is a composition comprising a population of any one of the engineered NK cells described herein. And also provided is a composition comprising a population of any one of the engineered T cells described herein. Also provided is a composition comprising a population of any one of the engineered CD34+ hematopoietic progenitor cells described herein.

In one aspect, disclosed is a method of treating cancer in a subject in need thereof, comprising administering any of the derivative cells described, any of the engineered NK cells described, any of the engineered T cells described, any of the engineered CD34+ hematopoietic progenitor cells described, and any of the compositions described to the subject in need thereof.

In some embodiments, the cancer is selected from the group consisting of acute lymphocytic leukemia (ALL), acute myeloid leukemia (AML), adenomas, benign lesions, bladder cancers, bone cancers, breast cancers, cancers of the thyroid gland, carcinomas of the larynx, carcinomas of the lung, carcinomas of the mouth, carcinomas of the throat, cervical cancers, chronic lymphocytic leukemia (CLL), chronic myeloid leukemias (CML), cutaneous melanomas, endocrine cancers, endometrial cancers, gastrointestinal cancers, genitourinary cancers, glioblastomas, head and neck cancers, hematologic malignancy, hematopoietic cancers, Hodgkin's lymphoma, intraocular melanomas, leukemias, liver cancers, lymphomas, melanomas, myelomas, myeloproliferative disorders, nervous system cancers, non-Hodgkin's lymphoma, ovarian cancers, pancreatic cancers, papillomas, parathyroid gland cancers, prostate cancers, renal cell carcinomas, sarcomas, skin cancers, solid tissue carcinomas, squamous cell carcinomas, and uterine cancers.

In some aspects, provided is a method of differentiating the iPSC cell into an NK cell, comprising subjecting any one of the iPSC cells described to a differentiation protocol comprising culturing the cell in a medium comprising a recombinant human IL-12 protein for the final 24 hours of culturing under the differentiation protocol, thereby generating the NK cell. In some embodiments, the recombinant human IL-12 protein comprises an amino acid sequence having at least 90%, 91%, 92%, 93%, 94%, 95%, 96%, 97%, 98%, 99% or 100% sequence identity to SEQ ID NO:33.

In some aspects, provided is a method of differentiating the iPSC cell into a T cell, comprising subjecting any one of the iPSC cells described to a differentiation protocol comprising culturing the cell in a medium comprising a recombinant DLL4 variant polypeptide having an amino acid sequence selected from the group consisting of SEQ ID NOS:35-37, thereby generating the T cell. Provided is a recombinant DLL4 variant polypeptide having an amino acid sequence selected from the group consisting of SEQ ID NOS:35-37.

A method of differentiating the iPSC cell into a CD34+ hematopoietic progenitor cell, comprising subjecting any one of the iPSC cells described to a differentiation protocol comprising culturing the cell in a pre-selected medium, thereby generating the CD34+ hematopoietic progenitor cell.

In some aspects, provided is a polynucleotide encoding a CD16 protein and an NKG2D protein, wherein the CD16 protein and the NKG2D protein are operably linked by an autoprotease peptide. In many embodiments, the CD16 protein comprises an amino acid sequence having at least 90% sequence identity to SEQ ID NO:5. In some embodiments, the CD16 protein is encoded by a nucleic acid sequence having at least 90% sequence identity to SEQ ID NO:7. In various embodiments, the CD16 protein is a CD16 variant protein. In some embodiments, the CD16 variant protein comprises one or more amino acid substitutions selected from the group consisting of F158V, F176V, S197P, D205A, S219A, T220A, and any combination thereof.

In some embodiments, the CD16 variant comprises an amino acid sequence having at least 90% sequence identity to SEQ ID NO:2 or SEQ ID NO:5. In various embodiments, the CD16 variant is encoded by a nucleic acid sequence having at least 90% sequence identity, e.g., at least 90%, 91%, 92%, 93%, 94%, 95%, 96%, 97%, 98%, 99% or 100% sequence identity, to SEQ ID NO:7. In many embodiments, the NKG2D protein comprises an amino acid sequence having at least 90% sequence identity to SEQ ID NO:4. In some embodiments, the NKG2D protein is encoded by a nucleic acid sequence having at least 90% sequence identity, e.g., at least 90%, 91%, 92%, 93%, 94%, 95%, 96%, 97%, 98%, 99% or 100% sequence identity, to SEQ ID NO:9. In certain embodiments, the NKG2D protein is an NKG2D variant protein. In some embodiments, the NKG2D variant comprises an amino acid sequence having at least 90% sequence identity, e.g., at least 90%, 91%, 92%, 93%, 94%, 95%, 96%, 97%, 98%, 99% or 100% sequence identity, to SEQ ID NO: 4.

In some embodiments, the autoprotease peptide is selected from the group consisting of a porcine tesehovirus-1 2A (P2A) peptide, a foot-and-mouth disease virus (FMDV) 2A (F2A) peptide, an Equine Rhinitis A Virus (ERAV) 2A (E2A) peptide, a Thosea asigna virus 2A (T2A) peptide, a cytoplasmic polyhedrosis virus 2A (BmCPV2A) peptide, and a Flacherie Virus 2A (BmIFV2A) peptide. In some embodiments, the autoprotease peptide is a P2A peptide comprising an amino acid sequence having at least 90% sequence identity, e.g., at least 90%, 91%, 92%, 93%, 94%, 95%, 96%, 97%, 98%, 99% or 100% sequence identity, to SEQ ID NO:3. In some embodiments, the autoprotease peptide is a P2A peptide encoded by an nucleic acid sequence having at least 90% sequence identity, e.g., at least 90%, 91%, 92%, 93%, 94%, 95%, 96%, 97%, 98%, 99% or 100% sequence identity, to SEQ ID NO:8.

In some embodiments, the exogenous polynucleotide encoding the CD16 protein and the NKG2D protein comprises the nucleic acid sequence having at least 90% sequence identity, e.g., at least 90%, 91%, 92%, 93%, 94%, 95%, 96%, 97%, 98%, 99% or 100% sequence identity, to SEQ ID NO:6. In some embodiments, the exogenous polynucleotide encoding the CD16 protein and the NKG2D protein has the nucleic acid sequence of SEQ ID NO:6.

Provided is a vector comprising any of the polynucleotides described. In some embodiments, the vector comprises from 5′ to 3′: (i) a left homology sequence; (ii) a promoter; (iii) any of polynucleotides described; (iv) a terminator and/or a polyadenylation signal sequence; and (iv) a right homology sequence.

In some embodiments, the left homology sequence comprises a nucleic acid sequence having at least 90% sequence identity to SEQ ID NO:11. In some embodiments, the right homology sequence comprises a nucleic acid sequence having at least 90% sequence identity to SEQ ID NO:12.

In some embodiments, the vector comprises a nucleic acid sequence having at least 90% sequence identity, e.g., at least 90%, 91%, 92%, 93%, 94%, 95%, 96%, 97%, 98%, 99% or 100% sequence identity to SEQ ID NO:13. In some embodiments, the vector comprises the nucleic acid sequence of SEQ ID NO:13.

In some embodiments, the vector comprises a nucleic acid sequence having at least 90% sequence identity, e.g., at least 90%, 91%, 92%, 93%, 94%, 95%, 96%, 97%, 98%, 99% or 100% sequence identity, to SEQ ID NO:39. In some embodiments, the vector comprises the nucleic acid sequence of SEQ ID NO:39.

### BRIEF DESCRIPTION OF THE DRAWINGS

FIGS. 1A-1C depict an amino acid sequence and a nucleic acid sequence of the CD16 (F176V)-P2A-NKG2D transgene (SEQ ID NOS:1-9).

FIGS. 2A-2C depict sequences for CD70 targeted knock-in or knock-out in cells such as iPSC-derived natural killer cells (iNK cells). FIG. 2A provides a CD70 gRNA target sequence (SEQ ID NO:10). FIG. 2B provides a schematic diagram of exon 1 of the CD70 gene (SEQ ID NOS: 78-80). FIG. 2C depicts the nucleic acid sequence of an exemplary left homology arm (LHA) of the CD70 exon 1 targeting construct. FIG. 2D depicts the nucleic acid sequence of an exemplary right homology arm (RHA) of the CD70 exon 1 targeting construct (SEQ ID NOS:11-12).

FIG. 3 provides a schematic diagram of a targeting construct design including human CD16 transgene. The targeting construct includes a LHA targeting exon 1 of CD70, a constitutive promoter (CAG promoter), a KOZAK sequence, a human CD16 transgene, a SV40 terminator-poly adenylation signal, a RHA targeting exon 1 of CD70, and an selection marker (kanamycin-resistance marker).

FIGS. 4A-4G depict a nucleic acid sequence of the exemplary targeting construct sequence depicted in FIG. 3 and others. The CD70 exon homology arms are shown in bold, double underlined. The CAG promoter is shown in underlined. The human CD16 transgene is shown in bold. The SV40 terminator is shown in bold, underlined and the kanamycin resistance sequence is shown in double underlined. FIGS. 4D-4G present the nucleic acid sequence of an exemplary embodiment of a target sequence containing CD16-2A-NKG2D (SEQ ID NO:39).

FIGS. 5A-5N; FIGS. 5A-5D provide additional amino acid and nucleic acid sequences of IL-15-IL-15Rα, HLA-E and HLA-G fusions and components thereof (SEQ ID NOS:14-22 and 38). FIGS. 5E-5L provide additional amino acid and nucleic acid sequences of HSV-TK-PSMA fusions and components thereof (SEQ ID NOS:23-32). FIGS. 5L and 5M provide amino acid and nucleic acid sequences of IL-12 (SEQ ID NOS:33-34). FIGS. 5M and 5N provide amino acid sequences of various DLL4-Fc fusion proteins (SEQ ID NOS: 35-37).

FIG. 6 provides flow cytometry data detecting CD16 engineered into the CD70 locus using homology directed repair and CRISPR nuclease into iPSCs and differentiation into gamma/delta iT cells. iPSC1283 and iPSC1303 cells are iPSC cell lines expressing a CD16 transgene and a CAR. CD16 expression was measured on DO, D7, D14 and D21 of the cells undergoing differentiation into gamma/delta iT cells.

FIGS. 7A-7C show enhanced anti-tumor activity of iNK cells overexpressing NKG2D protein and enhanced antibody-dependent cellular cytotoxicity (ADCC) of iNK cells overexpressing high-affinity CD16. Engineered overexpression of NKG2D on iNK cells enhances anti-tumor activity is shown in FIGS. 7A-7B. iPSCs were engineered to constitutively express NKG2D. When iPSCs were differentiated into iNK cells, expression of NKG2D was quantified by flow cytometry and it is demonstrated that NKG2D expression was increased to 95.5% of iNK cells compared to 72.1% of non-engineered iNK cells (FIG. 7A). Non-engineered or NKG2D-engineered iNK cells were used in a killing assay with U87 glioblastoma cells that express stress ligands that trigger NKG2D activity. The NKG2D-engineered iNK cells more potently killed U87 cells. To confirm that enhanced killing was due to NKG2D expression, a neutralizing antibody against NKG2D was used in some conditions (to block the interaction of NKG2D with stress ligands on U87 cells). There was a marked reduction in U87 killing when the NKG2D neutralizing (blocking) antibody was included with the NKG2D-engineered iNK cells (FIG. 7B). Engineered overexpression of high-affinity CD16 on iNK cells enhances antibody-dependent cellular cytotoxicity (ADCC) (FIG. 7C). iPSCs were engineered to constitutively express one of two different naturally occurring variants of CD16. The iPSCs were then differentiated into iNK cells and used in a tumor killing assay where the targets were CD20+ lymphoblastic B cells. To trigger ADCC, anti-CD20 therapeutic antibody rituximab (black bars) was included at various concentrations. As a negative control, non-binding isotype control antibody was used in some conditions (grey bars) (top panel) When iPSCs were differentiated into iNK cells expressing the low affinity variant of CD16, ADCC was evident (increased dead tumor cells) only when rituximab was included. (bottom panel) When iPSCs were differentiated into iNK cells expressing the high affinity variant of CD16, greater ADCC was observed compared to the low affinity version of CD16.

FIG. 8 shows a gating strategy for ADCC assay. Lymphocytes were gated based on forward scatter area (FSC-A) and side scatter area (SSC-A), followed by gating on CellTrace Violet (CTV)+ target cells (Raji cells in FIG. 9 and RajiACD19 cells in FIG. 10), and finally gating on 7-AAD-positive cells to determine % of dead therapeutic iNK target cells. FSC-A=forward scatter area, SSC-A=side scatter area, CTV=CellTrace Violet, 7-AAD=7-amino-actinomycin D.

FIGS. 9A and 9B show rituximab-mediated ADCC using therapeutic iNK cells and Raji target cells. Rituximab at different concentrations (10, 1, 0.1, 0.01 and 0 g/mL rituximab; right bars with circles) was tested and compared to corresponding concentrations of a host-matched isotype control (left bars with squares). The percentage of 7-AAD+ cells on CTV-labeled targets were graphed by antibody concentration. The test effector cells included (i) therapeutic iNK cells expressing a low-affinity CD16 variant (iPSC16), (ii) therapeutic iNK cells expressing a high-affinity CD16 variant (iPSC17 or iPSC18), (iii) therapeutic iNK cells expressing both a low-affinity CD16 variant (iPSC16) and a CD19-specific CAR (a p1209 transgene encoding a CD19-specific CAR), and (iv) therapeutic iNK cells expressing both a high-affinity CD16 variant (iPSC17 or iPSC18) and a CD19-specific CAR (a p1209 transgene encoding a CD19-specific CAR). FIG. 9A shows results of an ADCC assay of control iNK effector cells that do not express a CD16 variant (left graphs) and iNK effector cells expressing a low affinity CD16 variant (iPSC16; right graphs). FIG. 9B shows results of an ADCC assay of iNK effector cells expressing a high affinity CD16 variant (iPSC17 or iPSC18; left and middle graphs). The results of a control ADCC assay with Raji target cells alone are shown in FIG. 9B (right bar graphs).

FIGS. 10A and 10B show rituximab-mediated ADCC using therapeutic iNK cells and RajiACD19 target cells. Rituximab at different concentrations (10, 1, 0.1, 0.01 and 0 g/mL rituximab; right bars with circles) was tested and compared to corresponding concentrations of a host-matched isotype control (left bars with squares). The percentage of 7-AAD+ cells on CTV-labeled targets were graphed by antibody concentration. The test effector cells included (i) therapeutic iNK cells expressing a low-affinity CD16 variant (iPSC16), (ii) therapeutic iNK cells expressing a high-affinity CD16 variant (iPSC17 or iPSC18), (iii) therapeutic iNK cells expressing both a low-affinity CD16 variant (iPSC16) and a CD19-specific CAR (a p1209 transgene encoding a CD19-specific CAR), and (iv) therapeutic iNK cells expressing both a high-affinity CD16 variant (iPSC17 or iPSC18) and a CD19-specific CAR (a p1209 transgene encoding a CD19-specific CAR). FIG. 10A shows results of an ADCC assay of control iNK effector cells that do not express a CD16 variant (left graphs) and iNK effector cells expressing a low affinity CD16 variant (iPSC16; right graphs). FIG. 10B shows results of an ADCC assay of iNK effector cells expressing a high affinity CD16 variant (iPSC17 or iPSC18; left and middle graphs). The results of a control ADCC assay with RajiACD19 target cells alone are shown in FIG. 10B (right graphs).

### DETAILED DESCRIPTION

### I. Introduction

Provided herein are induced pluripotent stem cell (iPSC)-derived cell therapies such as iPSC-derived natural killer (iNK) cells and iPSC-derived T (iT) cells for immuno-oncology. In some aspects, the genetically engineered iPSC-derived immune cells express CARs and other molecules that can mediate the persistence, functionality, and/or activation of these engineered immune cells. Also, provided herein are methods of generating and using such iPSC-derived immune cells.

Additionally described are genetically engineered iPSCs and cells derived therefrom that exogenously express recombinant CD16 and recombinant NKG2D. In some aspects, such cells also express a CAR. Also provided are related constructs (e.g., vectors), polynucleotides, and pharmaceutical compositions.

### II. Definitions

Unless defined otherwise, all terms of art, notations and other technical and scientific terms or terminology used herein have the same meaning as is commonly understood by one of skill in the art to which the claimed subject matter belongs. In some cases, terms with commonly understood meanings are defined herein for clarity and/or for ready reference, and the inclusion of such definitions herein should not necessarily be construed to represent a substantial difference over what is generally understood in the art. All patents and publications referred to herein are incorporated by reference in their entireties.

Unless defined otherwise, all technical and scientific terms, acronyms, and abbreviations used herein have the same meaning as commonly understood by one of ordinary skill in the art to which the invention pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A (alone)”, and “B (alone)”.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus.

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

Unless otherwise indicated, the term “at least” preceding a series of elements is to be understood to refer to every element in the series. Those skilled in the art will recognize or be able to ascertain using no more than routine experimentation, many equivalents to the specific embodiments of the application described herein. Such equivalents are intended to be encompassed by the application.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The terms “identical” or percent “identity,” in the context of two or more nucleic acids or polypeptide sequences (e.g., CAR polypeptides and the CAR polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide, protein, or cell) has been substantially separated, produced apart from, or purified away from other biological components of the organism in which the component naturally occurs, i.e., other chromosomal and extrachromosomal DNA and RNA, proteins, cells, and tissues. Nucleic acids, peptides, proteins, and cells that have been “isolated” thus include nucleic acids, peptides, proteins, and cells purified by standard purification methods and purification methods described herein. “Isolated” nucleic acids, peptides, proteins, and cells can be part of a composition and still be isolated if the composition is not part of the native environment of the nucleic acid, peptide, protein, or cell. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides,” “nucleic acids,” or “polynucleic acids,” refers to any polyribonucleotide or polydeoxyribonucleotide, which can be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” can further comprise replacement of the endogenous sequence or a nucleotide that is deleted with the one or more inserted nucleotides.

As used herein, the term “exogenous” is intended to mean that the referenced molecule or the referenced activity is introduced into, or non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell in its native form. Similarly, the term when used in reference to expression of an encoding nucleic acid refers to expression of an encoding nucleic acid natively contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof, a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “engineered immune cell” refers to an immune cell, also referred to as an immune effector cell, that has been genetically modified by the addition of exogenous genetic material in the form of DNA or RNA to the total genetic material of the cell.

As used herein, a “porcine tesehovirus-1 2A peptide” or “P2A peptide” or “P2A”, refers to a “self-cleaving peptide” of a picornavirus. The average length of P2A peptides is 18-22 amino acids. A P2A peptide was first identified in a foot-and-mouth disease virus (FMDV), a member of the picornavirus (Ryan et al., J Gen Virol, 1991, 72(Pt 11): 2727-2732). It was reported that ribosomes skip the synthesis of the glycyl-prolyl peptide bond at the C-terminus of a 2A peptide, leading to the cleavage between a 2A peptide and its immediate downstream peptide (see, e.g., Donnelly et al., J Gen Virol., 2001, 82: 1013-1025.

As used herein, the term “differentiation” is the process by which an unspecialized (“uncommitted”) or less specialized cell acquires the features of a specialized cell. Specialized cells include, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma or the embryo proper. For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the terms “reprogramming” or “dedifferentiation” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “induced pluripotent stem cells” or “iPSCs”, means that the stem cells are produced from differentiated adult, neonatal or fetal cells that have been induced or changed or reprogrammed into cells capable of differentiating into tissues of all three germ or dermal layers: mesoderm, endoderm, and ectoderm. The iPSCs produced do not refer to cells as they are found in nature.

The term “hematopoietic stem and progenitor cells,” “hematopoietic stem cells,” “hematopoietic progenitor cells,” or “hematopoietic precursor cells” or “HPCs” refers to cells which are committed to a hematopoietic lineage but are capable of further hematopoietic differentiation. Hematopoietic stem cells include, for example, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). As used herein, “CD34+ hematopoietic progenitor cell” refers to an HPC that expresses CD34 on its surface.

As used herein, the term “immune cell” or “immune effector cell” refers to a cell that is involved in an immune response. Immune response includes, for example, the promotion of an immune effector response. Examples of immune cells include T cells, B cells, natural killer (NK) cells, mast cells, and myeloid-derived phagocytes.

As used herein, the terms “T lymphocyte” and “T cell” are used interchangeably and refer to a type of white blood cell that completes maturation in the thymus and that has various roles in the immune system. A T cell can have the roles including, e.g., the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupTl, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Thl and Th2 cells), CD8+ T cells (e.g., cytotoxic T cells), peripheral blood mononuclear cells (PBMCs), peripheral blood leukocytes (PBLs), tumor infiltrating lymphocytes (TTLs), memory T cells, naive T cells, regulator T cells, gamma delta T cells (gd T cells or γδ T cells), and the like. Additional types of helper T cells include cells such as Th3 (Treg), Thl7, Th9, or Tfh cells. Additional types of memory T cells include cells such as central memory T cells (Tcm cells), effector memory T cells (Tern cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

“CD4+ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

As used herein, the term “CD8+ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refers to a subset of peripheral blood lymphocytes defined by the expression of CD56 and CD45 and the absence of the T cell receptor (TCR chains). The NK cell can also refer to a genetically engineered NK cell, such as an NK cell modified to express a chimeric antigen receptor (CAR). The NK cell can also be differentiated from a stem cell or progenitor cell.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK or “iNK” cells or derivative T or “iT” cells, as used throughout this application are cells differentiated from an iPSC, as compared to their primary counterpart obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSC using genomic editing.

As used herein, the term “chimeric antigen receptor” (CAR) refers to a recombinant polypeptide comprising at least an extracellular domain that binds specifically to an antigen or a target, a transmembrane domain and an intracellular signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen binding domain,” “extracellular domain,” or “extracellular ligand binding domain” refers to the part of a CAR that is located outside of the cell membrane and is capable of binding to an antigen, target or ligand.

As used herein, the term “hinge region” or “hinge domain” refers to the part of a CAR that connects two adjacent domains of the CAR protein, i.e., the extracellular domain and the transmembrane domain of the CAR protein.

As used herein, the term “transmembrane domain” refers to the portion of a CAR that extends across the cell membrane and anchors the CAR to cell membrane.

The term “hinge region” or “spacer region” as used herein generally means any oligo- or polypeptide that functions to link the extracellular domain to the transmembrane domain. A hinge region can be used to provide more flexibility and accessibility for the extracellular domain.

As used herein, the term “intracellular signaling domain,” “cytoplasmic signaling domain,” or “intracellular signaling domain” refers to the part of a CAR that is located inside of the cell membrane and is capable of transducing an effector signal.

As used herein, the term “stimulatory molecule” refers to a molecule expressed by an immune cell (e.g., NK cell or T cell) that provides the primary cytoplasmic signaling sequence(s) that regulate primary activation of receptors in a stimulatory way for at least some aspect of the immune cell signaling pathway. Stimulatory molecules comprise two distinct classes of cytoplasmic signaling sequence, those that initiate antigen-dependent primary activation (referred to as “primary signaling domains”), and those that act in an antigen-independent manner to provide a secondary of co-stimulatory signal (referred to as “co-stimulatory signaling domains”).

In certain embodiments, the extracellular domain comprises an antigen binding domain and/or an antigen binding fragment. The antigen binding fragment can, for example, be an antibody or antigen binding fragment thereof that specifically binds a tumor antigen. The antigen binding fragments of the application possess one or more desirable functional properties, including but not limited to high-affinity binding to a tumor antigen, high specificity to a tumor antigen, the ability to stimulate complement-dependent cytotoxicity (CDC), antibody-dependent phagocytosis (ADPC), and/or antibody-dependent cellular-mediated cytotoxicity (ADCC) against cells expressing a tumor antigen, and the ability to inhibit tumor growth in subjects in need thereof and in animal models when administered alone or in combination with other anti-cancer therapies.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the application can be of any of the five major classes or corresponding sub-classes. Preferably, the antibodies of the application are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the application can contain a kappa or lambda light chain constant domain. According to particular embodiments, the antibodies of the application include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to the specific tumor antigen is substantially free of antibodies that do not bind to the tumor antigen). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the application can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb), a scFv dimer (bivalent diabody), a multispecific antibody formed from a portion of an antibody comprising one or more CDRs, a camelized single domain antibody, a minibody, a nanobody, a domain antibody, a bivalent domain antibody, a light chain variable domain (VL), a variable domain (VHH) of a camelid antibody, or any other antibody fragment that binds to an antigen but does not comprise a complete antibody structure. An antigen-binding fragment is capable of binding to the same antigen to which the parent antibody or a parent antibody fragment binds.

As used herein, the term “single-chain antibody” refers to a conventional single-chain antibody in the field, which comprises a heavy chain variable region and a light chain variable region connected by a short peptide of about 15 to about 20 amino acids (e.g., a linker peptide).

As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, a bispecific antibody comprises a VHH having binding specificity for a first epitope, and a VHH having binding specificity for a second epitope.

As used herein, an antigen binding domain or antigen binding fragment that “specifically binds to a tumor antigen” refers to an antigen binding domain or antigen binding fragment that binds a tumor antigen, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” or “Kd” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antigen binding domain or antigen binding fragment can be determined by using surface plasmon resonance, such as by using a biosensor system, e.g., a Biacore® system, or by using bio-layer interferometry technology, such as an Octet RED96 system. The smaller the value of the KD of an antigen binding domain or antigen binding fragment, the higher affinity that the antigen binding domain or antigen binding fragment binds to a target antigen.

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted or disrupted at the insertion site during targeted editing, an endogenous gene comprising the affected sequence can be knocked-out or knocked-down due to the sequence deletion or disruption. Therefore, targeted editing can also be used to disrupt endogenous gene expression with precision. Similarly used herein is the term “targeted integration,” referring to a process involving insertion of one or more exogenous sequences at pre-selected sites in the genome, with or without deletion of an endogenous sequence at the insertion site.

As used herein, the terms “cancer”, “malignancy”, “neoplasm”, “tumor”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for treatment in the present application include precancerous (e.g., benign), malignant, pre-metastatic, metastatic, and non-metastatic cells. The teachings of the present disclosure may be relevant to any and all cancers. Non-limiting examples of one or more cancers include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

### III. NKG2D and CD16 Transgene

Described herein is a method for exogenously expressing or overexpressing CD16 and NKG2D proteins and transgenes in cells, as well as such cells and therapeutic uses thereof. The surface receptor CD16 (FcγRIIIA) affects human natural killer (NK) cells during maturation. NK cells bind the Fc portion of IgG via CD16, and execute antibody-dependent cellular cytotoxicity, which is critical for the effectiveness of several anti-tumor monoclonal antibody therapies. NKG2D is an stimulatory/activating receptor that is mostly expressed on cells of the cytotoxic arm of the immune system including NK cells and subsets of T cells. NKG2D is crucial in diverse aspects of innate and adaptive immune functions. In some embodiments, CD16 and NKG2D are expressed from in a single polynucleotide construct as it is advantageous to reduce the number of gene edits of a cell.

In some aspects, provided is an iPSC cell or derivative cell thereof containing an exogenous or isolated polynucleotide construct encoding a CD16 protein and an NKG2D protein. In some embodiments, described herein is an iPSC cell or derivative cell thereof expressing recombinant CD16 proteins and recombinant NKG2D proteins. In some embodiments, the recombinant proteins are encoded by an exogenous or isolated polynucleotide construct. In some embodiments, the polynucleotide construct encoding the CD16 protein and the NKG2D protein also includes a polynucleotide sequence encoding an autoprotease peptide or self-cleaving peptide. In some embodiments, an exogenous polynucleotide construct encoding the CD16 protein, the NKG2D protein and the self-cleaving peptide is introduced into the iPSC cell or derivative cell thereof. The exogenous or isolated polynucleotide construct can be introduced into a gene locus of the iPSC cell or derivative cell thereof.

In some embodiments, the iPSC cell or derivative cell thereof expressing recombinant CD16 proteins and recombinant NKG2D proteins also expresses chimeric antigen receptors (CARs). In some embodiments, the cell expressing recombinant CD16 proteins and recombinant NKG2D proteins also expresses either recombinant HLA-E, HLA-G, or both. In several embodiments, the iPSC cell or derivative cell thereof expressing recombinant CD16 proteins and recombinant NKG2D proteins also expresses CARs and either recombinant HLA-E, HLA-G, or both. In many embodiments, the cell expressing recombinant CD16 proteins, recombinant NKG2D proteins and CARs also expresses recombinant IL-15 proteins. In many embodiments, the cell expresses recombinant CD16 proteins, recombinant NKG2D proteins, CARs, recombinant IL-15 proteins, and either recombinant HLA-E, HLA-G, or both.

In many embodiments, the cell expressing recombinant CD16 proteins, recombinant NKG2D proteins and CARs also expresses recombinant fusion proteins containing IL-15 and IL-15Rα. In many embodiments, the cell expresses recombinant CD16 proteins, recombinant NKG2D proteins, CARs, recombinant fusion proteins containing IL-15 and IL-15Rα, and either recombinant HLA-E, HLA-G, or both. In some embodiments, the cell expressing recombinant CD16 proteins and recombinant NKG2D proteins also expresses recombinant IL-15 proteins. In some embodiments, the cell expressing recombinant CD16 proteins and recombinant NKG2D proteins also expresses recombinant fusion proteins containing IL-15 and IL-15Rα. In some embodiments, the cell expressing recombinant CD16 proteins, recombinant NKG2D proteins, and recombinant IL-15 proteins also expresses CARs. In some embodiments, the cell expressing recombinant CD16 proteins, recombinant NKG2D proteins, and recombinant fusion proteins containing IL-15 and IL-15Rα also expresses CARs.

In one aspect, provided is an exogenous or isolated polynucleotide construct encoding a CD16 protein and an NKG2D protein. In some embodiments of the exogenous polynucleotide construct, the polynucleotide sequence encoding a CD16 protein and the polynucleotide sequence encoding an NKG2D protein are operably linked by a polynucleotide sequence encoding an autoprotease peptide or self-cleaving peptide. In some embodiments, the polynucleotide construct includes from 5′ to 3′ end: a polynucleotide sequence encoding a CD16 protein, a polynucleotide sequence encoding an autoprotease peptide or self-cleaving peptide and a polynucleotide sequence encoding an NKG2D protein. In some embodiments, the polynucleotide construct includes from 5′ to 3′ end: a polynucleotide sequence encoding an NKG2D protein, a polynucleotide sequence encoding an autoprotease peptide or self-cleaving peptide and a polynucleotide sequence encoding a CD16 protein. In some embodiments, the exogenous polynucleotide construct comprises the nucleic acid sequence of SEQ ID NO:6. In some embodiments, the exogenous polynucleotide construct encodes for the amino acid sequence of SEQ ID NO:1.

In some embodiments, the CD16 protein (which is also referred to as “low affinity immunoglobulin gamma Fc region receptor III-A” or “Fc gamma receptor IIIa”) is a wildtype CD16 protein. In some embodiments, the human wildtype CD16 protein has the amino acid sequence set forth in NCBI Ref. Seq. No. NP_000560.7 or UniProt No. P08637. In some instance, the coding sequence of human wildtype CD16 is set forth in NCBI Ref. No. NM_000569.8.

In some embodiments, the CD16 protein is a CD16 variant protein. In some instances, the CD16 variant protein has an amino acid sequence having at least 90%, e.g., at least 90%, at least 91%, at least 92%, at least 93%, at least 94%, at least 95%, at least 96%, at least 97%, at least 98%, or at least 99% sequence identity to wildtype CD16 such as that of SEQ ID NO:5. In some instances, the CD16 variant is a high affinity CD16 variant. In other instances, the CD16 variant is a non-cleavable CD16 variant. In some instances, the CD16 variant is a high affinity and non-cleavable CD16 variant.

In some embodiments, the CD16 variant comprises one or more amino acid substitutions selected from the group consisting of F158V, F176V, S197P, D205A, S219A, T220A, and any combination thereof. In some embodiments, the CD16 variant has an F158V substitution and one or more substitutions selected from F176V, S197P, D205A, S219A, T220A, and any combination thereof. In one embodiment, the CD16 variant has an F176V substitution and one or more substitutions selected from F158V, S197P, D205A, S219A, T220A, and any combination thereof. In many embodiments, the CD16 variant has an S197P, substitution and one or more substitutions selected from F158V, F176V, D205A, S219A, T220A, and any combination thereof. In various embodiments, the CD16 variant has a D205A substitution and one or more substitutions selected from F158V, F176V, S197P, S219A, T220A, and any combination thereof. In some embodiments, the CD16 variant has a substitution and one or more substitutions selected from F158V, F176V, S197P, D205A, S219A, T220A, and any combination thereof. In some embodiments, the CD16 variant has an S219A substitution and one or more substitutions selected from F158V, F176V, S197P, D205A, T220A, and any combination thereof. In some embodiments, the CD16 variant has a T220A substitution and one or more substitutions selected from F158V, F176V, S197P, D205A, S219A, T220A, and any combination thereof. In some embodiments, the CD16 variant protein has a F176V substitution. In some embodiments, the variant CD16 protein has the sequence of SEQ ID NO:2. In some embodiments, the nucleic acid sequence encoding the variant CD16 protein has the sequence of SEQ ID NO:7. In some embodiments, the wildtype CD16 protein has the sequence of SEQ ID NO:5.

In some embodiments, the NKG2D protein (which is also referred to as NKG2-D type II integral membrane protein, CD314, killer cell lectin-like receptor subfamily K1 member 1 or KLRK1) is a wildtype NKG2D protein. In some embodiments, the human wildtype NKG2D protein has the amino acid sequence set forth in NCBI Ref. Seq. Nos. NP_001186734.1 or NP_031386.2 or UniProt No. P26718. In some instance, the coding sequence of human wildtype NKG2D is set forth in NCBI Ref. Nos. NM_001199805.1 or NM_007360.3. In some embodiment, the NKG2D protein is an NKG2D variant protein. In some instances, the NKG2D variant protein has an amino acid sequence having at least 90%, e.g., at least 90%, at least 91%, at least 92%, at least 93%, at least 94%, at least 95%, at least 96%, at least 97%, at least 98%, or at least 99% sequence identity to wildtype NKG2D such as that of SEQ ID NO:4. In some embodiment, the NKG2D protein has the amino acid sequence of SEQ ID NO:4. In some embodiment, the nucleic acid sequence encoding the NKG2D protein has sequence of SEQ ID NO:9.

### A. Autoprotease Peptides

As discussed above, provided herein are constructs containing autoprotease peptide sequences including 2A peptides that can induce ribosomal skipping during translation of an polypeptide. 2A peptides function to “cleave” an mRNA transcript by making the ribosome skip the synthesis of a peptide bond at the C-terminus, between the glycine (G) and proline (P) residues, thereby leading to separation between the end of the 2A sequence and the next peptide downstream. 2A peptides include, but are not limited to, a porcine tesehovirus-1 2A (P2A) peptide, a foot-and-mouth disease virus (FMDV) 2A (F2A) peptide, an Equine Rhinitis A Virus (ERAV) 2A (E2A) peptide, a Thosea asigna virus 2A (T2A) peptide, a cytoplasmic polyhedrosis virus 2A (BmCPV2A) peptide, and a Flacherie Virus 2A (BmIFV2A) peptide.

An exemplary P2A peptide can include an amino acid sequence having at least 90%, such as 90%, 91%, 92%, 93%, 94%, 95%, 96%, 97%, 98%, 99% or 100% sequence identity to SEQ ID NO:3. In some embodiment, the P2A peptide has the amino acid sequence of SEQ ID NO:3.

### IV. Chimeric Antigen Receptors (CARs)

In some embodiments, an iPSC cell or derivative cell thereof contains an exogenous polynucleotide encoding a chimeric antigen receptor (CAR), such as a CAR targeting a tumor antigen. In some instances, the recombinant CAR polypeptide includes at least an extracellular domain that binds specifically to an antigen (or more than one antigen), a transmembrane domain and an intracellular signaling domain. In some instances, the recombinant CAR polypeptide includes a signal peptide, an extracellular domain that binds specifically to an antigen (or more than one antigen), a transmembrane domain, an intracellular signaling domain and one or more co-stimulatory domains. In other instances, the recombinant CAR polypeptide includes a signal peptide, an extracellular domain that binds specifically to an antigen (or more than one antigen), a hinge region, a transmembrane domain, an intracellular signaling domain and one or more co-stimulatory domains.

### A. Extracellular Domains

In certain embodiments, an extracellular domain of a CAR includes an antibody, an antibody fragment, an antigen-binding domain and/or an antigen-binding fragment. The antigen binding fragment can, for example, be an antibody or antigen-binding fragment thereof that specifically binds a tumor antigen. In some embodiments, the antigen-binding domains or fragments possess one or more desirable functional properties including, but not limited to, high-affinity binding to a tumor antigen, high specificity to a tumor antigen, the ability to stimulate complement-dependent cytotoxicity (CDC), antibody-dependent phagocytosis (ADPC), and/or antibody-dependent cellular-mediated cytotoxicity (ADCC) against cells expressing a tumor antigen, and the ability to inhibit tumor growth in subjects in need thereof and in animal models when administered alone or in combination with other anti-cancer therapies.

In some embodiments, antibodies or antibody fragments suitable for use in the CAR include, but are not limited to, monoclonal antibodies, bispecific antibodies, multispecific antibodies, chimeric antibodies, polypeptide-Fc fusions, single-chain Fvs (scFv), single chain antibodies, Fab fragments, F(ab′) fragments, disulfide-linked Fvs (sdFv), masked antibodies (e.g., Probodies®), Small Modular ImmunoPharmaceuticals (“SMIPs™”), intrabodies, minibodies, single domain antibody variable domains, nanobodies, VHHs, diabodies, tandem diabodies (TandAb®), anti-idiotypic (anti-Id) antibodies (including, e.g., anti-Id antibodies to antigen-specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the antigen-binding fragment is an Fab fragment, an Fab′ fragment, an F(ab′)2 fragment, an scFv fragment, an Fv fragment, a dsFv diabody, a VHH, a VNAR, a single-domain antibody (sdAb) or nanobody, a dAb fragment, a Fd′ fragment, a Fd fragment, a heavy chain variable region, an isolated complementarity determining region (CDR), a diabody, a triabody, or a decabody. In some embodiments, the antigen-binding fragment is an scFv fragment. In some embodiments, the antigen-binding fragment is a VHH.

In some embodiments, the extracellular domain of the CAR is a single-domain antibody or nanobody. In some embodiments, the extracellular domain is a VHH. In some embodiments, the extracellular domain is an scFv.

Optionally, alternative scaffolds to immunoglobulin domains that exhibit similar functional characteristics, such as high-affinity and specific binding of target biomolecules, may also be used in the CARs described. Such scaffolds have been shown to yield molecules with improved characteristics, such as greater stability or reduced immunogenicity. Non-limiting examples of alternative scaffolds include engineered, tenascin-derived, tenascin type III domain (e.g., Centyrin™); engineered, gamma-B crystallin-derived scaffold or engineered, ubiquitin-derived scaffold (e.g., Affilins); engineered, fibronectin-derived, 10th fibronectin type III (10Fn3) domain (e.g., monobodies, AdNectins™, or AdNexins™); engineered, ankyrin repeat motif containing polypeptide (e.g., DARPins™); engineered, low-density-lipoprotein-receptor-derived, A domain (LDLR-A) (e.g., Avimers™); lipocalin (e.g., anticalins); engineered, protease inhibitor-derived, Kunitz domain (e.g., EETI-II/AGRP, BPTI/LACI-D1/ITI-D2); engineered, Protein-A-derived, Z domain (Affibodies™); Sac7d-derived polypeptides (e.g., Nanoffitins® or affitins); engineered, Fyn-derived, SH2 domain (e.g., Fynomers®); CTLD3 (e.g., Tetranectin); thioredoxin (e.g., peptide aptamer); KALBITOR®; the β-sandwich (e.g., iMab); miniproteins; C-type lectin-like domain scaffolds; engineered antibody mimics; and any genetically manipulated counterparts of the foregoing that retains its binding functionality (Wörn A, Pluckthun A, J Mol Biol 305: 989-1010 (2001); Xu L et al., Chem Biol 9: 933-42 (2002); Wikman M et al., Protein Eng Des Sel 17: 455-62 (2004); Binz H et al., Nat Biotechnol 23: 1257-68 (2005); Hey T et al., Trends Biotechnol 23:514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Skerra, Curr Opin in Biotech., 2007 18: 295-304; Byla P et al., J Biol Chem 285: 12096 (2010); Zoller F et al., Molecules 16: 2467-85 (2011), each of which is incorporated by reference in its entirety). In some embodiments, the alternative scaffold is Affilin or Centyrin.

i. Antigen-Binding Domains

In some embodiments, an antigen-binding domain of a CAR binds to a target antigen. The antigen-binding domain may bind to more than one antigen or more than one epitope in an antigen. For example, the antigen-binding domain may bind to 2, 3, 4, 5, 6, 7, 8 or more antigens. As another example, the antigen-binding domain may bind 2, 3, 4, 5, 6, 7, 8 or more epitopes in the same antigen.

The choice of antigen-binding domain may depend upon the type and number of antigens that define the surface of a target cell. For example, the antigen-binding domain may be chosen to recognize an antigen that acts as a cell surface marker on target cells associated with a particular disease state. In some embodiments, CAR can be genetically modified to target a tumor antigen of interest by way of engineering a desired antigen-binding domain that specifically binds to an antigen (e.g., on a tumor cell). Non-limiting examples of cell surface markers that may act as targets for the antigen-binding domain in the CAR include those associated with tumor cells or autoimmune diseases.

In some embodiments, the antigen-binding domain binds to at least one tumor antigen or autoimmune antigen.

In some embodiments, the antigen-binding domain binds to at least one tumor antigen. In some embodiments, the antigen-binding domain binds to two or more tumor antigens. In some embodiments, the two or more tumor antigens are associated with the same tumor. In some embodiments, the two or more tumor antigens are associated with different tumors.

In some embodiments, the antigen-binding domain binds to at least one autoimmune antigen. In some embodiments, the antigen-binding domain binds to two or more autoimmune antigens. In some embodiments, the two or more autoimmune antigens are associated with the same autoimmune disease. In some embodiments, the two or more autoimmune antigens are associated with different autoimmune diseases.

In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. Non-limiting examples of tumor antigen associated with glioblastoma include HER2, EGFRvIII, EGFR, CD133, PDGFRA, FGFR1, FGFR3, MET, CD70, ROBO1 and IL13Rα2. Non-limiting examples of tumor antigens associated with ovarian cancer include FOLR1, FSHR, MUC16, MUC1, Mesothelin, CA125, EpCAM, EGFR, PDGFRα, Nectin-4 and B7H4. Non-limiting examples of the tumor antigens associated with cervical cancer or head and neck cancer include GD2, MUC1, Mesothelin, HER2, and EGFR. Non-limiting examples of tumor antigen associated with liver cancer include Claudin 18.2, GPC-3, EpCAM, cMET, and AFP. Non-limiting examples of tumor antigens associated with hematological malignancies include CD19, CD22, CD79, BCMA, GPRC5D, SLAM F7, CD33, CLL1, CD123, and CD70. Non-limiting examples of tumor antigens associated with bladder cancer include Nectin-4 and SLITRK6. Non-limiting examples of tumor antigens associated with renal cancer include CD70 and FOLR1.

Additional examples of antigens that may be targeted by the antigen-binding domain include, but are not limited to, alpha-fetoprotein, A3, antigen specific for A33 antibody, Ba 733, BrE3-antigen, carbonic anhydrase EX, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

In one embodiment, the antigen targeted by the antigen-binding domain is CD19. In one embodiment, the antigen-binding domain comprises an anti-CD19 scFv. In one embodiment, the anti-CD19 scFv comprises a heavy chain variable region (VH) comprising the amino acid sequence set forth in SEQ ID NO:2, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity with SEQ ID NO:2 as set forth in PCT/US2021/072646. In one embodiment, the anti-CD19 scFv comprises a light chain variable region (VL) comprising the amino acid sequence set forth in SEQ ID NO: 4, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity with SEQ ID NO:4 as set forth in PCT/US2021/072646. In one embodiment, the anti-CD19 scFv comprises the amino acid sequence set forth in SEQ ID NO: 7, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity with SEQ ID NO: 7 as set forth in PCT/US2021/072646.

In some embodiments, the antigen is associated with an autoimmune disease or disorder. Such antigens may be derived from cell receptors and cells which produce “self”-directed antibodies. In some embodiments, the antigen is associated with an autoimmune disease or disorder such as Rheumatoid arthritis (RA), multiple sclerosis (MS), Sjögren's syndrome, Systemic lupus erythematosus, sarcoidosis, type 1 diabetes mellitus, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Myasthenia gravis, Hashimoto's thyroiditis, Graves' disease, chronic inflammatory demyelinating polyneuropathy, Guillain-Barre syndrome, Crohn's disease or ulcerative colitis.

In some embodiments, autoimmune antigens that may be targeted by the CAR include, but are not limited to, platelet antigens, myelin protein antigen, Sm antigens in snRNPs, islet cell antigen, rheumatoid factor, and anticitrullinated protein. citrullinated proteins and peptides such as CCP-1, CCP-2 (cyclical citrullinated peptides), fibrinogen, fibrin, vimentin, filaggrin, collagen I and II peptides, alpha-enolase, translation initiation factor 4G1, perinuclear factor, keratin, Sa (cytoskeletal protein vimentin), components of articular cartilage such as collagen II, IX, and XI, circulating serum proteins such as RFs (IgG, IgM), fibrinogen, plasminogen, ferritin, nuclear components such as RA33/hnRNP A2, Sm, eukaryotic translation elongation factor 1 alpha 1, stress proteins such as HSP-65, -70, -90, BiP, inflammatory/immune factors such as B7-H1, IL-1 alpha, and IL-8, enzymes such as calpastatin, alpha-enolase, aldolase-A, dipeptidyl peptidase, osteopontin, glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase, cathepsin G, myeloperoxidase, proteinase 3, platelet antigens, myelin protein antigen, islet cell antigen, rheumatoid factor, histones, ribosomal P proteins, cardiolipin, vimentin, nucleic acids such as dsDNA, ssDNA, and RNA, ribonuclear particles and proteins such as Sm antigens (including but not limited to SmD's and SmB′/B), U1RNP, A2/B1 hnRNP, Ro (SSA), and La (SSB) antigens.

Non-limiting exemplary antigen targets are provided in Tables 1-3. Table 1 provides antigen binding domains that bind to exemplary antigen targets. The antigen-binding domain may comprise a VH sequence, a VL sequence, and/or CDRs thereof, such as those described in the cited publications, the contents of each publication are incorporated herein by reference in their entirety for all purposes.

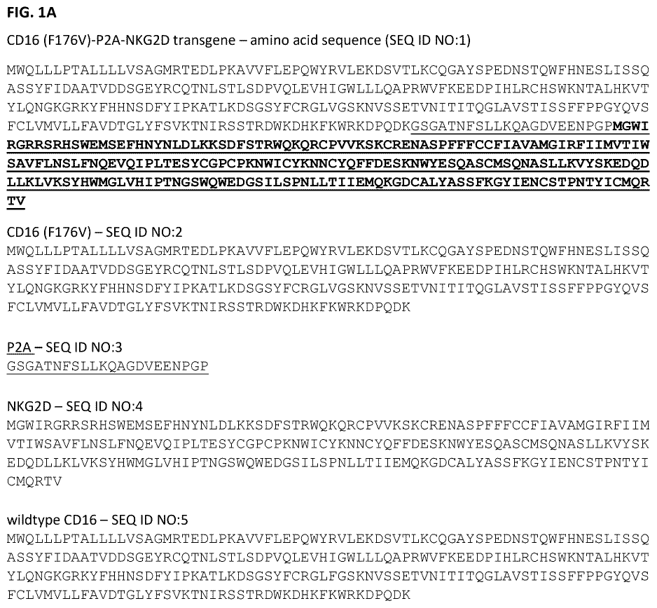

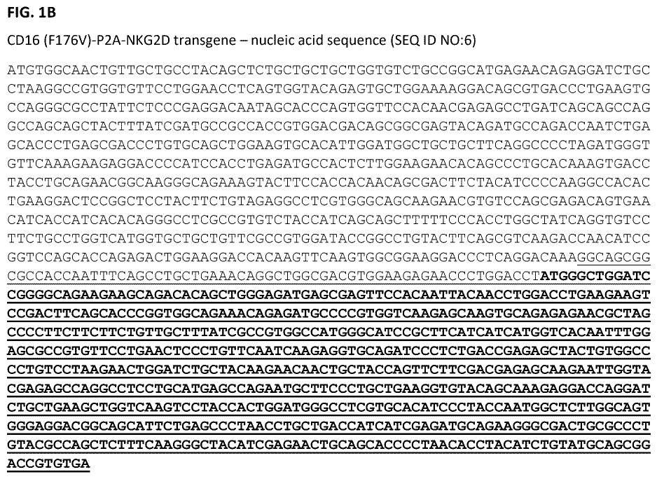

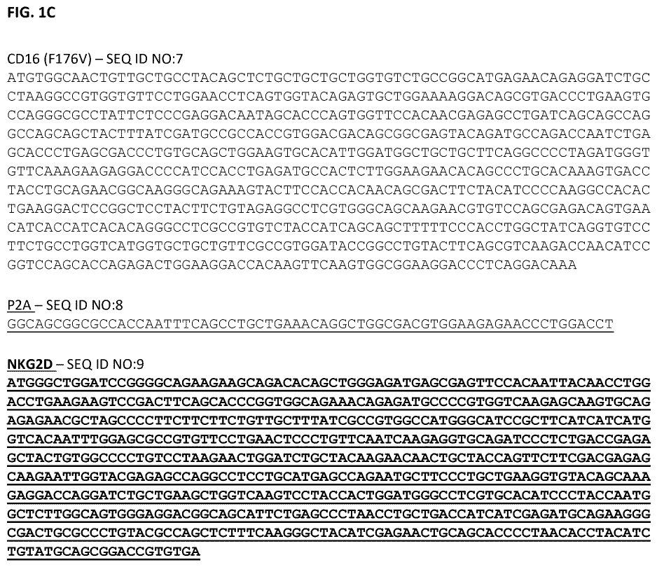

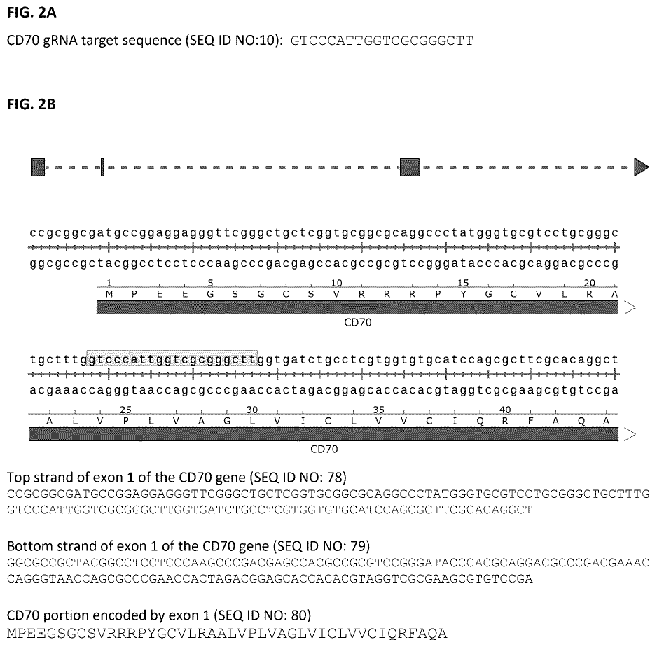

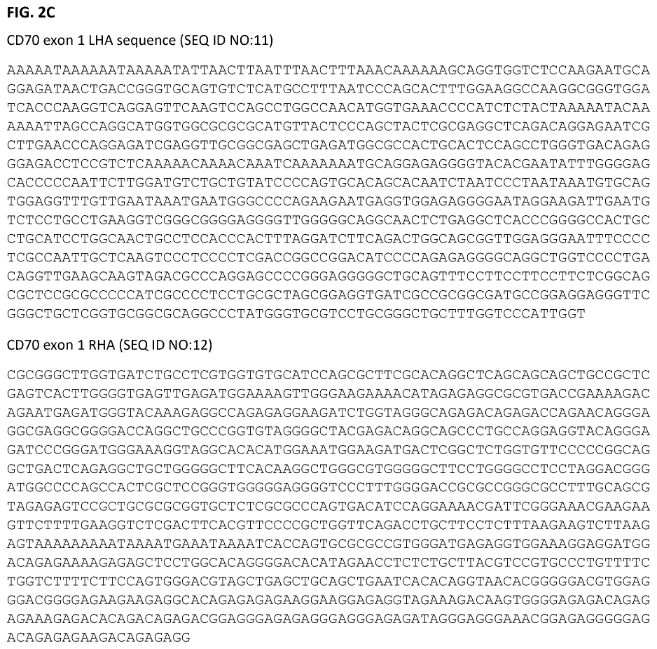

TABLE 1

| Antigen target | Type | Examples of Source | Type | Examples of Source |
|---|---|---|---|---|
| 5T4 | VH | Identifier 2, 4 in | VL | Identifier 1, 3 in |
| | | WO2016022939 | | WO2016022939 |
| AGR2 | VH | Identifier 10, 18 in | VL | Identifier 11, 19 in |
| | | WO2016040321 | | WO2016040321 |
| ALK | VH | Identifier 1, 11, 13, 15, 3, 5, | VL | Identifier 10, 12, 14, 16, 2, |
| | | 7, 9 in US20160280798Al; | | 4, 6, 8 in |
| | | Identifier 9, 1, 3, 5, 11, 13, | | US20160280798Al; |
| | | 15, 7, 9 in WO2015069922 | | Identifier 10, 12, 14, 16, 8 |
| | | | | in WO2015069922; |
| | | | | Identifier 2, 4, 6 in |
| | | | | WO2015069922 |
| AMC | VH | Identifier 17, 18, 19, 20, 21, | VL | Identifier 27, 28, 29, 31, |
| | | 22, 23, 24, 25, 26 in | | 32, 33, 34, 35, 36 in |
| | | WO2016161390 | | WO2016161390 |
| ANG2 | VH | Identifier 1, 3 in | VL | Identifier 2 , 4 in |
| | | WO2015091655 | | WO2015091655 |
| APCDD1 | VH | Identifier 10, 102, 106, 110, | VL | Identifier 136, 100, 104, |
| | | 114, 118, 122, 126, 130, | | 108, 112, 116, 12, 120, |
| | | 134, 14, 6, 98 in | | 124, 128, 132, 16, 8 in |
| | | WO2012019061 | | WO2012019061 |
| APRIL | VH | Identifier 12, 14, 16. 18. 3. | VL | Identifier 20, 22, 24, 26, |
| | | 32, 34, 36, 38, 40, 42, 44, | | 28, 30, 4, 50 in |
| | | 46, 48, 52 in | | US2016026467 |
| | | US20160264674 | | |
| AXL | VH | Identifier 21, 3, 45 in | VL | Identifier 22, 4 in |
| | | WO2016097370 | | WO2016097370 |
| B2MG | VH | Identifier 28 in | VL | Identifier 29 in |
| | | WO2016126213A1 | | WO2016126213A1 |
| B7H1 | VH | Identifier 12, 32, 42, 52, 72, | VL | Identifier 17, 37, 47, 57, 7, |
| | | 2, 62 in US20130034559 | | 77, 27, 67 in |
| | | | | US20130034559 |
| B7H3 | VH | Identifier 10, 11, 12, 13, 14, | VL | Identifier 1, 2, 3, 4, 5, 6, 7, |
| | | 15, 16, 9 in WO2016033225 | | 8 in WO2016033225 |
| B7H3 (CD276) | VH | Identifier 17, 26, 7 in | VL | Identifier 18, 27, 8 in |
| | | WO2016044383 | | WO2016044383 |
| B7H4 | VH | Identifier 100, 101, 102, | VL | Identifier 104, 11, 126, |
| | | 103, 107, 108, 109, 110, | | 134, 138, 19, 27, 3, 35, 55, |
| | | 111, 112, 113, 114, 12, 127, | | 93, 95, 97, 98, 145, 146, |
| | | 130, 131, 132, 133, 137, 2, | | 147, 148 in |
| | | 20, 28, 36, 37, 38, 4, 56, 99, | | US20160159910; Identifier |
| | | 144 in US20160159910; | | 29, 31, 33 in |
| | | Identifier 13, 15, 17 in | | WO2016160620 |
| | | WO2016160620 | | |
| BAT1 | VH | Identifier 5, 6, 7, 8, 9 in | VL | Identifier 1, 2, 3, 4 in |
| | | WO2013014668 | | WO2013014668 |
| BCMA | VH | Identifier 26 in | VL | Identifier 25 in |
| | | WO2016168773 A3; | | WO2016168773 A3; |
| | | Identifier 142, 148, 154, | | Identifier 42 in |
| | | 160, 166, 172, 178, 184, | | WO2016097231; Identifier |
| | | 190, 196, 202, 208, 214, | | 143, 149, 155, 161, 167, |
| | | 220, 226, 232, 238, 244, | | 173, 179, 185, 191, 197, |
| | | 250, 256, 262, 268, 274, | | 203, 209, 215, 221, 227, |
| | | 280, 286, 292, 298, 304, | | 233, 239, 245, 251, 257, |
| | | 310, 316, 322, 328, 334, | | 263, 269, 275, 281, 287, |
| | | 340, 346, 352 in | | 293, 299, 305, 311, 317, |
| | | WO2016168595A1; | | 323, 329, 335, 341, 347, |
| | | Identifier 8 in | | 353 in WO2016168595 |
| | | WO2016094304A3; | | A1; Identifier 192, 193, |
| | | Identifier 171, 172, 173, | | 194, 195, 196, 197, 198, |
| | | 174, 175, 176, 177, 178, | | 199, 200, 201, 204, 205, |
| | | 179, 180, 181, 182, 183, | | 207, 208, 211, 259, 260, |
| | | 184, 185, 186, 187, 190, | | 84, 85, 86, 87, 88, 89, 90, |
| | | 255, 257, 258, 69, 70, 71, | | 91, 92, 93, 94, 95, 96, 97, |
| | | 72, 73, 74, 75, 76, 77, 78, | | 98 in WO2016014565; |
| | | 79, 80, 81 WO2016014565; | | Identifier 53 in |
| | | Identifier 38 in | | WO2016187349A1; |
| | | EP3057994A1; Identifier 55 | | Identifier 7 in |
| | | in WO2016187349A1; | | WO2016094304 A3; |
| | | Identifier 1, 13, 17, 21, 25, | | Identifier 10, 14, 18, 2, 22, |
| | | 29, 33, 37, 41, 45, 49, 5, 53, | | 26, 30, 34, 38, 42, 46, 50, |
| | | 57, 61, 65, 9 in | | 54, 58, 6, 62, 66 in |
| | | WO2016090320; Identifier | | WO2016090320; Identifier |
| | | 101, 743, 174, 758, 95, 759, | | 100, 102, 175, 96, 98 in |
| | | 97, 760, 99 in | | WO2016120216; Identifier |
| | | WO2016120216; Identifier | | 12, 14, 16, 18 in |
| | | 11, 741, 17 in | | WO2015158671A1; |
| | | WO2015158671A1; | | Identifier 7, 8, 9 in |
| | | Identifier 10, 11, 12, 13, 14 | | WO2016014789; Identifier |
| | | in WO2016014789; | | 14 in WO2016168766A1 |
| | | Identifier 15 in | | |
| | | WO2016168766A1 | | |
| BMPR1A | VH | Identifier 12 in | — | — |
| | | WO2011116212 | | |
| CA19.9 | VH | Identifier 117 in | VL | Identifier 118 in |
| | | US20160333114A1 | | US20160333114A1 |
| Campath 1 | VH | Identifier 34 in | VL | Identifier 31, 33 in |
| | | US20160333114A1 | | US20160333114A1 |
| CD105 | VH | Identifier 13, 14, 16 in | VL | Identifier 1, 17, 20, 22, 23 |
| | | WO2014039682 | | in WO2014039682 |
| CD123 | VH | Identifier 11, 13, 14, 21 in | VL | Identifier 9, 11, 18, 19, 20, |
| | | WO2015140268A1; | | 21, 22, 23 in |
| | | Identifier 113, 115, 57, 59, | | WO2016120220; Identifier |
| | | 63 in WO2016120216; | | 12, 16, 18, 19, 22 in |
| | | Identifier 12, 123, 24, 25, | | WO2015140268A1; |
| | | 26, 27, 28, 29, 30, 9 in | | Identifier 275, 276, 277, |
| | | WO2016120220; Identifier | | 278, 307, 308, 309, 310 in |
| | | 216, 217, 218, 219, 274 in | | WO2016028896; Identifier |
| | | WO2016028896 | | 5 in US20160333108A1; |
| | | | | Identifier 114, 116, 58, 60, |
| | | | | 64 in WO2016120216 |
| CD148 | VH | Identifier 10, 14, 18, 2, 22, | VL | Identifier 12, 16, 20, 24, |
| | | 26, 30, 6 in WO2005118643 | | 28, 32, 4, 8 in |
| | | | | WO2005118643 |
| CD16 | VH | Identifier 25 in | VL | Identifier 26 in |
| | | WO2015158868 | | WO2015158868 |
| CD19 | VH | Identifier 53, 55 in | VL | Identifier 27, 31 in |
| | | WO2016120216 | | WO2016168773 A3; |
| | | | | Identifier 49 in |
| | | | | WO2016187349A1; |
| | | | | Identifier 11 in |
| | | | | WO2016134284; Identifier |
| | | | | 194 in US20140134142A1; |
| | | | | Identifier 54, 56 in |
| | | | | WO2016120216; Identifier |
| | | | | 13, 14, 15, 16, 17, 186, |
| | | | | 187, 188, 189, 192, 196, |
| | | | | 197, 198, 199, 200, 201, |
| | | | | 202, 203, 204, 205, 64, 66, |
| | | | | 67, 68, 69, 70, 71, 91 |
| | | | | US20160152723; Identifier |
| | | | | 22 in US20160039942; |
| | | | | 3361 Identifier 63 in |
| | | | | WO2016097231; Identifier |
| | | | | 3 in US20160145337A1; |
| | | | | Identifier 112 in |
| | | | | US20160333114A1; |
| | | | | Identifier 114 in |
| | | | | US20160333114A1; |
| | | | | Identifier 13, 6 |
| | | | | US20160319020 |
| CD19H 803 | VH | Identifier 28, 29, 32, 33, 34, | — | — |
| | | 35 in WO2016168773 A3; | | |
| | | Identifier 51 in | | |
| | | WO2016187349A1; | | |
| | | Identifier 20 in | | |
| | | US20160039942; Identifier | | |
| | | 1 in WO2014184143; | | |
| | | Identifier 5 in | | |
| | | US20160145337A1; | | |
| | | Identifier 166, 167, 168, | | |
| | | 172, 176, 177, 181, 183, | | |
| | | 184, 185, 62 in | | |
| | | US20160152723; Identifier | | |
| | | 15 US20160319020; | | |
| | | Identifier 17, 33, 34, 35 in | | |
| | | EP3057994A1; Identifier 62 | | |
| | | in WO2016097231; | | |
| | | Identifier 12 in | | |
| | | WO2016134284; Identifier | | |
| | | 111, 113 in | | |
| | | US20160333114A1 | | |
| CD2 | VH | Identifier 103, 117, 119 in | VL | Identifier 102, 116 in |
| | | WO2016122701 | | WO2016122701 |
| CD20 | VH | Identifier 45 in | VL | Identifier 46 in |
| | | WO2016097231; Identifier | | WO2016097231; Identifier |
| | | 11, 13, 14, 15, 16, 17, 18, | | 10, 12, 8 in |
| | | 19, 20, 21, 22, 23, 24, 25, | | WO2017004091; Identifier |
| | | 26, 27, 28, 29, 30, 31, 32, | | 51 in US20160333114A1 |
| | | 33, 7, 9 in WO2017004091; | | |
| | | Identifier 26 in | | |
| | | US20170000900; Identifier | | |
| | | 54 in US20160333114A1; | | |
| | | Identifier 25 in | | |
| | | US20170000900; Identifier | | |
| | | 24 in US20170000900; | | |
| | | Identifier 23 in | | |
| | | US20170000900 | | |
| CD22 | VH | Identifier 3 in | VL | Identifier 17, 8, 14, 15 in |
| | | WO2013059593; Identifier | | US20150239974; Identifier |
| | | 10, 11, 12, 7, 9, 8 in | | 7 in US20150299317; |
| | | US20150299317; Identifier | | Identifier 681 in |
| | | 201 in WO2016164731; | | WO2016164731; Identifier |
| | | CD22 VH 869 Identifier | | 682 in WO2016164731; |
| | | 671 in WO2016164731; | | Identifier 683, 2020 in |
| | | Identifier 672 in | | WO2016164731; Identifier |
| | | WO2016164731; Identifier | | 684 in WO2016164731; |
| | | 673 in WO2016164731; | | Identifier 685 in |
| | | Identifier 676 in | | WO2016164731; Identifier |
| | | WO2016164731; Identifier | | 686 in WO2016164731; |
| | | 678 in WO2016164731; | | Identifier 687 in |
| | | Identifier 679 in | | WO2016164731; Identifier |
| | | WO2016164731; Identifier | | 688 in WO2016164731; |
| | | 680 in WO2016164731; | | Identifier 690 in |
| | | Identifier 700 in | | WO2016164731; Identifier |
| | | WO2016164731; Identifier | | 740 in WO2016164731; |
| | | 701 in WO2016164731; | | Identifier 741 in |
| | | Identifier 702 in | | WO2016164731; Identifier |
| | | WO2016164731; Identifier | | 742 in WO2016164731; |
| | | 703 in WO2016164731; | | Identifier 743 in |
| | | Identifier 704 in | | WO2016164731; Identifier |
| | | WO2016164731; Identifier | | 744 in WO2016164731; |
| | | 705 in WO2016164731; | | Identifier 745 in |
| | | Identifier 706 in | | WO2016164731; Identifier |
| | | WO2016164731; Identifier | | 746 in WO2016164731; |
| | | 707 in WO2016164731; | | Identifier 747 in |
| | | Identifier 708 in | | WO2016164731; Identifier |
| | | WO2016164731, Identifier | | 748 in WO2016164731; |
| | | 709 in WO2016164731; | | Identifier 749 in |
| | | Identifier 711 in | | WO2016164731; Identifier |
| | | WO2016164731; Identifier | | 750 in WO2016164731; |
| | | 712 in WO2016164731; | | Identifier 752 in |
| | | Identifier 713 in | | WO2016164731; Identifier |
| | | WO2016164731; Identifier | | 753 in WO2016164731; |
| | | 714 in WO2016164731; | | Identifier 754 in |
| | | Identifier 715 in | | WO2016164731; Identifier |
| | | WO2016164731; Identifier | | 755 in WO2016164731; |
| | | 716 in WO2016164731; | | Identifier 756 in |
| | | Identifier 717 in | | WO2016164731; Identifier |
| | | WO2016164731; Identifier | | 757 in WO2016164731; |
| | | 718 in WO2016164731; | | Identifier 758 in |
| | | Identifier 719 in | | WO2016164731; Identifier |
| | | WO2016164731; Identifier | | 759 in WO2016164731; |
| | | 720 in WO2016164731; | | Identifier 760 in |
| | | Identifier 721 in | | WO2016164731; Identifier |
| | | WO2016164731; Identifier | | 761 in WO2016164731; |
| | | 722 in WO2016164731; | | Identifier 762 in |
| | | Identifier 723 in | | WO2016164731; Identifier |
| | | WO2016164731; Identifier | | 763 in WO2016164731; |
| | | 724 in WO2016164731; | | Identifier 764 in |
| | | Identifier 725 in | | WO2016164731; Identifier |
| | | WO2016164731; Identifier | | 765 in WO2016164731; |
| | | 726 in WO2016164731; | | Identifier 766 in |
| | | Identifier 727 in | | WO2016164731; Identifier |
| | | WO2016164731; Identifier | | 767 in WO2016164731; |
| | | 728 in WO2016164731; | | Identifier 768 in |
| | | Identifier 729 in | | WO2016164731; Identifier |
| | | WO2016164731; Identifier | | 769 in WO2016164731; |
| | | 730 in WO2016164731; | | Identifier 770 in |
| | | Identifier 731 in | | WO2016164731; Identifier |
| | | WO2016164731; Identifier | | 771 in WO2016164731; |
| | | 732 in WO2016164731; | | Identifier 772 in |
| | | Identifier 733 in | | WO2016164731; Identifier |
| | | WO2016164731; Identifier | | 773 in WO2016164731; |
| | | 734 in WO2016164731; | | Identifier 774 in |
| | | Identifier 735 in | | WO2016164731; Identifier |
| | | WO2016164731; Identifier | | 775 in WO2016164731; |
| | | 736 in WO2016164731; | | Identifier 776 in |
| | | Identifier 737 in | | WO2016164731; Identifier |
| | | WO2016164731; Identifier | | 777 in WO2016164731; |
| | | 738 in WO2016164731 | | Identifier 124 in |
| | | | | WO2016122701 |
| CD276 | VH | Identifier 17, 26, 7 in | VL | Identifier 18, 27 in |
| | | US20160053017 | | US20160053017 |
| CD28 | VH | Identifier 19 in | VL | Identifier 20 in |
| | | WO2015158868 | | WO2015158868 |
| CD3 | VH | Identifier 108, 112, 115 in | VL | Identifier 104 in |
| | | WO2016122701; Identifier | | WO2016122701; Identifier |
| | | 29 in WO2014144722 A2; | | 13 in WO2016126213A1 |
| | | Identifier 12 in | | |
| | | WO2016126213A1 | | |
| CD30 | VH | Identifier 14, 16 in | VL | Identifier 13, 15 in |
| | | WO2016134284 | | WO2016134284 |
| CD324 | VH | Identifier 21, 23, 25, 27, 29, | VL | Identifier 20, 22, 24, 26, |
| | | 31, 33, 35, 37, 39, 41, 43, | | 28, 30, 32, 34, 36, 38, 40, |
| | | 45, 47, 49, 51, 53, 55, 57, | | 42, 44, 46, 48, 50, 52, 54, |
| | | 59, 61, 63, 65, 67, 69, 71 in | | 56, 58, 60, 62, 64, 66, 68, |
| | | U.S. Pat. No. 9,534,058 | | 70 in U.S. Pat. No. 9,534,058 |
| CD32B | VH | Identifier 127 in | VL | Identifier 126 in |
| | | WO2016122701 | | WO2016122701 |
| CD33 | VH | Identifier 65, 67, 69, 71, 77, | VL | Identifier 12, 14, 16, 18 in |
| | | 79, 81, 83, 84 in | | WO2015150526A2; |
| | | WO2016120216; Identifier | | Identifier 66, 68, 70, 72, |
| | | 11, 13, 15; 17 in | | 78, 80, 82 in |
| | | WO2015150526A2; | | WO2016120216; Identifier |
| | | Identifier 57, 58, 59, 60, 61, | | 66, 67, 68, 69, 70, 71, 72, |
| | | 62, 63, 64, 65 in | | 73, 74 in WO2016014576 |
| | | WO2016014576 | | |
| CD37 | VH | Identifier 11, 12, 18 in | VL | Identifier 14, 15 in |
| | | US20170000900 | | US20170000900 |
| CD38 | VH | Identifier 2 in | VL | Identifier 1, 11 in |
| | | WO2009080830; Identifier | | WO2009080830 |
| | | 10 in WO2015121454 | | |
| CD3s | VH | Identifier 7 in | VL | Identifier 8 in |
| | | WO2014144722A2 | | WO2014144722A2 |
| CD40 | VH | Identifier 1 in | VL | Identifier 2 in |
| | | WO2016069919; Identifier | | WO2016069919; Identifier |
| | | 5, 7, 8 in WO2015091655 | | 6 in WO2015091655 |
| CD45 | VH | Identifier 24 in | VL | Identifier 25 in |
| | | WO2016126213A1 | | WO2016126213A1 |
| CD46 | VH | Identifier 39, 47, 59, 15, 19, | VL | Identifier 41, 61, 21, 25, |
| | | 23, 27, 31, 35, 43, 51, 55, | | 29, 33, 37, 45, 49, 53, 57, |
| | | 63, 67, 71, 75, 79, 83, 69, | | 65, 69, 73, 77, 81, 85, 17, |
| | | 71, 83 in WO2012031273; | | 73, 77 in WO2012031273; |
| | | Identifier 1, 10, 11, 12, 13, | | Identifier 23, 24, 25, 26, |
| | | 14, 15, 16, 17, 3, 5, 6, 7, 9, | | 27, 28, 29, 30, 31, 32, 33, |
| | | 18, 19, 20, 21 in | | 34, 35, 36, 37, 38, 39, 40, |
| | | WO2016040683 | | 41, 42 in WO2016040683 |
| CD4BS | VH | Identifier 15, 3 in | VL | Identifier 14, 2 in |
| | | US20160194375A1 | | US20160194375A1 |
| CD4i | VH | Identifier 5 in | VL | Identifier 4 in |
| | | US20160194375A1 | | US20160194375A1 |
| CD52 | VH | Identifier 103, 136, 137 in | VL | Identifier 102, 138 in |
| | | WO2010132659 | | WO2010132659 |
| CD64 | VH | Identifier 129 in | VL | Identifier 128 in |
| | | WO2016122701 | | WO2016122701 |
| CD7 | VH | Identifier 16, 20 in | VL | Identifier 17, 21 in |
| | | WO2016126213A1 | | WO2016126213A1 |
| CD70 | VH | Identifier 81, 85, 89 in | VL | Identifier 83, 87, 91 in |
| | | WO2015121454 | | WO2015121454; Identifier |
| | | | | 83 in WO2015121454; |
| | | | | Identifier 87 in |
| | | | | WO2015121454; Identifier |
| | | | | 91 in WO2015121454 |
| CD71 | VH | Identifier 1, 3, 325, 4, 5, 699 | VL | Identifier 2,327, 329, 331, |
| | | in US20160355599 | | 333, 335, 337, 6, 650, 652, |
| | | | | 654, 656, 658, 660, 670, |
| | | | | 671, 672, 673, 7, 701, 702, |
| | | | | 703, 704, 705, 706, 707, |
| | | | | 708, 709, 710, 711, 712, |
| | | | | 721, 722, 723, 724, 725, |
| | | | | 726, 727, 728, 729, 730, |
| | | | | 731, 732, 733, 734, 735, |
| | | | | 736, 737, 738, 739, 740, |
| | | | | 741, 742, 743, 744, 745, |
| | | | | 746, 747, 748, 749, 750, |
| | | | | 751, 752, 753, 754, 755, |
| | | | | 756, 757, 758, 759, 760, |
| | | | | 761, 762, 763, 764, 765, |
| | | | | 766, 767, 768, 769, 770, |
| | | | | 771, 772, 773, 774, 775, |
| | | | | 776, 777, 778, 779, 780, |
| | | | | 781, 782, 783, 784 785, |
| | | | | 786, 787, 788, 8, 810, 811, |
| | | | | 812, 813, 814, 815, 816, |
| | | | | 817, 818, 819, 820, 821, |
| | | | | 822, 823, 824, 825, 826, |
| | | | | 827, 828, 829, 830, 831, |
| | | | | 832, 833, 834, 835, 836, |
| | | | | 810, 842, 843, 844, 845, |
| | | | | 846, 847, 848, 849, 850, |
| | | | | 851, 852, 853, 854, 855, |
| | | | | 856, 857, 858, 859, 860, |
| | | | | 861, 862, 863, 864, 865, |
| | | | | 866, 867, 868, 869, 870, |
| | | | | 871, 872, 873, 874, 875, |
| | | | | 876, 877, 879, 879, 880, |
| | | | | 881, 882, 883, 884, 885, |
| | | | | 886, 887, 888, 889, 890, |
| | | | | 891, 892, 893, 894, 895, |
| | | | | 896, 897, 898, 899, 900, |
| | | | | 901, 902, 903, 904, 905, |
| | | | | 906, 907, 908 in |
| | | | | US20160355599 |
| CD73 | VH | Identifier 100, 103, 107, | VL | Identifier 12. 20, 44, 72, |
| | | 109, 112, 114, 116, 119, | | 76, 8, 84, 92 in |
| | | 121, 16, 32, 4, 52, 60, 68, | | US20160145350; Identifier |
| | | 80, 88 in US20160145350; | | 22, 29, 37, 4 in |
| | | Identifier 135, 40, 21, 3, 28, | | WO2016055609A1; |
| | | 36 in WO2016055609A1 | | Identifier 101, 102, 104, |
| | | | | 106, 110, 117, 118, 120, |
| | | | | 122 in US20160145350 |
| CD74 | VH | Identifier 6 in | VL | Identifier 25, 29, 31, 35 in |
| | | US20100284906A1; | | US20130171064; Identifier |
| | | Identifier 10, 11, 9 in | | 12, 13, 14, 11, 4 in |
| | | US20040115193A1; | | US20040115193A1 |
| | | Identifier 23, 27, 30, 33 in | | |
| | | US20130171064 | | |
| CD76b | VH | Identifier 15, 17, 19, 23, 27, | VL | Identifier 16, 18, 22, 38, |
| | | 29, 37, 57, 59, 61 in | | 58, 60, 62 in |
| | | US20160159906 | | US20160159906 |
| CD79 | VH | Identifier 131 in | VL | Identifier 130 in |
| | | WO2016122701 | | WO2016122701 |
| CDIM | VH | Identifier 1, 10, 11, 12, 13, | VL | Identifier 28, 29, 30, 31, |
| | | 14, 15, 16, 17, 18, 19, 2, 20, | | 32, 33, 34, 35, 36, 37, 38, |
| | | 21, 22, 3, 4, 5, 6, 7, 8, 9 in | | 39, 40, 41, 42, 43, 44, 45, |
| | | WO2013120012 | | 46, 47, 48, 49 in |
| | | | | WO2013120012 |
| CEA | VH | Identifier 8 in U.S. Pat. No. 8,287,865 | VL | Identifier 10, 38, 39, 7, 9 in |
| | | | | U.S. Pat. No. 8,287,865 |
| Claudin | VH | Identifier 101, 103, 105, | VL | Identifier 114, 116, 118, |
| | | 107, 109, 111, 113, 115, | | 120, 22, 25, 29, 33, 37, 41, |
| | | 117, 119, 121, 122, 123, | | 45, 49, 53, 57, 61, 65, 69, |
| | | 124, 125, 126, 127, 128, | | 73, 77 in |
| | | 129, 130, 131, 132, 133, | | WO2016073649A1 |
| | | 134, 135, 136, 137, 138, | | |
| | | 139, 140, 141, 142, 143, | | |
| | | 144, 145, 146, 147, 148, | | |
| | | 149, 150, 23, 27, 31, 35, 39, | | |
| | | 43, 47, 51, 55, 59, 63, 67, | | |
| | | 71, 75, 79, 81, 83, 85, 87, | | |
| | | 89, 91, 93, 95, 97, 99 in | | |
| | | WO2016073649A1 | | |
| CLDN18.2 | VH | Identifier 12, 2 in | VL | Identifier 13, 3 in |
| | | US20160347815A1 | | US20160347815A1 |
| CLL1 | VH | Identifier 13, 14, 15, 17, 19, | VL | Identifier 16, 18, 20, 22, |
| | | 21, 23, 25, 27, 29, 31, 33, 35 | | 24, 26, 28, 30, 32, 24, 36 in |
| | | in WO2016120219; | | WO2016120219; Identifier |
| | | Identifier 195, 65, 66, 67, | | 196, 78, 79, 80, 81, 82, 83, |
| | | 68, 69, 70, 71, 72, 73, 74, | | 84, 85, 86, 87, 88, 89, 90 in |
| | | 75, 76, 77 in | | WO2016014535; Identifier |
| | | WO2016014535; Identifier | | 30, 32, 35, 37, 39, 41 in |
| | | 31, 33, 34, 36, 38, 40, 42, 46 | | US20160075787; Identifier |
| | | in US20160075787; | | 152, 104, 106, 108, 110, |
| | | Identifier 150, 103, 105, | | 112, 114, 116, 118 in |
| | | 107, 109, 111, 113, 115, 117 | | WO2016179319A1 |
| | | in WO2016179319A1 | | |
| CLL3 | VH | Identifier 101, 103, 105, | VL | Identifier 100, 102, 104, |
| | | 107, 109, 111, 113, 115, | | 106, 108, 110, 112, 114, |
| | | 117, 119, 121, 123, 125, | | 116, 118, 120, 122, 124, |
| | | 127, 129, 131, 133, 135, | | 126, 128, 130, 132, 134, |
| | | 137, 139, 141, 145, 147, | | 136, 138, 140, 144, 146, |
| | | 149, 151, 153, 155, 157, | | 148, 150, 152, 154, 156, |
| | | 159, 161, 163, 165, 167, | | 158, 160, 162, 164, 166, |
| | | 169, 171, 173, 175, 177, | | 170, 172, 174, 176, 178, |
| | | 179, 181, 183, 185, 187, | | 180, 182, 184, 186, 190, |
| | | 191, 193, 195, 197, 199, | | 192, 194, 196, 198, 20, |
| | | 201, 203, 205, 207, 209, 21, | | 200, 202, 204, 206, 208, |
| | | 211, 213, 23, 25, 27, 29, 31, | | 210, 212, 22, 24, 26, 28, |
| | | 33, 35, 37, 39, 41, 43, 45, | | 30, 32, 34, 36, 38, 40, 42, |
| | | 47, 49, 51, 53, 55, 57, 59, | | 44, 46, 48, 50, 54, 56, 58, |
| | | 61, 63, 65, 67, 69, 71, 73, | | 60, 62, 64, 66, 68, 70, 72, |
| | | 75, 77, 79, 81, 83, 85, 87, | | 74, 76, 78, 80, 82, 84, 86, |
| | | 89, 91, 93, 95, 97, 99 in | | 88, 90, 92, 94, 96, 98 in |
| | | US20170000901 | | US20170000901 |
| collagen | VH | Identifier 21, 4, 15, 17, 18, | VL | Identifier 11, 12, 14, 23, |
| | | 19, 20, 5, 6, 7, 1, 2, 3 in | | 25, 26, 27, 8, 9 in |
| | | WO2007024921 | | WO2007024921 |
| CS1 | VH | Identifier 22 in | VL | Identifier 104, 106, 108 in |
| | | WO2016168773 A3; 103, | | WO2016120216; Identifier |
| | | 105, 107, 109 in | | 14, 16, 18, 20, 22 in |
| | | WO2016120216; Identifier | | WO2015166056A1; |
| | | 13, 15, 17, 19 in | | Identifier 39, 41, 43, 45, 47 |
| | | WO2015166056A1; | | in WO2015121454; |
| | | Identifier 38, 40, 42, 44, 46 | | Identifier 110, 112 in |
| | | in WO2015121454; | | WO2016120216 |
| | | Identifier 26 in | | |
| | | US20160075784A1 | | |
| CSF | VH | Identifier 10, 102, 14, 18, 2, | VL | Identifier 12, 32, 44, 48, 60 |
| | | 22, 26, 30, 34, 38, 46, 50, | | in US20050059113A1 |
| | | 54, 58, 6, 62, 66, 70, 74, 78, | | |
| | | 82, 86, 90, 94, 98 in | | |
| | | US20050059113A1 | | |
| CSPG4 | VH | Identifier 10, 16, 18, 4, 6, 8 | VL | Identifier 7 in |
| | | in WO2016077638; | | WO2016164429; Identifier |
| | | Identifier 8 in | | 12, 14 in WO2016077638 |
| | | WO2016164429 | | |
| CTLA4 | VH | Identifier 3, 31, 32, 33, 34, | VL | Identifier 36, 37, 38, 39, |
| | | 35, 41, 42, 43, 44, 45, 7 in | | 40, 46, 47, 48, 49, 50, 8, 4 |
| | | US20140105914; Identifier | | in US20140105914; |
| | | 4 in U.S. Pat. No. 8,697,845; Identifier | | Identifier 2 in U.S. Pat. No. 8,697,845; |
| | | 19 in US20150283234; | | Identifier 20 in |
| | | Identifier 17 in | | US20150283234; Identifier |
| | | WO2014066532 | | 18 in WO2014066532 |
| CXCR4 | VH | Identifier 72, 73, 74, 75, 84 | VL | Identifier 76, 77, 78, 79, |
| | | in US20110020218 | | 80, 81, 82, 87, 88, 90, 91, |
| | | | | 92, 93 in US20110020218 |
| Daclizumab | VH | Identifier 44, 46 in | VL | Identifier 43, 45 in |
| | | US20160333114A1 | | US20160333114A1 |
| DR5 | VH | Identifier 18, 82, 90, 98, 8 in | VL | Identifier 13, 23, 25, 27, 3, |
| | | WO2016122701 | | 78, 86, 94, 29 in |
| | | | | WO2016122701; Identifier |
| | | | | 62 in WO2016122701; |
| | | | | Identifier 54 in |
| | | | | WO2016122701; Identifier |
| | | | | 70 in WO2016122701 |
| E7MC | VH | Identifier 15, 16, 17, 18, 19, | VL | Identifier 238, 239, 240, |
| | | 20, 21, 22, 23, 233, 234, | | 241, 242, 243, 36, 37, 38, |
| | | 235, 236, 237, 24, 25, 26, | | 39, 41, 42, 43, 44, 45, 46, |
| | | 27, 28, 29, 30, 31, 32, 33, | | 47, 48, 49, 50, 51, 52, 53, |
| | | 34, 35 in | | 54, 55, 56 in |
| | | WO2016182957A1 | | WO2016182957A1 |
| EFNA | VH | Identifier 149, 153, 157, 161 | VL | Identifier 151, 155, 159, |
| | | in US20150125472 | | 163 in WO2012118547; |
| | | | | Identifier 27, 53 in |
| | | | | US20150125472 |
| EFNA4 | VH | Identifier 13, 39 in | — | — |
| | | US20150125472 | | |
| EGFR | VH | Identifier 14, 50, 9 in | VL | Identifier 15 in |
| | | WO2015143382; Identifier | | WO2015143382; Identifier |
| | | 12, 14, 15, 21 in | | 14 in WO2014143765; |
| | | US20100008978A1; | | Identifier 4051, 4052, |
| | | Identifier 2123 in | | 4053, 4054, 4055, 4056, |
| | | WO2018231759 | | 4057, 4058, 4059, 4060, |
| | | | | 4061, 4062, 4063, 4064, |
| | | | | 4065, 4066, 4067, 4068, |
| | | | | 4069, 4070, 4071, 4072, |
| | | | | 4073, 4074, 4075, 4076, |
| | | | | 4077, 4078, 4079, 4080, |
| | | | | 4081, 4082, 4083, 4084, |
| | | | | 4085, 4086, 4087, 4088, |
| | | | | 4089. 4090, 4091, 4092, |
| | | | | 4093, 4094, 4095, 4096, |
| | | | | 4097, 4098, 4099, 4100, |
| | | | | 4101,4102, 4103, 4104, |
| | | | | 4105, 4106, 4107, 4108, |
| | | | | 4109, 4110, 4111, 4112, |
| | | | | 4113, 4114, 4115, 4116, |
| | | | | 4117, 4118, 4119, 4120, |
| | | | | 4121, 4122, 4123, 4124, |
| | | | | 4125, 4126, 4127, 4128, |
| | | | | 4129, 4130, 4131, 4132, |
| | | | | 4133, 4134, 4135, 4136, |
| | | | | 4137, 4138, 4139, 4140, |
| | | | | 4141, 4142, 4143, 4144, |
| | | | | 4145, 4146, 4147, 4148, |
| | | | | 4149, 4150, 4151, 4152, |
| | | | | 4153, 4154, 4155, 4156, |
| | | | | 4157, 4158, 4159, 4160, |
| | | | | 4161, 4162, 4163, 4164, |
| | | | | 4165, 4166, 4167, 4168, |
| | | | | 4169, 4170, 4171, 4172, |
| | | | | 4173, 4174, 4175, 4176, |
| | | | | 4177, 4178, 4179, 4180, |
| | | | | 4181, 4182, 4183, 4184, |
| | | | | 4185, 4186, 4187, 4188, |
| | | | | 4189, 4190, 4191, 4192, |
| | | | | 4193, 4194, 4195, 4196, |
| | | | | 4197, 4198, 4199, 4200, |
| | | | | 4201, 4202, 4203, 4204, |
| | | | | 4205, , 4206, 4207, 4208, |
| | | | | 4209, 4210, 4211, 4212, |
| | | | | 4213, 4214, 4215, 4216, |
| | | | | 4217, 4218, 4219, 4220, |
| | | | | 4221, 4222, 4223, 4224, |
| | | | | 4225, 4226, 4227, 4228, |
| | | | | 4229, 4230, 4231, 4232, |
| | | | | 4233, 4234, 4235, 4236, |
| | | | | 4237, 4238, 4239, 4240, |
| | | | | 4241, 4242, 4243, 4244, |
| | | | | 4245, 4246, 4247, 4248, |
| | | | | 4249, 4250, 4251, 4252, |
| | | | | 4253, 4254, 4255, 4256, |
| | | | | 4257, 4258, 4259, 4260, |
| | | | | 4261, 4262, 4263, 4264, |
| | | | | 4265, 4266, 4267, 4268, |
| | | | | 4269, 4270, 4271, 4272, |
| | | | | 4273, 4274, 4275, 4276, |
| | | | | 4277, 4278, 4279, 42870, |
| | | | | 4281, 4282, 42834284, |
| | | | | 4285, 4286, 4287, 4288, |
| | | | | 4289, 4290, 4291, 4292, |
| | | | | 4293, 4294, 4295, 4296, |
| | | | | 4297, 4298, 4299, 4300, |
| | | | | 4301, 4302, 4303, 4304, |
| | | | | 4305, 4306, 4307, 4308, |
| | | | | 4309, 4310, 4311, 4312, |
| | | | | 4313, 4314, 4315, 4316, |
| | | | | 4317, 4318, 4319, 4320, |
| | | | | 4321, 4322, 4323, 4324, |
| | | | | 4325, 4326, 4327, 4328, |
| | | | | 4329, 4330, 4331, 4332, |
| | | | | 4333, 4334, 4335, 4336, |
| | | | | 4337, 4338, 4339, 4340, |
| | | | | 4341, 4342, 4343, 4344, |
| | | | | 4345, 4346, 4347, 4348, |
| | | | | 4349, 4350, 4351, 4352, |
| | | | | 4353, 4354, 4355, 4356, |
| | | | | 4357, 4358, 4359, 4360, |
| | | | | 4361, 4362, 4363, 4364, |
| | | | | 4365, 4366, 4367, 4368, |
| | | | | 4369, 4370, 4371, 4372, |
| | | | | 4373, 4374, 4375, 4376, |
| | | | | 4377, 4378, 4379, 4380, |
| | | | | 4381, 4382, 4383, 4384, |
| | | | | 4385, 4386, 4387, 4388, |
| | | | | 4389, 4390, 4391, 4392, |
| | | | | 4393, 4394, 4395, 4396, |
| | | | | 4397, 4398, 4399, 4400, |
| | | | | 4401, 4402, 4403, 4404, |
| | | | | 4405, 4406, 4407, 4408, |
| | | | | 4409, 4410, 4411, 4412, |
| | | | | 4413, 4414, 4415, 4416, |
| | | | | 4417, 4418, 4419, 4420, |
| | | | | 4421, 4422, 4423, 4424, |
| | | | | 4425, 4426, 4427, 4428, |
| | | | | 4429, 4430, 4431, 4432, |
| | | | | 4433, 4434, 4435, 4436, |
| | | | | 4437, 4438, 4439, 4440, |
| | | | | 4441, 4442, 4443, 4444, |
| | | | | 4445, 4446, 4447, 4448, |
| | | | | 4449, 4450, 4451, 4452, |
| | | | | 4453, 4454, 4455, 4456, |
| | | | | 4457, 4458, 4459, 4460, |
| | | | | 4461, 4462, 4463, 4464, |
| | | | | 4465, 4466, 4467, 4468, |
| | | | | 4469, 4470, 4471, 4472, |
| | | | | 4473, 4474, 4475, 4476, |
| | | | | 4477, 4478, 4479, 4480, |
| | | | | 4481, 4482, 4483, 4484, |
| | | | | 4485, 4486, 4487, 4488, |
| | | | | 4489, 4490, 4491, 4492, |
| | | | | 4493, 4494, 4495, 4496, |
| | | | | 4497, 4498, 4499, 4500, |
| | | | | 4501, 4502, 4503, 4504, |
| | | | | 4505, 4506, 4507, 4508, |
| | | | | 4509, 4510, 4511, 4512, |
| | | | | 4513, 4514, 4515, 4516, |
| | | | | 4517, 4518, 4519, 4520, |
| | | | | 4521, 4522, 4523, 4524, |
| | | | | 4525, 4526, 4527, 4528, |
| | | | | 4529, 4530, 4531, 4532, |
| | | | | 4533, 4534, 4535, 4536, |
| | | | | 4537, 4538, 4539, 4540, |
| | | | | 4541, 4542, 4543, 4544, |
| | | | | 4545, 4546, 4547, 4548, |
| | | | | 4549, 4550, 4551, 4552, |
| | | | | 4553, 4554, 4555, 4556, |
| | | | | 4557, 4558, 4559, 4560, |
| | | | | 4561, 4562, 4563, 4564, |
| | | | | 4565, 4566, 4567, 4568, |
| | | | | 4569, 4570, 4571, 4572, |
| | | | | 4573, 4574, 4575, 4576, |
| | | | | 4577, 4578, 4579, 4580, |
| | | | | 4581, 4582, 4583, 4584, |
| | | | | 4585, 4586, 4587, 4588, |
| | | | | 4589, 4590, 4591, 4592, |
| | | | | 4593, 4594, 4595, 4596, |
| | | | | 4597, 4598, 4599, 4600, |
| | | | | 4601, 4602, 4603, 4604, |
| | | | | 4605, 4606, 4607, 4608, |
| | | | | 4609, 4610, 4611, 4612, |
| | | | | 4613, 4614, 4615, 4616, |
| | | | | 4617, 4618, 4619, 4620, |
| | | | | 4621, 4622, 4623, 4624, |
| | | | | 4625, 4626, 4627, 4629, |
| | | | | 4629, 4630, 4631, 4632, |
| | | | | 4633, 4634, 4635, 4636, |
| | | | | 4637, 4638, 4639 in |
| | | | | WO2018231759 |
| EGFR | VH | Identifier 2124 in | VL | 4640, 4641, 4642, 4643, |
| (EGFRvIII) | | WO2018231759; Identifier | | 4644, 4645, 4646, 4647, |
| | | 13 in WO2016016341; | | 4648, 4649, 4650, 4651, |
| | | Identifier 24 in | | 4652, 4653, 4654, 4655, |
| | | WO2016168773 A3; | | 4656, 4657, 4658 in |
| | | Identifier 34 in | | WO2018231759; Identifier |
| | | US20160304615; Identifier | | 14 in WO2016016341; |
| | | 2 in US20160200819A1; | | Identifier 23 in |
| | | Identifier 91, 93 in | | WO2016168773 A3; |
| | | WO2016120216 | | Identifier 42 in |
| | | | | US20160304615; Identifier |
| | | | | 1 in US20160200819A1; |
| | | | | Identifier 92, 94 in |
| | | | | WO2016120216 |
| Endoglin | VH | Identifier 41, 42, 43, 71, 73, | VL | Identifier 103, 88, 89, 90, |
| | | 75, 88, 89, 90, 91, 92 in | | 91, 92, 93, 94, 95, 96, 97, |
| | | US20160009811 | | 102, 100 in |
| | | | | WO2011041441; Identifier |
| | | | | 100, 102, 103, 3, 4, 5, 70, |
| | | | | 72, 74, 93, 94, 95, 96, 97 in |
| | | | | US20160009811 |
| EphA2 receptor | VH | Identifier 20, 22, 24, 32, 34, | VL | Identifier 26, 28, 30, 47, |
| | | 36, 37, 38, 40, 42, 43, 45, | | 48, 49, 50, 52, 78, 80 in |
| | | 74, 76 in US20150274824 | | US20150274824 |
| ERBB2 | VH | Identifier 2, 4 in | VL | Identifier 1 in |
| | | US20110129464; Identifier | | US20110129464; Identifier |
| | | 10, 2, 26, 30, 38, 4, 40, 42, | | 12, 16, 20, 24, 32, 36, 44, |
| | | 52, 54, 56, 57, 58, 6 in | | 50, 51, 53, 8 in |
| | | US20130089544; Identifier | | US20130089544; Identifier |
| | | 8 in US20130266564; | | 7 in US20130266564; |
| | | Identifier 1 in | | Identifier 3 in |
| | | US20150104443 | | US20110129464 |
| Factor D | VH | Identifier 17, 20, 27, 29, 30, | VL | Identifier 16, 18, 19, 26, 3 |
| | | 31, 32, 33, 4 in | | in US20160017052 |
| | | US20160017052 | | |
| Factor XII | VH | Identifier 15 in | VL | Identifier 17 in |
| | | WO2014089493 | | WO2014089493 |
| FAP | VH | Identifier 1, 5 in | VL | Identifier 2, 6 in |
| | | WO2015118030; Identifier | | WO2015118030; Identifier |
| | | 170, 172 in | | 171, 173 in |
| | | WO2016120216; Identifier | | WO2016120216; Identifier |
| | | 8 in US20160326265 A1 | | 9 in US20160326265A1 |
| FcRL5 | VH | Identifier 12, 16, 20, 24, 28, | VL | Identifier 11, 15, 19, 23, |
| (FcReceptorLike | | 32, 36, 4, 40, 44, 48, 8, 915, | | 27, 3, 31, 35, 39, 43, 47, 7, |
| 5) | | 919 in WO2016090337 | | 917, 921 WO2016090337 |
| FGFR3 | VH | Identifier 132, 134, 136 in | VL | Identifier 133, 135, 137, |
| | | U.S. Pat. No. 9,499,623 | | 139 in U.S. Pat. No. 9,499,623 |
| Frizzled | VH | Identifier 10 in | VL | Identifier 12, 14 in |
| Receptor | | WO2010037041 | | WO2010037041 |
| GAH | VH | Identifier 7 in | VL | Identifier 8 in |
| | | US20060057147A1 | | US20060057147A1 |
| GCC1 | VH | Identifier 1 in | VL | Identifier 4, 2 in |
| | | US20160030595A1 | | US20160030595A1 |
| GD2 | VH | Identifier 17 in | VL | Identifier 10, 2, 5, 7, 9 in |
| | | WO2016134284; Identifier | | US20130216528; Identifier |
| | | 1 in US20130216528; | | 11, 12 in WO2015132604; |
| | | Identifier 10, 9 in | | Identifier 18 in |
| | | WO2015132604; Identifier | | WO2016134284 |
| | | 3, 4, 6, 8 in | | |
| | | US20130216528 | | |
| GD3 | VH | Identifier 11, 13, 15, 17 in | VL | Identifier 12, 14, 16, 18 in |
| | | WO2016185035A1 | | WO2016185035A1 |
| Glyco epitope | VH | Identifier 7 in | VL | Identifier 10 in |
| and ErbBB I | | WO2012007167A1 | | WO2012007167A1 |
| Specific | | | | |
| GM2 | VH | Identifier 20, 22, 23, 26, 27, | VL | Identifier 21, 24, 25, 31, |
| | | 28, 29, 30 in | | 32, 33, 34, 35 in |
| | | US20090028877 | | US20090028877 |
| GPC3 | VH | Identifier 10, 14, 2, 3, 4, 5, | VL | Identifier 10, 14, 18, 22, |
| | | 6, 7, 8, 9 in | | 24, 26 in U.S. Pat. No. 9,409,994B2; |
| | | US20160208015 A1; | | Identifier 16, 31 in |
| | | Identifier 22 in | | US20160208015A1; |
| | | WO2016049459; Identifier | | Identifier 23 in |
| | | 12, 16, 20, 37, 8 in | | WO2016049459 |
| | | U.S. Pat. No. 9,409,994B2 | | |
| GPDL1 | VH | Identifier 20 in | — | — |
| | | US20160108123 | | |
| GPRC5D | VH | Identifier 13, 17, 21, 25, 29, | VL | Identifier 10, 14, 18, 2, 22, |
| | | 314, 326, 33, 338, 350, 362, | | 26, 30, 303, 315, 327, 339, |
| | | 37, 374, 386, 41, 45, 49, 5, | | 34, 351, 363, 375, 38, 387, |
| | | 53, 57, 61, 65, 69, 73, 77, | | 42, 46, 50, 54, 58, 6, 62, |
| | | 81, 85, 89, 93, 1, 9 in | | 66, 70, 74, 78, 82, 86, 94 in |
| | | WO2016090312 | | WO2016090312 |
| Her2 | VH | Identifier 141, 262, 264, | VL | Identifier 140 in |
| | | 266, 268, 270 in | | WO2016054555A2; |
| | | WO2016168773A3; | | Identifier 261, 263, 265, |
| | | Identifier 11 in U.S. Pat. No. 9,518,118; | | 267, 269 in |
| | | Identifier 62 in | | WO2016168773A3; |
| | | US20160333114A1; | | Identifier 10, 18, 23 in |
| | | Identifier 19, 24 in | | U.S. Pat. No. 9,518,118; Identifier 3 in |
| | | U.S. Pat. No. 9,518,118 | | WO2016168769A1; |
| | | | | Identifier 59, 61 in |
| | | | | US20160333114A1 |
| Her1/her3 | VH | Identifier 8 of | VL | Identifier 4 of |
| | | WO2016073629 | | WO2016073629 |
| HLAG | VH | Identifier 10, 8 in | VL | Identifier 18, 20 in |
| | | WO2016160622A2 | | WO2016160622A2 |
| HSP70 | VH | Identifier 11, 12 in | VL | Identifier 16, 17 in |
| | | WO2016120217 | | WO2016120217 |
| Human collagen | VH | Identifier 31 in | VL | Identifier 32 in |
| VII | | WO2016112870 | | WO2016112870 |
| Human CD79b | VH | Identifier 27 in | VL | Identifier 28, 30 in |
| | | WO2016112870 | | WO2016112870 |
| | | humanCD79b VH 2304 | | |
| | | Identifier 29 in | | |
| | | WO2016112870 | | |
| Human ERBB3 | VH | Identifier 19, 29, 38, 45, 55, | VL | Identifier 10, 20, 30, 39, |
| | | 61, 9 in WO2013052745 | | 46, 56, 62 in |
| | | | | WO2013052745 |
| ICOS | VH | Identifier 15, 16, 19, 23, 7 in | VL | Identifier 17, 18, 20, 24, 8 |
| | | US20160215059 | | in US20160215059 |
| IGFI | VH | Identifier 1, 3, 7 in | VL | Identifier 2, 4, 6, 8 in |
| | | WO2007118214; Identifier | | WO2007118214 |
| | | 7 in WO2015073575 | | |
| IGFR1 | VH | Identifier 7 in | VL | Identifier 8 in |
| | | WO2015073575 A2 | | WO2015073575 A2 |
| IL13 | VH | Identifier 302 in | VL | Identifier 303 in |
| | | US20160168242 | | US20160168242 |
| IL13Ra2 | VH | Identifier 7, 8 in | — | — |
| | | WO2016123143 | | |
| IL21 | VH | Identifier 2, 3 in | — | — |
| | | US20160145332 | | |
| IL33 | VH | Identifier 134, 136, 138, | VL | Identifier 135, 137, 139, |
| | | 185, 187, 189, 216, 218, | | 184, 188, 217, 219, 237, |
| | | 220, 221,236, 246, 282, 284, | | 247, 283, 285, 287, 37, 39, |
| | | 286, 36, 38, 40, 84, 86, 88 | | 41, 87 in US20160168242 |
| | | in US20160168242 | | |
| IL3 alpha | VH | Identifier 22 in | VL | Identifier 27, 37 in |
| | | WO2008127735 | | WO2008127735 |
| IL4R | VH | Identifier 10, 11, 14, 15, 9 in | VL | Identifier 13, 7, 8 in |
| | | WO2009121847 | | WO2009121847 |
| ILIRAP | VH | Identifier 1, 10, 19, 8, 9 in | VL | Identifier 14, 15, 17, 18, 2, |
| | | WO2016020502; Identifier | | 20 in WO2016020502; |
| | | 120, 122, 124 in | | Identifier 121, 123, 125 in |
| | | WO2016179319A1 | | WO2016179319A1 |
| Integrin | VH | Identifier 3, 4, 5 in US | VL | Identifier 10, 11, 8, 9 in US |
| | | 20140161794 | | 20140161794 |
| KDR | VH | Identifier 20, 24, 26, 29, 31, | VL | Identifier 22 in |
| | | 33 in WO2003075840 | | WO2003075840 |
| KIR (Lirilumab) | VH | Identifier 3 in | VL | Identifier 5 in |
| | | US20150290316; 2371 | | US20150290316; Identifier |
| | | Identifier 1 in | | 2 in WO2014055648 |
| | | WO2014055648 | | |
| KIR2DL 1 | VH | Identifier 36 in | VL | Identifier 37 in |
| | | WO2016126213A1 | | WO2016126213A1 |
| KIR2DL 2/3 | VH | Identifier 36 in | VL | Identifier 37 in |
| | | WO2016126213A1 | | WO2016126213A1 |
| Klon43 | VH | Identifier 47 in | VL | Identifier 48 in |
| | | WO2016097231 | | WO2016097231 |
| KMA | VH | Identifier 22 in | VL | Identifier 2, 21 in |
| | | WO2016172703 A2 | | WO2016172703A2 |
| LAG3 | VH | Identifier 102, 106, 110, | VL | Identifier 32, 36, 40, 44, |
| | | 113, 122, 18, 30, 66, 70, 74, | | 48, 52, 56, 60, 84, 88, 92, |
| | | 78 in US20150259420; | | 96, 134, 34, 38, 42, 46, 50, |
| | | Identifier 100, 104, 108, 28, | | 54, 58, 60, 86, 90, 94, 98 in |
| | | 64, 68, 72, 76, 8, 80 in | | US20150259420; Identifier |
| | | US20150259420; Identifier | | 2 in WO2015042246 |
| | | 1 in WO2015042246 | | |
| Leukocytegen | VH | Identifier 25 in | — | — |
| A2 | | WO2010065962 A2 | | |
| Leukocytegen A | VH | Identifier 9 in | VL | Identifier 24 in |
| | | WO2010065962 A2 | | WO2010065962 A2 |
| LGR4 | VH | Identifier 12, 13, 5, 9 in | VL | Identifier 10, 11, 6 in |
| | | US20160046723 | | US20160046723 |
| LGR5 | VH | Identifier 10, 12, 16, 18, 20, | VL | Identifier 15, 19, 21, 23, |
| | | 22, 24, 26, 4 in | | 25, 3 in US20160102146 |
| | | US20160102146 | | |
| LHR | VH | Identifier 1, 2, 3, 4, 5, 6, 7, 8 | — | — |
| | | in WO2016160618A3 | | |
| Lymphotoxin | VH | Identifier 10, 12, 14, 16, 2 in | VL | Identifier 1, 15, 4, 6, 8 in |
| beta receptor | | WO2004002431 | | WO2004002431 |
| Lysyloxidase- | VH | Identifier 42, 44 in | VL | Identifier 43, 45 in |
| like 2 | | WO2011097513 | | WO2011097513 |
| Malignant | VH | Identifier 1 in | VL | Identifier 5 in |
| Variable | | WO2015133817A1 | | WO2015133817A1 |
| Receptor | | | | |
| MCAM | VH | Identifier 115, 116, 117, | VL | Identifier 109, 110, 111, |
| | | 118, 119, 157, 158, 159, | | 112, 121, 122, 123 in |
| | | 160, 161, 178, 179 in | | US20150259419; Identifier |
| | | US20150259419; Identifier | | 30, 40, 50, 60, 70, 71, 72 in |
| | | 35, 45, 55, 65, 77, 89 in | | US20150239980 |
| | | US20150239980; Identifier | | |
| | | 101, 102, 103, 104, 105, | | |
| | | 106, 107 in | | |
| | | US20150259419 | | |
| MCSF | VH | Identifier 102, 10, 14, 18, 2, | VL | Identifier 8, 32, 52, 60, 28, |
| | | 22, 26, 30, 34, 38, 46, 50, | | 36, 4, 44, 48, 56, 62, 12, |
| | | 54, 58, 6, 66, 70, 74, 78, 82, | | 16, 20, 24 in |
| | | 86, 90, 94, 98 in | | WO2005030124 |
| | | WO2005030124 | | |
| Mesothelin | VH | Identifier 1, 6 in | VL | Identifier 3, 5 |
| | | WO2015188141; Identifier | | WO2015188141; Identifier |
| | | 119, 50 in | | 1, 2, 3 in WO2013142034; |
| | | US20160333114A1; | | Identifier 11, 15, 19, 23, 27 |
| | | Identifier 5, 6 in | | in US20160229919A1; |
| | | WO2013142034; Identifier | | Identifier 120, 47, 49 in |
| | | 15, 2 in U.S. Pat. No. 9,416,190B2; | | US20160333114A1 |
| | | Identifier 13, 17, 21, 25, 29, | | |
| | | 9 in US20160229919A1 | | |
| MN | VH | Identifier 133, 135, 137, | VL | Identifier 134, 136, 138, |
| | | 139, 141, 143, 145, 147, | | 140, 142, 144, 146, 148, |
| | | 149, 151 in WO2007070538 | | 150, 152 in |
| | | | | WO2007070538 |
| MPER | VH | Identifier 13 in | VL | Identifier 12 in |
| | | US20160194375A1 | | US20160194375A1 |
| MUC1 | VH | Identifier 5 in | VL | Identifier 7 in |
| | | US20160130357; Identifier | | US20160130357; Identifier |
| | | 2, 14 in WO2013023162; | | 16, 7 in WO2013023162; |
| | | Identifier 15, 19, 23, 60, 64, | | Identifier 17, 21, 25, 62, |
| | | 68 in WO2015116753 | | 66, 70 in WO2015116753 |
| MUC16 | VH | Identifier 1, 21, 41, 81, 61 in | VL | Identifier 2, 22, 42, 62, 82 |
| | | WO2016149368; Identifier | | in WO2016149368 |
| | | 11, 4, 6, 8 in | | |
| | | US20130171152 | | |
| MUC1C ECD | VH | Identifier 15, 19, 23, 60, 64, | VL | Identifier 17, 21, 25, 62, |
| | | 68, 72 in | | 66, 70, 75 in |
| | | US20160340442A1 | | US20160340442A1 |
| MUCIN1 | VH | Identifier 101, 106, 109, | VL | Identifier 148, 158, 162, |
| | | 115, 119, 123, 127, 141, 15, | | 167, 170, 174, 184, 190, |
| | | 23, 28, 33, 39, 42, 47, 5, 57, | | 193, 203, 208, 211, 220, |
| | | 66, 70, 75, 80, 83, 87, 92 in | | 225, 229, 234, 242, 246, |
| | | EP3049812A2 | | 250, 255, 261, 270, 275, |
| | | | | 279, 283, 291, 297, 303, |
| | | | | 308, 315, 319, 323, 333, |
| | | | | 340 in EP3049812A2 |
| MVR | VH | Identifier 1 in | VL | Identifier 5 in |
| | | US20160257762A1 | | US20160257762A1 |
| N Glycan | VH | Identifier 7, 9 in | VL | Identifier 6, 8 in |
| | | US20160194375A1 | | US20160194375A1 N |
| NKG2A | VH | Identifier 32 in | VL | Identifier 33 in |
| | | WO2016126213A1; | | WO2016126213A1; |
| | | Identifier 2, 3, 4, 5, 6 in | | Identifier 7 in |
| | | WO2016041947 | | WO2016041947 |
| NKG2D | VH | Identifier 135, 137 in | VL | Identifier 134, 136 in |
| | | WO2016122701 | | WO2016122701 |
| NOTCH 1 | VH | Identifier 58 in | VL | Identifier 16, 20 in |
| | | US20160333114A1; | | WO2013074596; Identifier |
| | | Identifier 12 in | | 55, 57 in |
| | | WO2013074596 | | US20160333114A1 |
| NOTCH 2/3 | VH | Identifier 29 in | VL | Identifier 31 in |
| | | WO2013074596 | | WO2013074596 |
| Notum | VH | Identifier 56, 331 in | VL | Identifier 332, 58 in |
| | | WO2012027723 | | WO2012027723 |
| NYBR1 | VH | Identifier 19 in | VL | Identifier 18 in |
| | | US20160333422A1 | | US20160333422A1 |
| OlfmB | VH | Identifier 1 in | VL | Identifier 2 in |
| | | WO2015054441A1 | | WO2015054441A1 |
| Olfm13 | VH | Identifier 19, 3 in | VL | Identifier 20, 4 in |
| | | WO2015054441A1 | | WO2015054441A1 |
| Oncofetal | - | | VL | Identifier 1, 2, 7 in |
| fibronectin | | | | US20070202103A1 |
| Osteonectin | VH | Identifier 58 in | VL | Identifier 59 in |
| | | WO2016112870 | | WO2016112870 |
| OTK3 | VH | Identifier 17 in | VL | Identifier 18 in |
| | | WO2015158868 | | WO2015158868 |
| OX40 | VH | Identifier 101, 103, 105, | VL | Identifier 10, 45, 47, 49, 8 |
| | | 107, 109, 111, 113, 115, | | in U.S. Pat. No. 8,283,450; Identifier |
| | | 117, 119, 121, 123, 124, | | 11, 7 in U.S. Pat. No. 9,428,570; |
| | | 125, 17, 28, 318, 37, 48, 50, | | Identifier 116, 120, 122, |
| | | 58, 66,, 74, 85, 93, 95, 97, | | 30, 38, 49, 57, 65, 73, 84, |
| | | 99 in WO2016196228; | | 86, 94, 98 in |
| | | Identifier 31, 34, 36, 38, 40, | | WO2016196228; Identifier |
| | | 42, 44, 46, 48, 50, 53, 54, | | 24, 26, 27, 28, 30, 60, 8, |
| | | 55, 58, 59, 61 in | | 81, 82, 83, 84, 85, 86, 87, |
| | | US20150190506; Identifier | | 88, 89 in U.S. Pat. No. 8,748,585; |
| | | 33, 35, 37, 39, 41, 43, 45, | | Identifier 30, 32 in |
| | | 47, 49, 51, 53, 55, 57, 59, | | US20160137740; Identifier |
| | | 61, 63, 65, 67, 71 in | | 32, 35, 39, 41, 43, 45, 47, |
| | | US20160137740; Identifier | | 49, 51, 52, 56, 57, 62 in |
| | | 44, 46, 48, 7, 9 in | | US20150190506; Identifier |
| | | U.S. Pat. No. 8,283,450; Identifier 9, 15 | | 29, 37 in US20160137740 |
| | | in U.S. Pat. No. 9,428,570; Identifier | | |
| | | 19, 21, 22, 23, 29, 58, 59, 7, | | |
| | | 77, 78, 79, 80 in | | |
| | | U.S. Pat. No. 8,748,585 | | |
| PD1 | VH | Identifier 19 in | VL | Identifier 2, 39, 7, 8, 9 in |
| | | US20150290316; Identifier | | US 20160159905; |
| | | 25, 26, 27, 28, 29 in | | Identifier 21 in |
| | | US20130291136; Identifier | | US20150290316; Identifier |
| | | 29, 3, 38 in US | | 30, 31, 32, 33 in |
| | | 20160159905; Identifier 38, | | US20130291136; Identifier |
| | | 50 in WO2015112900; | | 42, 46, 54 in |
| | | Identifier 4, 4, 6 in US | | WO2015112900; Identifier |
| | | 20160159905; Identifier 82, | | 58, 62, 66, 70, 74, 78 in |
| | | 86 in WO2015112900; | | WO2015112900; Identifier |
| | | Identifier 17 in | | 18 in WO2014055648; |
| | | WO2014055648; Identifier | | Identifier 5 in |
| | | 4 in WO2016040892; | | WO2016040892; Identifier |
| | | Identifier 12 in | | 13 in US20150190506 |
| | | US20150190506 | | |
| PDK1 | VH | Identifier 2 in | VL | Identifier 9 in |
| | | WO2016090365 | | WO2016090365 |
| PDI (Nivolumab) | VH | Identifier 2 in | VL | Identifier 11 in |
| | | WO2016040892; Identifier | | US20150190506 |
| | | 10 in US20150190506 | | |
| PDL1 | VH | Identifier 10, 32, 8 in | VL | Identifier 22, 26, 34, 42, |
| | | US20160319022; Identifier | | 58, 66, 74, 82, 86 in |
| | | 18, 30, 38, 46, 50, 54, 62, | | WO2016061142; Identifier |
| | | 70, 78 in WO2016061142; | | 30, 8, 9 in |
| | | Identifier 29, 7 in | | US20150190506; Identifier |
| | | US20150190506; Identifier | | 7, 9 in US20160319022; |
| | | 16, 18, 197, 247, 248, 250, | | Identifier 17, 22, 24, 249, |
| | | 251, 252, 253, 254, 255, | | 26, 28, 309, 311, 313, 320, |
| | | 256, 257, 258, 259, 260, 30, | | 325, 34, 340, 357, 359, 36, |
| | | 308, 310, 312, 319, 32, 324, | | 42, 44, 58, 60, 66, 68, 74, |
| | | 339, 356, 38, 40, 46, 48, 50, | | 76, 8, 82, 84, 86, 88 in |
| | | 52, 54, 6, 62, 70, 72, 78, 80, | | US20160108123 |
| | | 91, 96 in US20160108123; | | |
| | | Identifier 358, 56, 64 in | | |
| | | US20160108123 | | |
| PDL2 | VH | Identifier 43, 44, 56, 46 in | VL | Identifier 47, 48, 49, 50, 51 |
| | | US20130291136 | | in US20130291136 |
| PG16 | VH | Identifier 13 in | VL | Identifier 12 in |
| | | EP3074419A2 | | EP3074419A2 |
| PG9 | VH | Identifier 11 in | VL | Identifier 10 in |
| | | EP3074419A2 | | EP3074419A2 |
| PGT1 | VH | Identifier 15 in EP307441 | — | — |
| PGT2 | VH | Identifier 17 in | VL | Identifier 16 in |
| | | EP3074419A2 | | EP3074419A2 |
| PGT3 | VH | Identifier 19 in | VL | Identifier 18 in |
| | | EP3074419A2 | | EP3074419A2 |
| PGT4 | VH | Identifier 21 in | VL | Identifier 20 in |
| | | EP3074419A2 | | EP3074419A2 |
| PGT5 | VH | Identifier 23 in | VL | Identifier 22 in |
| | | EP3074419A2 | | EP3074419A2 |
| PRAME | VH | Identifier 50, 52, 54, 56, 58, | VL | Identifier 49, 51, 53, 55, |
| | | 60, 62 in | | 57, 59, 61 in |
| | | WO2016191246A2 | | WO2016191246A2 |
| PRP | VH | Identifier 42 in | VL | Identifier 39, 41 in |
| | | US20160333114A1 | | US20160333114A1 |
| PSMA | VH | Identifier 43 in | VL | Identifier 44 in |
| | | WO2016097231 | | WO2016097231; Identifier |
| | | | | 44 in WO2016097231 |
| PTK7 | VH | Identifier 1, 25, 49 in | VL | Identifier 20, 22, 24, 26, |
| | | US20150315293; Identifier | | 28, 30, 32, 34, 36, 38, 40, |
| | | 21, 23, 25, 27, 29, 31, 33, | | 42, 44, 46, 48, 50, 52, 54, |
| | | 35, 37, 39, 41, 43, 45, 47, | | 56, 58, 60, 62, 64, 66, 68 in |
| | | 49, 51, 53, 55, 57, 59, 61, | | WO2012112943A1; |
| | | 63, 65, 67, 69 in | | Identifier 15, 39, 63 in |
| | | WO2012112943A1 | | US20150315293 |
| RAS | VH | Identifier 17, 47, 57, 67, 7, | VL | Identifier 19, 49, 59, 69, |
| | | 77 in WO2016154047 | | 79, 9 in WO2016154047 |
| RHAMM | VH | Identifier 4 in | — | — |
| | | US20020127227A1 | | |
| RHAMM | VH | Identifier 2 in | VL | Identifier 4 in |
| antagonist body | | WO2000029447 | | WO2000029447 |
| Rituximab | VH | Identifier 66 in | VL | Identifier 63, 65 in |
| | | US20160333114A1 | | US20160333114A1 |
| ROR1 | VH | Identifier 12, 20, 28, 36, 44, | VL | Identifier 16, 24, 32, 40, |
| | | 60, 68 WO2016016343A1; | | 56, 64, 72, 36, 62, 23, 49, |
| | | Identifier 57, 19, 31, 45, 53, | | 58 WO2016016343A1; |
| | | 71 in WO2016016344A1; | | Identifier 86, 88, 90 in |
| | | Identifier 85, 87, 89 in | | WO2016120216; Identifier |
| | | WO2016120216; Identifier | | 126, 127, 234, 235, 236, |
| | | 122, 125, 175, 176, 179, | | 237, 238, 240, 241, 242, |
| | | 180, 181, 182, 183, 184, | | 243, 244, 245, 246, 247, |
| | | 185, 186, 187, 188, 189, | | 248 in US20160208018A1; |
| | | 190, 191, 192, 193, 194, | | Identifier 56 in |
| | | 195, 196, 197, 197, 199, | | EP3083671A1; Identifier |
| | | 200, 201, 202, 203, 204, | | 103, 111, 127, 135, 143, |
| | | 205, 206, 207, 208, 209 in | | 15, 151, 159, 167, 175, |
| | | US20160208018A1; | | 183, 191, 199, 207, 215, |
| | | Identifier 55 in | | 223, 23, 231, 239, 247, |
| | | EP3083671A1; Identifier | | 255, 263, 271, 279, 287, |
| | | 104, 112, 120, 128, 152, 16, | | 295, 303, 31, 311, 319, |
| | | 160, 168, 176, 184, 192, | | 327, 335, 343, 351, 359, |
| | | 200, 208, 216, 224, 232, 24, | | 39, 47, 55, 63, 7, 71, 79, |
| | | 240, 248, 256, 264, 272, | | 87, 95 in |
| | | 280, 288, 296, 304, 312, 32, | | WO2016187216A1 |
| | | 320, 336, 344, 352, 360, 40, | | |
| | | 48, 56, 64, 72, 8, 80, 88 in | | |
| | | WO2016187216A1 | | |
| SEMAPHORIN | VH | Identifier 10, 25, 9 in | VL | Identifier 17, 18, 29 in |
| 4D | | US20160115240A1 | | US20160115240A1 |
| TAG72 | VH | Identifier 115 in | VL | Identifier 116 in |
| | | US20160333114A1 | | US20160333114A1 |
| TCR | VH | Identifier 133 in | VL | Identifier 132 in |
| | | WO2016122701 | | WO2016122701 |
| TEM8 | VH | Identifier 1, 3, 5, 7 in | VL | Identifier 4, 6, 8 in |
| | | US20160264662A1 | | US20160264662A1 |
| Tie | VH | Identifier 723 in | VL | Identifier 724 in |
| | | US20060057138A1 | | US20060057138A1 |
| TIGIT | VH | Identifier 10, 11, 12, 124, | VL | Identifier 130, 131, 132, |
| | | 125, 126, 127, 128, 129, 13, | | 133, 137, 139, 145, 146, |
| | | 136, 138, 14, 143, 144, 149, | | 151, 152, 25, 26, 27, 28, |
| | | 15, 150, 16, 17, 18, 19, 20, | | 29, 30, 50, 51, 52, 64, 95, 8 |
| | | 21, 22, 23, 24, 37, 38, 39, | | in US20160355589 |
| | | 40, 41, 42, 43, 44, 45, 46, | | |
| | | 47, 63, 94, 7, 9 in | | |
| | | US20160355589 | | |
| TIM3 | VH | Identifier 102, 112, 12, 2, | — | — |
| | | 22, 32, 42, 52, 62, 72, 82, 91 | | |
| | | in US20150086574; | | |
| | | Identifier 82 in | | |
| | | WO2013006490; Identifier | | |
| | | 13, 21, 29, 37, 45, 5, 53, 61, | | |
| | | 69, 77, 85, 93 in | | |
| | | WO2016179319A1; | | |
| | | Identifier 7 in | | |
| | | WO2013006490; Identifier | | |
| | | 107, 117, 17, 27, 37, 47, 57, | | |
| | | 67, 7, 77, 87, 97 in | | |
| | | US20150086574; Identifier | | |
| | | 17, 25, 33, 41, 49, 57, 65, | | |
| | | 73, 81, 89, 9, 97 in | | |
| | | WO2016179319A1 | | |
| Tissue factor | VH | Identifier 10, 19, 23, 27, 29, | VL | Identifier 25. 31 in |
| | | 6 in WO2004094475; | | US20040229301A1; |
| | | Identifier 38 in | | Identifier 12, 21, 25, 31, 8 |
| | | US20160333114A1 | | in WO2004094475; |
| | | | | Identifier 35, 37 in |
| | | | | US20160333114A1 |
| Tn Glycopeptide | VH | Identifier 19, 20 in | — | — |
| | | WO2015120180 | | |
| TRBC1 | VH | Identifier 1 in | VL | Identifier 2 in |
| | | WO2015132598 | | WO2015132598 |
| Trophoblast | VH | Identifier 17, 13, 15, 11 in | VL | Identifier 18, 12, 14, 16 in |
| Glycoprotein | | WO2016034666A1 | | WO2016034666A1 |
| 5T4 | | | | |
| Upar | VH | Identifier 72 in | VL | Identifier 71, 73 in |
| | | US20160333114A1 | | US20160333114A1 |
| V2 | VH | Identifier 11 in | — | — |
| | | US20160194375A1 | | |
| VEGF | VH | Identifier 4, 8, in | VL | Identifier 2, 6 in |
| | | WO2000034337; Identifier | | WO2000034337; Identifier |
| | | 12, 20, 4, 44 in | | 9 in US20030175276A1; |
| | | WO2006012688A1; | | Identifier 11, 19, 27, 28, 3, |
| | | Identifier 7 in | | 43 in WO2006012688A1; |
| | | US20030175276A1; | | Identifier 160, 161, 162, |
| | | Identifier 152, 153, 154, | | 163, 164, 165, 166, 167 in |
| | | 155, 156, 157, 158, 159 in | | US20160090427 |
| | | US20160090427 | | |
| VEGFR2 | VH | Identifier 100, 101, 102, | VL | Identifier 107, 108, 109, |
| | | 103, 114, 115, 116, 117, | | 110, 111, 112, 113, 86, 87, |
| | | 118, 119, 120, 121, 122, | | 88, 89, 90, 91, 92, 93, 94 in |
| | | 123, 124, 95, 96, 97, 98, 99 | | WO2017004254 |
| | | in WO2017004254 | | |
| VISTA | VH | Identifier 37, 38, 39, 40 in | VL | Identifier 41, 42, 43, 44, 45 |
| | | WO2015097536 | | in WO2015097536 |
| VMS2 | VH | FIG. 1 in WO2000058363 | — | — |
| WT1/HLA | VH | Identifier 104, 111, 128, 14, | VL | Identifier 106, 112, 130, |
| Bispecific | | 32, 50, 68, 86 in | | 34, 52, 70, 88 in |
| | | WO2015070061 | | WO2015070061 |

Table 2 provides exemplary antigen targets. The antigen-binding domain may comprise an scFv derived from an antibody or antibody fragment that binds to an antigen target such as those described in the cited publications, the contents of each publication are incorporated herein by reference in their entirety for all purposes.

TABLE 2

| Antigen Target | Examples of Source |
|---|---|
| Activated alpha- | Identifier 8, 2, 4 in US20090117096A1 |
| v-beta-3 integrin | |
| receptor | |
| Adalimumab | Identifier 41 in US20160208021; Identifier 41 in WO2016112870 |
| ALK | Identifier 17, 18, 19, 20, 21, 22, 23 in WO2015069922; Identifier 17, 18, |
| | 19, 20, 21, 22, 23, 24 in US20160280798A1; Identifier 24 in |
| | WO23015069922 |
| B7H3 | Identifier 99, 100, 101, 102, 103, 104, 102, 17, 18, 19, 20, 21, 22, 23, 24, |
| | 25, 26, 27, 87, 88, 89, 90, 91, 92, 94, 95, 96, 97, 98 in WO2016033225 |
| B7H4 | Identifier 1, 2, 3, 4 in WO2013067492; Identifier 1 in U.S. Pat. No. 9,422,351B2 |
| BCMA | Identifier 152, 158, 176, 185, 188, 200, 212, 218, 224, 284, 290, 296, 302, |
| | 314, 326, 344, 129, 130, 131, 132, 133, 134, 135, 136, 138, 139, 140, 141, |
| | 142, 143, 144, 145, 146, 147, 148, 149, 263, 264, 265, 266, 271, 273, 39, |
| | 40, 41, 42, 43, 44, 45, 46, 47, 48, 49, 50, 51, 52, 53, 64, 129, 130, 131, |
| | 132, 133, 134, 135, 136, 137, 138, 139, 140, 141, 142, 143, 144, 145, 146, |
| | 147, 148, 149, 263, 264, 265, 266, 39, 40, 41, 42, 43, 44, 45, 46, 47, 48, |
| | 49, 50, 51, 52, 53 in WO2016014565; Identifier 214, 215, 216, 217, 218, |
| | 219, 220, 221, 222, 223, 224, 225, 226, 227, 228, 229, 230, 231, 232, 233, |
| | 234, 235, 236, 237, 238, 239, 240, 241, 242, 243, 244, 245, 246, 247, 248, |
| | 249, 251 in US20160311907A1 |
| CCR4 | Identifier 7, 9 in WO2015191997 |
| CD123 | Identifier 157, 158, 159, 160, 184, 185, 186, 187, 188, 189, 190, 191, 192, |
| | 193, 194, 195, 196, 197, 198, 199, 200, 201, 202, 203, 204, 205, 206, 207, |
| | 208, 209, 210, 211, 212, 213, 214, 215, 478, 480, 483, 485 in |
| | WO2016028896; Identifier 36 in WO2015092024A2; Identifier 57 in |
| | WO2016115482A1; Identifier 36 in EP3083691A2; Identifier 157 in |
| | US20160311907A1 |
| CD124 | Identifier 158 in US20160311907A1 |
| CD125 | Identifier 159 in US20160311907A1 |
| CD126 | Identifier 160 in US20160311907A1 |
| CD127 | Identifier 161 in US20160311907A1 |
| CD128 | Identifier 162 in US20160311907A1 |
| CD129 | Identifier 163 in US20160311907A1 |
| CD130 | Identifier 164 in US20160311907A1 |
| CD131 | Identifier 165 in US20160311907A1 |
| CD138 | Identifier 36 in WO2016130598A1 |
| CD19 | Identifier 53, 54, 37 in EP3083671A1; Identifier 1, 10, 11, 12, 2 in |
| | WO2015157252; Identifier 10, 2, 206, 207, 208, 209, 210, 211, 213, 214, |
| | 215, 216, 217, 218, 219, 220, 221, 222, 223, 224, 225, 4, 45, 47, 49, 51, |
| | 53, 55, 57, 51, 53, 55, 57, 59, 6, 8, 87 in WO2016033570; Identifier 3, 4, |
| | 5, 59, 6, 7, 8, 9 in WO2015157252; 5754 Identifier 5 in |
| | WO2015155341A1; Identifier 7 in WO2014184143; Identifier 9 in |
| | WO2016139487; Identifier 10, 2, 206, 207, 208, 209, 210, 211, 212, 213, |
| | 214, 215, 216, 217, 218, 219, 220, 221, 222, 223, 224, 225, 4, 45, 47, 49, |
| | 51, 53, 55, 57, 59, 6, 8, 87, 89 in US20160152723; Identifier 32, 35, 38 in |
| | EP3083691A2; Identifier 174 in WO2016115482A1; Identifier 20 in |
| | WO2012079000; Identifier 32, 33, 35, 38 in WO2015092024A2; |
| | Identifier 39, 40, 41, 42, 43, 44, 45, 46, 47, 48, 49, 50, 51 in |
| | WO2016109410; Identifier 5, 6 in WO2015155341A1; Identifier 7, 9 in |
| | US20160145337A1; Identifier 20 in U.S. Pat. No. 9,499,629B2; Identifier 73 in |
| | WO2016164580; Identifier 10, 2, 206, 207, 209, 210, 212, 216, 218, 219, |
| | 220, 221, 222, 223, 224, 225, 4, 45, 47, 49, 51, 53, 55, 57, 59, 6, 8, 87, 89 |
| | in US20160152723; Identifier 5 in WO2016055551 |
| CD19/CD22 | Identifier 1303, 1307 in WO2016164731A2 |
| Bispecific | |
| CD20 | Identifier 691 in WO2016164731A100; Identifier 692 in |
| | WO2016164731A101; Identifier 693 in WO2016164731A102; Identifier |
| | 694 in WO2016164731A103; Identifier 695 in WO2016164731A104; |
| | Identifier 696 in WO2016164731A105; Identifier 175 in |
| | WO2016115482A1 |
| CD22 | Identifier 5, 6 in WO2013059593; Identifier 9 in US20150299317; |
| | Identifier 131, 132, 133, 134, 135, 136, 137, 138, 139, 140, 203, 209, 215, |
| | 221, 227, 232, 238, 244, 250, 256, 262, 268, 274, 280, 286, 292, 298, 304, |
| | 310, 316, 322, 328, 334, 340, 346, 353, 358, 364, 370, 376, 383, 388, 394, |
| | 400, 406, 412, 418, 423 in WO2016164731A2 |
| CD276 | Identifier 10, 19, 28 in US20160053017 |
| CD3 | Identifier 46, 47 in WO2015153912A1 |
| CD30 | Identifier 20 in WO2016116035A1; Identifier 2 in US20160200824A1 |
| CD33 | Identifier 262, 263, 264, 265, 266, 267, 268, 39, 40, 41, 42, 43, 44, 45, 46, |
| | 47 in WO2016014576; Identifier 37 in WO2015092024 A2; Identifier 37 |
| | in EP3083691A2; Identifier 153, 154, 155, 156, 157, 158, 159, 160, 161, |
| | 162, 163 in WO2016115482A1 |
| CD33/CD3s | Identifier 33, 34, 84 in WO2014144722A2 |
| bispecific | |
| CD37 | Identifier 21, 22 in US20170000900 |
| CD44 | Identifier 17 in WO2016042461A1 |
| CD46 | Identifier 1-42 in WO2016040683 |
| CD5 | Identifier 16 in WO2016138491 |
| CD79b | Identifier 33 in US20160208021 |
| CEA | Identifier 1 in US20160303166A1; Identifier 22 in US20140242701A1 |
| Cetuximab | Identifier 37 in WO2016112870; Identifier 37 in US20160208021 |
| Claudin | Identifier 11, 5, 7, 9 in WO2016073649A1; Identifier 17 in |
| | WO2014179759A1 |
| Claudin6 | Identifier 164 in WO2016115482A1 |
| Claudin7 | Identifier 165 in WO2016115482A1 |
| Claudin8 | Identifier 166 in WO2016115482A1 |
| CLDN6 | Identifier 2 in WO2016150400 |
| CLDN7 | Identifier 4 in WO2016150400 |
| CLDN8 | Identifier 6 in WO2016150400 |
| CLL1 | Identifier 39, 40, 41, 42, 43, 44, 45, 46, 48, 49, 50, 51 in WO2016014535; |
| | Identifier 200, 201, 202, 203, 204, 205, 206, 207, 208, 209, 210, 211, 212, |
| | 213 in US20160311907A1 |
| CMet | Identifier 11, 12, 13, 14, 15, 16, 17, 18, 19, 2, 21, 22, 23, 25, 26, 27, 28, 3, |
| | 30, 31, 33, 34, 35, 36, 37, 38, 39, 4; 40, 41, 42, 43, 44, 48, 49, 5, 50, 51, |
| | 52, 53, 54, 55, 56, 57, 58, 6, 60, 7, 9, 29 in US20040166544; Identifier 26, |
| | 27, 28, 29, 30, 32 in US20150299326; Identifier 32 in US20130034559 |
| CS1 | Identifier 1 of WO2016090369; Identifier 17 in WO2014179759A1 |
| CSPG4 | Identifier 2 in WO2015080981; Identifier 2 in EP3074025A1 |
| CXCR4 | Identifier 83, 85, 86, 89 in US20110020218 |
| E7MC | Identifier 223, 224, 225, 226, 227, 228, 229, 230, 231, 232 in |
| | WO2016182957A1 |
| EGFR | Identifier 11, 38, 41, 44, 47, 50, 53, 56, 59, 62, 65, 68, 71, 74, 77, 80, 83, |
| | 88, 91, 94 in WO2014130657 |
| EGFR VIII | Identifier 5 in US20140037628; Identifier 174 in US20160311907A1; |
| | Identifier 38 in U.S. Pat. No. 9,394,368B2; Identifier 5 in US20160200819A1 |
| END 0180 | Identifier 6 in WO2013098813 |
| ERBB2 | Identifier 26, 27 in US20110059076A1; Identifier 1, 2 in U.S. Pat. No. 7,244,826 |
| ESKAVT | Identifier 173 in WO2016115482A1 |
| FcRL | Identifier 11, 15, 19, 23, 27, 31, 35, 39, 3, 43, 7, 594, 596, 598, 600, 602, |
| | 604, 606, 608, 610, 612, 614, 616, 618, 620, 622, 624, 626, 628, 630, 632, |
| | 634, 636, 638, 640, 642, 644, 646, 648, 652, 654, 656; 658, 660, 662, 664, |
| | 666, 668, 670, 672, 674, 676, 680, 682, 684, 686, 688, 690, 692, 694, 696, |
| | 700, 702, 704, 706, 708, 710, 712, 714, 716, 718, 720, 722, 724, 726, 728, |
| | 730, 732, 734, 736, 738, 740, 742, 744, 746, 748, 750, 752, 754, 756, 758, |
| | 760, 762, 764, 766, 768, 770, 772, 774, 776, 778, 780, 782, 784, 786, 788, |
| | 790, 792, 794, 796, 798, 800, 802, 804, 806, 808, 810, 812, 814, 816, 818, |
| | 820, 822, 824, 826, 828, 830, 832, 834, 836, 838, 840, 842, 844; 846, 848, |
| | 850, 852, 854, 856, 858, 860, 862, 864, 866, 868, 870, 874, 876, 878, 880, |
| | 882, 884, 886, 888, 890, 892, 894, 896, 650, 678 in WO2016090337 |
| Folate receptor | Identifier 15 in US20170002072A1 |
| Folate receptor | Identifier 15, 23 in WO2012099973 |
| alpha | |
| FOLRI/CD3s | Identifier 90 in WO2014144722A2 |
| bispecific | |
| GCN4 | Identifier 165, 166, 167, 168, 169, 170 in WO2016168773 A3 |
| GD2 | Identifier 19, 20, 21, in WO2016134284; Identifier 8 in WO2015132604 |
| GPC3 | Identifier 1 in WO2016049459; Identifier 12 in US20160208015A1 |
| GPC4 | Identifier 24 in WO2016049459 |
| GPRC5D | Identifier 100, 101, 102, 103, 104, 105, 106, 107, 108, 109, 110, 111, 112, |
| | 113, 114, 115, 116, 117, 118, 119, 120, 121, 122, 123,301, 313, 325, 337, |
| | 349, 361, 373, 385 in WO2016090312 |
| HER2/CD3 | Identifier 9 in WO2014144722 A2 |
| Human CD79b | Identifier 33 in WO2016112870 |
| Human collagen | Identifier 34 in WO2016112870 |
| VII | |
| IL4 | Identifier 17, 16 in WO2009121847 |
| Integrin bivalent | Identifier 2, 1 in WO2009070753 |
| Ipilimumab | Identifier 39 in US20160208021; Identifier 39 in WO2016112870 |
| Mec/CD3s | Identifier 78 in WO2014144722A2 |
| bispecific | |
| Mesothelin | Identifier 7 WO2015188141; Identifier NO 47, 46, 57, 48, 49, 50, 51, 53, |
| | 54, 55, 56, 58, 59, 62, 64, 65, 66, 67, 68, 69, 70, 52, 60, 61, 63 in |
| | WO2016090034; Identifier 10, 11, 12 in WO2013142034; Identifier 11 in |
| | WO2013063419 |
| MUC1 | Identifier 15 in US20160130357 |
| MUC2 | Identifier 17 in US20160130357 |
| MUC3 | Identifier 15 in US20160130357 |
| MUC4 | Identifier 17 in US20160130357 |
| Nivolumab | Identifier 38 in US20160208021; Identifier 38 in WO2016112870 |
| NYBR1 | Identifier 21 in US20160333422A1; Identifier 21, 18, 19 in |
| | WO2015112830 |
| O-acetylated GD2 | Identifier 29, 31 in US20150140023 |
| ganglioside | |
| OX40 | Identifier 33 in US20150190506 |
| PD1 | Identifier 39, 40, 41, 42, 43, 44, 45, 46, 47, 48, 49, 50, 51, 52, 53, 54, 55, |
| | 56, 57, 58, 59, 60, 61 in US20160311917A1 |
| PDK1 | Identifier 15 in WO2016090365 |
| PDL1 nanobody | Identifier 22, 23, 24, 25, 26, 27 in US20110129458 |
| PDL2 nanobody | Identifier 28, 29, 30, 31, 32, 33 in US20110129458 |
| 6462 | |
| PRAME | Identifier 63, 64, 65, 66, 67, 68, 69 in WO2016191246A2 |
| PSMA | Identifier 19, 21, 30, 31, 34, 35 in WO2012145714 |
| PSMA diabody | Identifier 12, 13, 14, 15 in WO2011069019 |
| Radiation | Identifier 22, 24 in WO2005042780A1 |
| inducible | |
| neoantigen | |
| Ranibizumab | Identifier 40 in US20160208021; Identifier 40 in WO2016112870 |
| RAS | Identifier 81 in WO2016154047 |
| Rituximab | Identifier 36 in US20160208021; Identifier 36 in WO2016112870 |
| RORI | Identifier 34 in EP3083691A2; Identifier 249, 250 251, 252, 253, 254, |
| | 255, 256, 257, 258, 259, 260, 261, 262, 263, 264, 265, 266, 267, 268 in |
| | US20160208018A1; Identifier 57 in EP3083671A1; Identifier 1, 2 in |
| | US20160304619A1; Identifier 34 in WO2015092024A2 |
| Teplizumab | Identifier 42 in WO2016112870 |
| Teplizumab | Identifier 42 in US20160208021 |
| (mutated) | |
| TOSO | Identifier 2 in EP3098237A1 |
| Trastuzumab | Identifier 35 in US20160208021; Identifier 35 in WO2016112870 |
| TRBC1 | Identifier 13, 14, 15, 16, 17, 18, 19, 20, 21, 22, 3 in WO2015132598 |
| TRBC2 | Identifier 23, 24, 25, 26, 27, 28, 29, 30, 31, 32 in WO2015132598 |
| TSLPR | Identifier 1, 2 in US20160311910A1; Identifier 1, 2 in WO2015084513 |
| VEGF | Identifier 168, 169, 170, 171, 172, 173, 174, 175 in US20160090427; |
| | Identifier 498, 500, 502, 504, 506, 508 in US20110177074A1 |
| VEGFR2 | Identifier 1, 2 in US20120213783 |
| WT1/HLA | Identifier 108, 113, 18, 36, 54, 72, 90 in WO2015070061 |
| bispecific | |

Table 3 provides exemplary antigen targets. The antigen-binding domain may comprise an antigen-binding domain derived from a CAR that binds to an antigen target, such as those described in the cited publications, the contents of each publication are incorporated herein by reference in their entirety for all purposes.

TABLE 3

| Antigen Target | Examples of Source |
|---|---|
| Acid/base leucine | Identifier 34, 35 in WO2016124930 |
| zipper domains | |
| ALK | Identifier 43, 44, 45, 46, 47, 48, 49, 50, 51, 52, 53, 54, 55, 56, 57, 58, |
| | 59, 60, 61, 62, 63, 64, 65, 66, 67, 68, 69, 70, 71, 72, 73, 74, 75, 76, |
| | 77, 78, 79, 80, 81, 82, 84, 85, 86, 87, 88, 89, 90 in WO2015069922 |
| APRIL-based CAR | Identifier 53 in US20160296562A1; Identifier 52 in |
| | US20160296562A1 |
| BCMA | Identifier 180, 162, 168, 174, 144, 150, 186, 192, 198, 204, 210, 156, |
| | 216, 222, 228, 234, 240, 246, 252, 258, 264, 270, 276 330, 282, 300, |
| | 306, 336, 354, 288, 312, 294, 342, 324, 318, 348 in |
| | WO2016168595A1; Identifier 48, 49, 50, 51, 52, 53, 54, 55, 56, 57, |
| | 58, 59, 19, 20, 21, 22, 23, 24, 25, 26, 27, 28, 29, 30, 31, 32, 33, 34, |
| | 35, 36, 37, 38, 39, 40, 41, 42 in WO2015158671A1; Identifier 124, |
| | 114, 115, 116, 117, 118, 119, 120, 121, 122, 123, 125, 126, 127, 128, |
| | 234, 235, 236, 237, 238, 239, 240, 241, 242, 243, 244, 245, 246, 247, |
| | 248, 249, 250, 251, 252, 253, 254, 267, 268, 269, 270 in |
| | WO2016014565; Identifier 1, 2, 3, 4, 5, 20 in WO2015052538; |
| | Identifier 1, 2, 3, 4, 5, 6 in US20160237139A1; Identifier 9 in |
| | WO2016094304 A3; Identifier 4, 5, 6, 8, 9, 10, 11, 12 in |
| | WO2013154760; Identifier 15, 16, 17, 18, 19, 20, 21, 22, 23, 24, 25, |
| | 26, 27, 28, 29, 71, 73 in WO2016014789; Identifier 125, 126, 127, |
| | 128, 129, 130, 131, 132, 133, 134, 135, 136, 137, 138, 139, 140, 141, |
| | 145, 146, 147, 148, 149, 150 in WO2016120216; Identifier 102, 106, |
| | 107, 108, 109, 110, 111, 112, 129, 130, 131, 132, 133, 134, 135, 136, |
| | 113, 114, 115, 116, 117, 118, 101, 100, 137, 119, 120, 121, 122, 123, |
| | 124, 125, 126, 127, 128, 103, 104, 105, 213 in WO2016097231 |
| CAR and gate (CD19 | Identifier 2 in US20160296562 |
| and CD33) CD148 | |
| phosphatase | |
| CAR and gate (CD19 | Identifier 43 in US20160296562 |
| and CD5) | |
| CAR and gate (CD19 | Identifier 45 in US20160296562 |
| and EGFR VIII) | |
| CAR and gate (CD19 | Identifier 41 in US20160296562 |
| and GD2) | |
| CAR and gate (CD19 | Identifier 3 in US20160296562 |
| or CD33) CD45 | |
| phosphatase | |
| CAR and not gate | Identifier 4, 5 in US20160296562 |
| (CD19 and not CD33) | |
| CAR and not gate | Identifier 6 in US20160296562A1 |
| (CD19 and not CD33) | |
| CAR and not gate 1 | Identifier 48 in US20160296562 |
| CAR and not gate 2 | Identifier 49 in US20160296562 |
| CAR and not gate 3 | Identifier 50 in US20160296562 |
| CAR or gate (DC19 or | Identifier 1 in US20160296562 |
| DC33) | |
| CAT19 CAR with | Identifier 12 in WO2016139487 |
| CD28 zeta | |
| endodomain | |
| CAT19 CAR with | Identifier 11 in WO2016139487 |
| OX40 zeta | |
| endodomain | |
| CAT19, campana | Identifier 10 in WO2016139487 |
| architecture | |
| CD123 | Identifier 69 in WO2016142532; Identifier 23, 24, 25, 26, 27, 28, 29, |
| | 30, 31, 32, 33, 34, 35, 36, 37, 38, 39, 40, 41, 42, 44, 45, 46, 47, 48 in |
| | WO2015140268A1; Identifier 9, 10, 11, 12 in US20140271582; |
| | Identifier 56, 57, 58, 59, 60, 61 in WO2016097231; Identifier 98, 99, |
| | 100, 101, 125, 126, 127, 128, 129, 130, 131, 132, 133, 134, 135, 136, |
| | 137, 138, 139, 140, 141, 142, 143, 144, 145, 146, 147, 148, 149, 150, |
| | 151, 152, 153, 154, 155, 156 in WO2016028896; Identifier 31, 32, |
| | 33, 34, 35, 36, 37, 38, 39, 40, 41, 42, 43, 44, 45, 46, 47, 48, 49, 50, |
| | 51, 52, 53, 54, 55, 56, 57, 58, 59, 60, 61, 62, 63, 64, 65, 66, 67, 68, |
| | 69, 70, 71, 72, 73, 74, 75, 76, 77, 78, 79, 80, 81, 82, 83, 84, 85, 86, |
| | 87, 88, 89, 90, 91, 92, 93, 94, 95, 96, 97, 98, 99, 100, 101, 102, 103, |
| | 104, 105, 106, 107, 108, 109, 110, 111, 112, 113, 114, 115, 116, 117, |
| | 118, 119, 120, 121, 122, 123, 124, 125, 126, 127, 128, 129, 130, 131, |
| | 132, 133, 134, 135, 136, 137, 138, 139, 140, 141, 142, 143, 144, 145, |
| | 146, 147, 148, 149, 150, 151, 152, 153, 154, 155, 156, 157, 158, 159, |
| | 160, 172, 173, 174, 175, 176, 177, 178, 179, 180, 181, 182, 183, 184, |
| | 185, 186, 187, 188, 189, 190, 191, 193, 194, 195, 196, 197 in |
| | WO2016120220; Identifier 1, 2, 3, 4, 5, 6, 7, 8, 9, 10, 142 in |
| | WO2016120216 |
| CD19 | Identifier 12 in U.S. Pat. No. 9,499,629B2; Identifier 24 in US20160333108A1; |
| | Identifier 25, 29 in US20160333108A1; Identifier 27 in |
| | US20160333108A1; Identifier 1 in EP2997134A4; Identifier 19, 20 |
| | in EP3071687A1; Identifier 181 in WO2016168773A3; Identifier 2 |
| | in WO2015157399A9; Identifier 56, 62 in WO2016174409A1; |
| | Identifier 145, 293, 294, 295, 296, 297, 298 in WO2016179319A1; |
| | Identifier 73 in WO2013176915A1; Identifier 73 in |
| | WO2013176916A1; Identifier 73 in US20130315884A1; Identifier |
| | 73 in US20140134142A1; Identifier 73 in US20150017136A1; |
| | Identifier 73 in US20150203817A1; Identifier 73 in |
| | US20160120905A1; Identifier 73 in US20160120906A1; Identifier |
| | 8, 5 in WO2015124715; Identifier 73 in WO2014184744; Identifier |
| | 73 in WO2014184741; Identifier 14, 15 in US20160145337A1; |
| | Identifier 14, 15 in WO2014184143; Identifier 15, 16 in |
| | WO2015075175; Identifier 16 in US20160145337A1; Identifier 16 in |
| | WO2014184143; Identifier 12 in WO2012079000; Identifier 31, 32, |
| | 33, 34, 35, 36, 37, 38, 39, 40, 41, 42, 58 in WO2016164580; |
| | Identifier 14, 15 in US20160296563A1; Identifier 31, 32, 33, 34, 35, |
| | 36, 37, 38, 39, 40, 41, 42 in WO2015157252; Identifier 14, 15 in |
| | WO2016139487; Identifier 53, 54, 55, 56, 57, 58 in |
| | US20160250258A1; Identifier 1, 2, 3, 4, 5, 6, 7, 8, 9, 10, 11, 12, 13 |
| | in WO2015187528; Identifier 31, 32, 33, 34, 35, 36, 37, 38, 39, 40, |
| | 41, 42, 58 in WO2015157252; Identifier 31, 32, 33, 34, 35, 36, 37, |
| | 38, 39, 40, 41, 42 in WO2014153270; WO2016134284 (no |
| | Identifier); Identifier 13 in WO2016139487 |
| CD19 or CD33 | Identifier 1 in WO2015075468 which recognizes CD19 OR CD33 |
| CD19/CD20 bispecific | Identifier 1308 in WO2016164731A2; Identifier 2, 8, 11 in |
| | U.S. Pat. No. 9,447.194B2 |
| CD19/IL13 bispecific | Identifier 10 in US20160340649A1 |
| CD2 | Identifier 10, 11 in WO2016138491 |
| CD20 | Identifier 25 in WO2015157399A9; Identifier 177, 181, 182, 183, |
| | 184, 185, 186, 187, 205, 206, 207, 208, 209, 210, 211, 188, 189, 190, |
| | 191, 192, 193, 176, 212, 194, 195, 196, 197, 198, 199, 200, 201, 202, |
| | 203, 178, 179, 180 in WO2016097231 |
| CD22 | Identifier 380, 204, 260, 266, 272, 278, 284, 290, 296, 302, 308, 341, |
| | 213, 320, 326, 332, 338, 347, 350, 356, 362, 368, 374, 219, 386, 392, |
| | 398, 404, 410, 416, 421, 427, 225, 230, 1109, 236, 242, 248, 254 in |
| | WO2016164731A2; Identifier 15, 16, 17, 18, 19, 20, 32 in |
| | WO2013059593; Identifier 22, 23, 24 in US20150299317 |
| CD22/CD19 bispecific | Identifier 29, 30 in WO2016149578; Identifier 1304 in |
| | WO2016164731A2 |
| CD276 | Identifier 39, 40, 41, 42, 43, 44, 45, 46, 47, 122, 123, 124, 125, 126, |
| | 127, 128, 129, 130 in US20160053017 |
| CD3 | Identifier 12 in WO2016138491 |
| CD30 | Identifier 20 in WO2016008973A1; Identifier 1 in |
| | WO2016116035A1; WO2016134284 (no Identifier); Identifier 2 in |
| | WO2016008973 |
| CD33 | Identifier 48, 49, 50, 51, 52, 53, 54, 55, 83 in WO2016014576; |
| | Identifier 19, 20, 21, 22, 23, 24, 25, 26, 27, 28, 29, 30, 31, 32, 33, 34, |
| | 35, 36, 37, 38, 39, 40, 41, 42, 48, 49, 50, 51, 52, 53, 54, 55, 56, 57, |
| | 58, 59, 60, 61, 62, 63, 64, 65, 66, 67, 68, 69, 70, 71 in |
| | WO2015150526A2 |
| CD38 | Identifier 70, 71, 72, 64, 65, 66, 67, 68, 69 in WO2016097231; |
| | Identifier 35, 36, 37 in WO2015121454 |
| CD4 | Identifier 13, 14 in WO2016138491 |
| CD410 | Identifier 7 in EP3074419A2 |
| CD435 | Identifier 5 in EP3074419A2 |
| CD44 | Identifier 21, 22, 23, 24, 25, 26, 27, 28, 31, 32, 33, 34, 35 in |
| | WO2016042461A1 |
| CD4-DDY3 | Identifier 9 in EP3074419A2 |
| CD5 | Identifier 15, 13 in WO2016138491 |
| CD52 | Identifier 18 in WO2016138491 |
| CD7 | Identifier 17 in WO2016138491 |
| CD70 | Identifier 99 in WO2015121454 |
| CD70 | Identifier 100, 93, 94, 96, 101, 95, 97, 98 in WO2015121454 |
| CD8 stalk APRIL | Identifier 51 in US20160296562A1 |
| CEA | Identifier 4 in WO2016008973A1; Identifier 29, 30 in |
| | US20140242701A |
| CLDN6 | Identifier 22, 23, 24 in WO2016150400 |
| CLL1 | Identifier 148 in WO2016179319A1; Identifier 35, 36, 37, 38, 39, 40, |
| | 41, 42, 43, 44, 45, 46, 47, 48, 49, 50, 51, 52, 53, 54, 55, 56, 57, 58, |
| | 59, 60, 61, 62, 63, 64, 65, 66, 67, 68, 69, 70, 71, 72, 73, 74, 75, 76, |
| | 77, 78, 79, 80, 81, 82, 83, 84, 85, 86, 87, 88, 89, 90, 91, 92, 93, 94, |
| | 95, 96, 97, 98, 99, 100, 101, 102, 103, 104, 105, 106, 107, 108, 109, |
| | 110, 111, 112 in WO2016120218; Identifier 91, 92, 93, 94, 95, 96, |
| | 97, 98, 99, 100, 101, 102, 103, 197 in WO2016014535 |
| COM22 | Identifier 358, 359, 360 in US20160297884A1 |
| CS1 | Identifier 55, 57, 60, 54, 56, 48, 49, 50, 51, 52, 53, 58, 59, 61, 62 in |
| | WO2015121454; Identifier 28 in WO2014179759A1 |
| DDD1/AD1 zip | Identifier 37 in WO2016124930 |
| DDD1/AD1-based zip | Identifier 36 in WO2016124930 |
| EGFR | Identifier 3, 2in WO2014130657; Identifier 36, 37, 38, 39, 35 in |
| | US20140242701 A; Identifier 43, 96, 49, 55, 61, 67, 73, 79, 85, 90, 1 |
| | in WO2014130657 |
| EGFR vIII | Identifier 15, 16, 17, 18, 24, 25, 26, 27 in WO2016016341; Identifier |
| | 5, 10, 12, 8, 31, 30, 3 in US20160311907A1; Identifier 10 in |
| | US20160200819A1; Identifier 43, 49, 55, 61, 67, 73, 79, 85, 90, 96 |
| | in U.S. Pat. No. 9,394,368B2; Identifier 49, 55, 61, 67, 73, 79, 85, 90, 2, 1 in |
| | US20170008963A1; Identifier 10, 11 in US20140037628 |
| FcRL5 | Identifier 11 in US20170008963A1 |
| Folate receptor | Identifier 12 in US20170008963A1 |
| FR | Identifier 22 in US20170002072A1 |
| FR beta | Identifier 13, 22 in U.S. Pat. No. 9,402,865B2; Identifier 2, 4, 6 in U.S. Pat. No. 9,446,105B2 |
| FRa | Identifier 13, 14 in US20120213783 |
| Fra | Identifier 959 in WO2016090337; Identifier 13 in |
| | US20170002072A1 |
| GCN4 | Identifier 8, 10 in U.S. Pat. No. 9,446,105B2 |
| GD2 | Identifier 12 in U.S. Pat. No. 9,446,105B2; Identifier 273, 274 in |
| | WO2016168773A3; Identifier 26, 27, 28, 29, 30, 31, 32, 33, 34, 35, |
| | 36, 37 in WO2015132604; WO2016134284 (no Identifier) |
| GD3 | Identifier 19 in WO2016185035A1; Identifier 20, 21, 22, 23, 24, 25, |
| | 26 in WO2016185035A1; WO2016134284 (no Identifier) |
| GFR alpha | Identifier 27 in WO2016185035A1 |
| GPC3 | Identifier 28, 29 in WO2016185035A1; Identifier 3, 27, 10, 29, 14, |
| | 30, 31, 18, 33 in WO2016049459; Identifier 22 in |
| | US20160215261A1 |
| HER2 | Identifier 25 in US20160215261A1; Identifier 9, 10 of |
| | WO2016073629; Identifier 17, 28, 98, 110 in US20160333114A1; |
| | Identifier 271, 272 in WO2016168773A3; Identifier 5 in |
| | WO2016168769A1 |
| Herl/Her3 bispecific | Identifier 23, 24 in US20160215261A1 |
| HERVK | Identifier 6 in WO2016168769A1 |
| HIV Env | Identifier 48, 49 in WO2016168766A1; Identifier 4 in |
| | EP2997134A4; Identifier 7, 9, 47, 49 in WO2015077789 |
| HSP70 | Identifier 51, 53, 5 in WO2015077789; Identifier 21, 22, 23, 24, 25, |
| | 26, 27, 28, 29 in WO2016120217 |
| IL13 | Identifier 30, 31, 32 in WO2016120217 |
| IL13Ra2specific | Identifier 4, 5, 6 in WO2016089916A1; Identifier 47, 49 in |
| | WO2016123143; Identifier 51, 53, 55 in WO2016123143; Identifier |
| | 1, 31, 32, 33, 34, 35, 36, 37, 38, 39, 40, 41, 42, 43, 44, 45 in |
| | US20160340649A1 |
| KMA | Identifier 46 in US20160340649A1 |
| Mesothelin | Identifier 47, 48 in US20160340649A1; Identifier 48 in |
| | US20160340649A1; Identifier 27 in WO2016172703A2; Identifier |
| | 18, 19, 20, 21, 22, 23 in WO2013142034; Identifier 3 in |
| | WO2013067492 |
| MUC1 | Identifier 5, 7 in WO2013063419; Identifier 51 in |
| | US20160340406A1; Identifier 30, 32, 34 in US20160130357; |
| | Identifier 295, 298, 301, 304, 307, 607, 609, 611, 613 in |
| | WO2016130726 |
| NCAR with RQR82 | Identifier 615 in WO2016130726 |
| ACD 19 | |
| NYBR1 | Identifier 617, 619 in WO2016130726; Identifier 218 in |
| | WO2016097231; Identifier 26, 29, 60 in WO2015112830; Identifier |
| | 1 in US20160333422A1 |
| P5A | Identifier 26, 29, 60 in US20160333422A1 |
| P5AC1 | Identifier 343, 344, 345, 346 in US20160297884A1 |
| P5AC16 | Identifier 347, 396, 348 in US20160297884A1 |
| P6AP | Identifier 349, 350, 351 in US20160297884A1 |
| P6DY | Identifier 364, 365, 366 in US20160297884A1 |
| PC1C12 | Identifier 352, 353, 354 in US20160297884A1 |
| PCI | Identifier 361, 362, 363 in US20160297884A1 |
| PD1 | Identifier 355, 356, 357 in US20160297884A1; Identifier 119 in |
| | WO2014153270; Identifier 121 in WO2014153270; Identifier 22, 24, |
| | 63, 64, 65, 66, 67, 68, 69, 70, 71, 72, 73, 74, 75, 76, 77, 78, 79, 80, |
| | 81, 82, 83, 84, 85, 86 in US20160311917A1; Identifier 26, 39 in |
| | WO2016172537A1; Identifier 40 in US20160311907A1; Identifier |
| | 121, 119 in WO2015157252; Identifier 24 in WO2016014565; |
| | Identifier 22 in WO2016014565 |
| PD1 | Identifier 23 in WO2016014565; Identifier 26 in WO2015142675 |
| PSMA | Identifier 39 in WO2015142675; Identifier 28, 29 in |
| | US20160311907A1; Identifier 140, 144, 145, 146, 147, 148, 149, |
| | 150, 167, 168, 169, 170, 171, 172, 173, 174, 151, 152, 153, 154, 155, |
| | 156, 139, 138, 175, 157, 158, 159, 160, 161, 162, 163, 164, 165, 166, |
| | 141, 142, 143, 214 in WO2016097231 |
| ROR1 | Identifier 216, 217, 215 in WO2016097231; Identifier 79, 80, 81, 82, |
| | 83, 84, 85, 86, 87, 88, 89, 90, 91, 92, 93, 94, 95, 96, 103, 104, 105, |
| | 106, 107, 108, 109, 110, 111, 112, 113, 114, 115, 116, 117, 118, 119, |
| | 120, 127, 128, 129, 130, 131,132, 133,134, 1335, 136, 137, 138, 97, |
| | 98, 99, 100, 101, 102, 121, 122, 123, 124, 125, 126 in |
| | WO2016016344A1; Identifier 386, 387, 388, 389, 390, 391, 392, |
| | 393, 394 in WO2016187216A1 |
| SNAP | Identifier 395 in WO2016187216A1 |
| SSEA4 | Identifier 396, 397 in WO2016187216A1 |
| Tan | Identifier 19 in US20160311907A1 recognizes CD19 AND CD33 |
| | using a CD45 phosphatase; Identifier 5 in WO2016026742A1 |
| | recognizes CD19 AND CD33 using a CD148 phosphatase; Identifier |
| | 6 in WO2016026742A1 which recognizes CD19 AND NOT CD33 |
| | and is based on an ITIM containing endodomain from LAIR1; |
| | Identifier 3 in WO2015075468 which recognizes CD19 AND NOT |
| | CD33 based on PTPN6 phosphatase; Identifier 2 in WO2015075468 |
| | which recognizes CD19 AND NOT CD33 and recruits a |
| | PTPN6/CD148 fusion protein to an ITIM containing endodomain |
| TOSO | Identifier 5, 4 in WO2015075468 |
| Trophoblast | Identifier 6 in WO2015075468; Identifier 4 in US20160347854A1; |
| Glycoprotein 5T4 | Identifier 4 in EP3098237A1; Identifier 19, 20, 21, 22, 23, 24, 25, 26, |
| | 27, 28, 29, 30, 31, 32, 33, 34, 35, 36, 37, 38, 39 in |
| | WO2016034666A1 |
| TSLPR | Identifier 40, 41, 42 in WO2016034666A1; Identifier 39, 40, 41, 42, |
| | 43, 44, 45, 46 in WO2015084513; Identifier 39, 40, 41, 42, 43 in |
| | US20160311910A1 |
| VEGFR2 | Identifier 44, 45, 46 in US20160311910A1; Identifier 10, 11, 12 in |
| | US20120213783 |
| VNAR | Identifier 105, 106, 107, 108, 109, 110 in US20160333094A1 |

ii. Linkers

In some embodiments, an scFv fragment of an extracellular domain of a CAR includes a linker between the VH and VL domains. The linker can be a peptide linker and may include any naturally occurring amino acid. Exemplary amino acids that may be included into the linker are Gly, Ser Pro, Thr, Glu, Lys, Arg, Ile, Leu, His and Phe. The linker should have a length that is adequate to connect the VH and the VL in such a way that they form the correct conformation relative to one another so that they retain the desired activity, such as binding to an antigen. The linker may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. Exemplary linkers that may be used are Gly rich linkers, Gly and Ser containing linkers, Gly and Ala containing linkers, Ala and Ser containing linkers, and other flexible linkers.

In some embodiments, the linker is a Whitlow linker. In one embodiment, the Whitlow linker includes the amino acid sequence set forth in SEQ ID NO:3, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:3 of PCT/US2021/072646.

In another embodiment, the linker is a (G4S)₃ linker. In one embodiment, the (G4S)₃ linker includes the amino acid sequence set forth in SEQ ID NO: 25, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:25 of PCT/US2021/072646.

Other linker sequences may include portions of immunoglobulin hinge area, CL or CH1 derived from any immunoglobulin heavy or light chain isotype. Exemplary linkers that may be used include any of SEQ ID NOs:26-56 in Table 1 of PCT/US2021/072646, the disclosure including the sequence listing is incorporated herein by reference. Additional linkers are described for example in WO2019/060695, incorporated by reference herein in its entirety.

The linkers described herein including SEQ ID NOS: 40-73 of Table 4 can be used in any of the polypeptides provided including those containing CD16, NKG2D, IL-15, IL-15Rα, HLA-E, HLA-G, HSV-TK, PSMA, and the like.

Table 4 provides exemplary linkers (SEQ ID NOS:40-73), which correspond to SEQ ID NOS: 3 and 25-56 of U.S. application Ser. No. 17/657,803 filed Apr. 4, 2022, the contents of which are incorporated herein by reference in its entirety.

TABLE 4

| | | SEQ ID | SEQ ID NO: of |
|---|---|---|---|
| Name | Linker Sequence | NO: | US17/657,803 |
| Whitlow | GSTSGSGKPGSGEGSTKG | 40 | 3 |
| Linker | | | |
| GGGGS or | GGGGS | 41 | |
| multimer | | | |
| thereof | | | |
| (G₄S)₃ | GGGGSGGGGSGGGGS | 42 | 25 |
| Linker 3 | GGSEGKSSGSGSESKSTGGS | 43 | 26 |
| Linker 4 | GGGSGGGS | 44 | 27 |
| Linker 5 | GGGSGGGSGGGS | 45 | 28 |
| Linker 6 | GGGSGGGSGGGSGGGS | 46 | 29 |
| Linker 7 | GGGSGGGSGGGSGGGSGGGS | 47 | 30 |
| Linker 8 | GGGGSGGGGSGGGGSGGGGS | 48 | 31 |
| Linker 9 | GGGGSGGGGSGGGGSGGGGSGGGGS | 49 | 32 |
| Linker 10 | IRPRAIGGSKPRVA | 50 | 33 |
| Linker 11 | GKGGSGKGGSGKGGS | 51 | 34 |
| Linker 12 | GGKGSGGKGSGGKGS | 52 | 35 |
| Linker 13 | GGGKSGGGKSGGGKS | 53 | 36 |
| Linker 14 | GKGKSGKGKSGKGKS | 54 | 37 |
| Linker 15 | GGGKSGGKGSGKGGS | 55 | 38 |
| Linker 16 | GKPGSGKPGSGKPGS | 56 | 39 |
| Linker 17 | GKPGSGKPGSGKPGSGKPGS | 57 | 40 |
| Linker 18 | GKGKSGKGKSGKGKSGKGKS | 58 | 41 |
| Linker 19 | STAGDTHLGGEDFD | 59 | 42 |
| Linker 20 | GEGGSGEGGSGEGGS | 60 | 43 |
| Linker 21 | GGEGSGGEGSGGEGS | 61 | 44 |
| Linker 22 | GEGESGEGESGEGES | 62 | 45 |
| Linker 23 | GGGESGGEGSGEGGS | 63 | 46 |
| Linker 24 | GEGESGEGESGEGESGEGES | 64 | 47 |
| Linker 25 | GSTSGSGKPGSGEGSTKG | 65 | 48 |
| Linker 26 | PRGASKSGSASQTGSAPGS | 66 | 49 |
| Linker 27 | GTAAAGAGAAGGAAAGAAG | 67 | 50 |
| Linker 28 | GTSGSSGSGSGGSGSGGGG | 68 | 51 |
| Linker 29 | GKPGSGKPGSGKPGSGKPGS | 69 | 52 |
| Linker 30 | GSGS | 70 | 53 |
| Linker 31 | APAPAPAPAP | 71 | 54 |
| Linker 32 | APAPAPAPAPAPAPAPAPAP | 72 | 55 |
| Linker 33 | AEAAAKEAAAKEAAAAKEAAAAKEAAAAK | 73 | 56 |
| | AAA | | |

### B. Signal Peptides

In some embodiments, a CAR polypeptide includes a signal peptide (e.g., a leader peptide or localization peptide). The signal peptide may be positioned at the N-terminus of the extracellular domain. The signal peptide may be optionally cleaved from the extracellular domain during cellular processing and localization of the CAR to the cellular membrane. Any of various signal peptide sequences known to one of skill in the art may be used. Non-limiting examples of signal peptides from which the sequence may be derived include granulocyte-macrophage colony-stimulating factor receptor (GMCSFR), FcεR, human immunoglobulin (IgG) heavy chain (HC) variable region, CD8a, or any of various other proteins secreted by T cells. In some embodiments, the signal sequence is compatible with the secretory pathway of a T cell. In certain embodiments, the signal sequence is derived from a human immunoglobulin heavy chain.

In some embodiments, the signal sequence is derived from GMCSFR. In one embodiment, the GMCSFR signal sequence includes the amino acid sequence set forth in SEQ ID NO: 1, or a variant thereof having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity to SEQ ID NO:1 as set forth in PCT/US2021/072646, the disclosure of which is incorporated herein by reference.

### C. Transmembrane Domains

In some embodiments, a CAR polypeptide includes a transmembrane domain, fused in frame between an extracellular domain and a cytoplasmic domain.

The transmembrane domain may be derived from the protein contributing to the extracellular domain, the protein contributing the signaling or co-signaling domain, or by a completely different protein. In some embodiments, the transmembrane domain is selected or modified by amino acid substitution, deletions, or insertions to minimize interactions with other members of the CAR polypeptide. In some instances, the transmembrane domain is selected or modified by amino acid substitution, deletions, or insertions to avoid binding of proteins naturally associated with the transmembrane domain. In some embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains may be derived from (i.e. comprise at least the transmembrane region(s) of) the α, β or ζ chain of the T-cell receptor (TCR), CD28, CD3 epsilon, CD45, CD4, CD5, CD8, CD8a, CD9, CD16, CD22, CD33, CD37, CD40, CD64, CD80, CD86, CD134, CD137, or CD154. In some instances, the transmembrane domain may be synthetic, in which case it may include predominantly hydrophobic residues such as leucine and valine. For example, a triplet of phenylalanine, tryptophan and/or valine can be found at each end of a synthetic transmembrane domain.

In some embodiments, it is desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some embodiments, the transmembrane domain is selected or modified by amino acid substitution to avoid binding of such domains to the transmembrane domains of the same or different surface membrane proteins to minimize interactions with other members of the receptor complex. In some embodiments, it is desirable to employ the transmembrane domain of ζ, η or FcεR1γ and −β, MB1 (Igα), B29 or CD3-γ, ζ, or η, in order to retain physical association with other members of the receptor complex.

In some embodiments, the transmembrane domain of a CAR is derived from CD8 or CD28. In some embodiments, the CD8 transmembrane domain comprises the amino acid sequence set forth in SEQ ID NO:23, or a variant thereof having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity to SEQ ID NO:23 as set forth in PCT/US2021/072646, the disclosure of which is incorporated herein by reference. In an embodiment, the CD28 transmembrane domain comprises the amino acid sequence set forth in SEQ ID NO:24, or a variant thereof having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity to SEQ ID NO:24 as set forth in PCT/US2021/072646, the disclosure of which is incorporated herein by reference.

### D. Hinge Regions

In some embodiments, a CAR polypeptide includes a hinge region (e.g., a spacer region) between an extracellular domain and a transmembrane domain, such that the extracellular domain, hinge region, and transmembrane domain are in frame with each other.

A hinge region may contain up to 300 amino acids, preferably 10 to 100 amino acids and most preferably 25 to 50 amino acids. A hinge region may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring spacer region sequence, or may be an entirely synthetic spacer region sequence. Non-limiting examples of hinge regions include a part of human CD8a chain, partial extracellular domain of CD28, FcγRlIIIa receptor, IgG, IgM, IgA, IgD, IgE, an Ig hinge, or functional fragment thereof. In some embodiments, additional linking amino acids are added to the hinge region to ensure that the antigen-binding domain is an optimal distance from the transmembrane domain. In some embodiments, when the hinge region is derived from an immunoglobulin, the region may be mutated to prevent Fc receptor binding.

In some embodiments, the hinge region includes a hinge domain of a recognized protein. The hinge domain may be derived from CD8a, CD28, or an immunoglobulin (IgG). For example, the IgG hinge may be from IgG1, IgG2, IgG3, IgG4, IgM1, IgM2, IgA1, IgA2, IgD, IgE, or a chimera thereof.

In some embodiments, the hinge domain comprises an immunoglobulin IgG hinge or functional fragment thereof. In certain embodiments, the IgG hinge is from IgG1, IgG2, IgG3, IgG4, IgM1, IgM2, IgA1, IgA2, IgD, IgE, or a chimera thereof. In various embodiments, the hinge domain comprises the CH1, CH2, CH3 and/or hinge region of the immunoglobulin. In many embodiments, the hinge domain comprises the core hinge region of the immunoglobulin. The term “core hinge” can be used interchangeably with the term “short hinge” (“SH”). Non-limiting examples of suitable hinge domains are the core immunoglobulin hinge regions include EPKSCDKTHTCPPCP (SEQ ID NO:74; SEQ ID NO:57 of PCT/US2021/072646) from IgG1, ERKCCVECPPCP (SEQ ID NO:75; SEQ ID NO: 58 of PCT/US2021/072646) from IgG2, ELKTPLGDTTHTCPRCP(EPKSCDTPPPCPRCP)₃ (SEQ ID NO:76; SEQ ID NO: 59 of PCT/US2021/072646) from IgG3, and ESKYGPPCPSCP (SEQ ID NO:77; SEQ ID NO: 60 of PCT/US2021/072646) from IgG4 (see also Wypych et al., JBC 2008 283(23): 16194-16205, which is incorporated herein by reference in its entirety for all purposes). In many embodiments, the hinge domain is a fragment of the immunoglobulin hinge.

In some embodiments, the hinge domain is derived from CD8 or CD28. In one embodiment, the CD8 hinge domain comprises the amino acid sequence set forth in SEQ ID NO:21, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:21 of PCT/US2021/072646, the disclosure of which is incorporated herein by reference.

In one embodiment, the CD28 hinge domain comprises the amino acid sequence set forth in SEQ ID NO:22, or a variant thereof having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99%, sequence identity to SEQ ID NO:22 of PCT/US2021/072646, the disclosure of which is incorporated herein by reference.

In some embodiments, the transmembrane domain and/or hinge domain is derived from CD8 or CD28. In some embodiments, both the transmembrane domain and hinge domain are derived from CD8. In some embodiments, both the transmembrane domain and hinge domain are derived from CD28.

### E. Cytoplasmic Domains Including Co-Stimulatory Domains

In some aspects, a CAR polypeptide includes a cytoplasmic domain, which contains at least one intracellular signaling domain. In some embodiments, a cytoplasmic domain also comprises one or more co-stimulatory signaling domains.

The cytoplasmic domain is responsible for activation of at least one of the normal effector functions (e.g., specialized function) of the host cell (e.g., T cell) in which the CAR has been placed in. The term “effector function” refers to a specialized function of a cell. Effector function of a T-cell, for example, may be cytolytic activity or helper activity including the secretion of cytokines. A signaling domain can include a portion of a protein which transduces the effector function signal and directs the cell to perform a specialized function. While usually the entire signaling domain is present, in many cases it is not necessary to use the entire domain. To the extent that a truncated portion of the intracellular signaling domain is used, such truncated portion may be used in place of the intact chain as long as it transduces the effector function signal. An intracellular signaling domain includes any truncated portion of the signaling domain sufficient to transduce the effector function signal. Non-limiting examples of signaling domains which can be used include, e.g., signaling domains derived from DAP10, DAP12, Fc epsilon receptor I γ chain (FCER1G), FcRβ, CD36, CD3R, CD37, CD3ζ, CD5, CD22, CD226, CD66d, CD79A, and CD79B.

In some embodiments, the cytoplasmic domain comprises a CD3ζ signaling domain. In some embodiments, the CD3ζ signaling domain includes the amino acid sequence set forth in SEQ ID NO:6, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:6 of PCT/US2021/072646, the disclosure of which is incorporated herein by reference in its entirety.

In some embodiments, the cytoplasmic domain contains one or more co-stimulatory signaling domains. In some embodiments, the one or more co-stimulatory signaling domains are derived from CD28, 41BB, IL2Rb, CD40, OX40 (CD134), CD80, CD86, CD27, ICOS, NKG2D, DAP10, DAP12, 2B4 (CD244), BTLA, CD30, GITR, CD226, CD79A, and HVEM in its entirety.

In some embodiments, the co-stimulatory signaling domain is derived from 4-1BB. In one embodiment, the 4-1BB co-stimulatory signaling domain comprises the amino acid sequence set forth in SEQ ID NO:8, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:8 of PCT/US2021/072646, the disclosure of which is incorporated herein by reference in its entirety.

In some embodiments, the co-stimulatory signaling domain is derived from IL2Rb. In one embodiment, the IL2Rb co-stimulatory signaling domain comprises the amino acid sequence set forth in SEQ ID NO:9, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:9 of PCT/US2021/072646, the disclosure of which is incorporated herein by reference in its entirety.

In some embodiments, the co-stimulatory signaling domain is derived from CD40. In one embodiment, the CD40 co-stimulatory signaling domain includes the amino acid sequence set forth in SEQ ID NO:10, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:10 of PCT/US2021/072646, the disclosure of which is incorporated herein by reference in its entirety.

In some embodiments, the co-stimulatory signaling domain is derived from OX40. In one embodiment, the OX40 co-stimulatory signaling domain includes the amino acid sequence set forth in SEQ ID NO:11, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:11 of PCT/US2021/072646, the disclosure of which is incorporated herein by reference in its entirety.

In some embodiments, the co-stimulatory signaling domain is derived from CD80. In one embodiment, the CD80 co-stimulatory signaling domain includes the amino acid sequence set forth in SEQ ID NO:12, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:12 of PCT/US2021/072646, the disclosure of which is incorporated herein by reference in its entirety.

In some embodiments, the co-stimulatory signaling domain is derived from CD86. In one embodiment, the CD86 co-stimulatory signaling domain includes the amino acid sequence set forth in SEQ ID NO: 13, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:13 of PCT/US2021/072646, the disclosure of which is incorporated herein by reference in its entirety.

In some embodiments, the co-stimulatory signaling domain is derived from CD27. In one embodiment, the CD27 co-stimulatory signaling domain includes the amino acid sequence set forth in SEQ ID NO:14, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:14 of PCT/US2021/072646, the disclosure of which is incorporated herein by reference in its entirety.

In some embodiments, the co-stimulatory signaling domain is derived from ICOS. In one embodiment, the ICOS co-stimulatory signaling domain includes the amino acid sequence set forth in SEQ ID NO:15, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:15 of PCT/US2021/072646, the disclosure of which is incorporated herein by reference in its entirety.

In some embodiments, the co-stimulatory signaling domain is derived from NKG2D. In one embodiment, the NKG2D co-stimulatory signaling domain includes the amino acid sequence set forth in SEQ ID NO:16, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:16 of PCT/US2021/072646, the disclosure of which is incorporated herein by reference in its entirety.

In some embodiments, the co-stimulatory signaling domain is derived from DAP10. In one embodiment, the DAP10 co-stimulatory signaling domain includes the amino acid sequence set forth in SEQ ID NO: 17, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:17 of PCT/US2021/072646, the disclosure of which is incorporated herein by reference in its entirety.

In some embodiments, the co-stimulatory signaling domain is derived from DAP12. In one embodiment, the DAP12 co-stimulatory signaling domain includes the amino acid sequence set forth in SEQ ID NO:18, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:18 of PCT/US2021/072646, the disclosure of which is incorporated herein by reference in its entirety.

In some embodiments, the co-stimulatory signaling domain is derived from 2B4 (CD244). In one embodiment, the 2B4 (CD244) co-stimulatory signaling domain includes the amino acid sequence set forth in SEQ ID NO:19, or a variant thereof having at least 50%, at least 55%, at least 60%, at least 65%, at least 70%, at least 75%, at least 80%, at least 85%, at least 90%, at least 95%, at least 96%, at least 97%, at least 98% or at least 99%, sequence identity to SEQ ID NO:19 of PCT/US2021/072646, the disclosure of which is incorporated herein by reference in its entirety.

In some embodiments, the CAR polypeptide includes one costimulatory signaling domains. In many embodiments, the CAR includes 2 or more costimulatory signaling domains. In various embodiments, the CAR includes 2, 3, 4, 5, 6, or more costimulatory signaling domains.

### V. Non-Classical HLA Class I

In some embodiments, an iPSC or derivative cell thereof can be further modified by introducing an exogenous polynucleotide encoding one or more proteins related to immune evasion, such as non-classical HLA class I proteins (e.g., HLA-E and HLA-G). In some cases, disruption of the beta-2-microglobulin (B2M) gene eliminates surface expression of all MHC class I molecules, leaving cells vulnerable to lysis by NK cells through the “missing self” response. Exogenous HLA-E expression can lead to resistance to NK-mediated lysis (Gornalusse et al., Nat Biotechnol., 2017 August; 35(8): 765-772). In some embodiments, an iPSC or derivative cell thereof is engineered to exogenously express HLA-E and/or HLA-G. In certain embodiments, an iPSC or derivative cell thereof with disruption or elimination of B2M expression is engineered to exogenously express HLA-E and/or HLA-G.

In some embodiments, the iPSC or derivative cell thereof comprises an exogenous polypeptide encoding at least one of a human leukocyte antigen E (HLA-E) and human leukocyte antigen G (HLA-G). In some embodiments, the iPSC or derivative cell thereof comprises an exogenous polynucleotide encoding HLA-E, HLA-G or both HLA-E and HLA-G. In many embodiments, the exogenous polynucleotide encodes HLA-E and HLA-G such that they are operably linked by an autoprotease peptide. For example, the polynucleotide can include from 5′ to 3′ order: an HLA-E sequence, a P2A sequence, and an HLA-G sequence. In some cases, the polynucleotide can include from 5′ to 3′ order: an HLA-G sequence, a P2A sequence, and an HLA-E sequence.

In some embodiments, the full-length HLA-E protein has the amino acid sequence set forth in NCBI Ref Seq. No. NP_005507.3 or UniProt No. P13747. In some instance, the coding sequence of full-length HLA-E is set forth in NCBI Ref. No. NM_005516.5. In some embodiments, the mature HLA-E protein has the sequence from amino acid positions 22-358 of the sequence set forth in NCBI Ref Seq. No. NP_005507.3 or UniProt No. P13747.

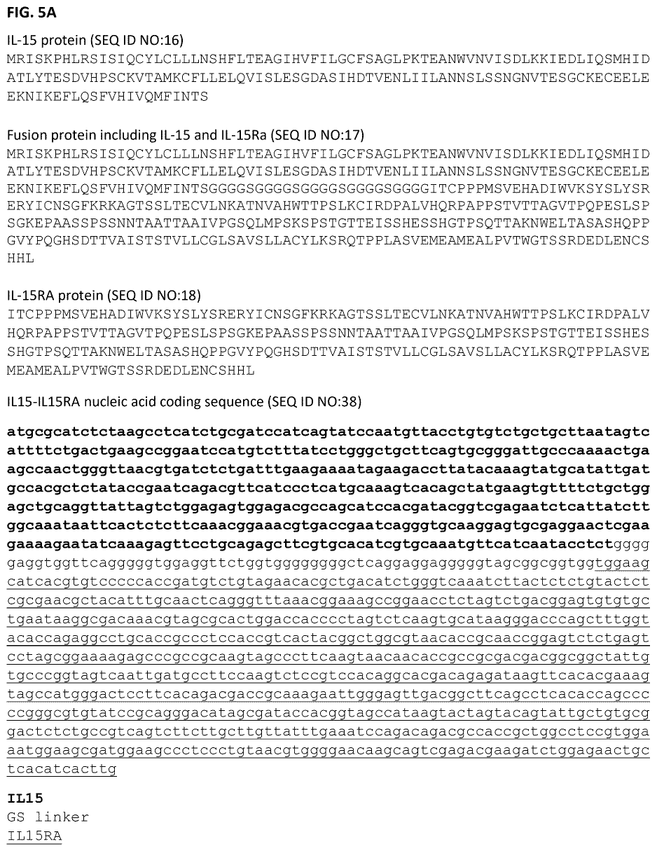

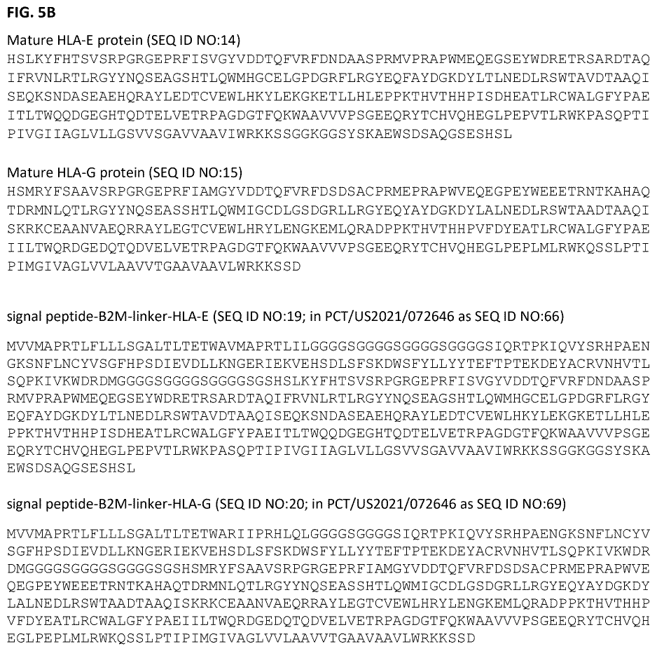

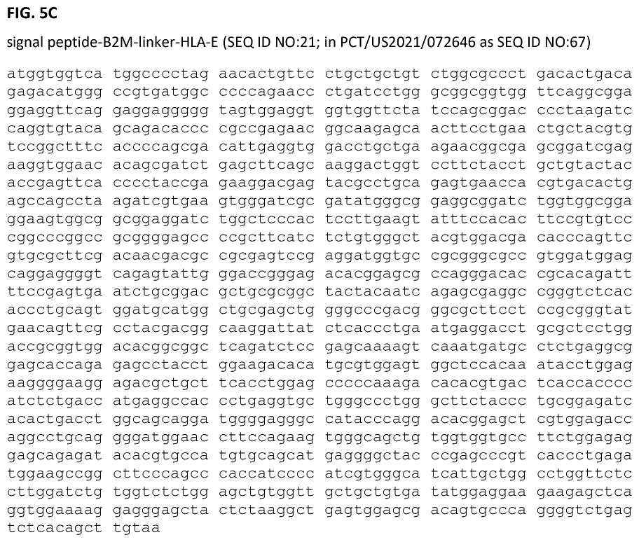

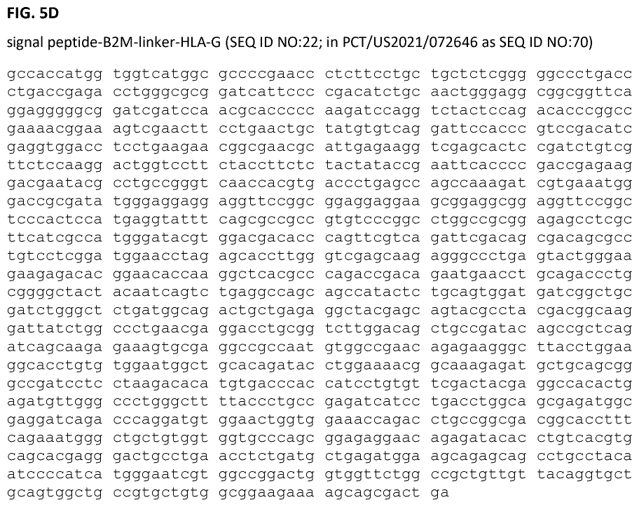

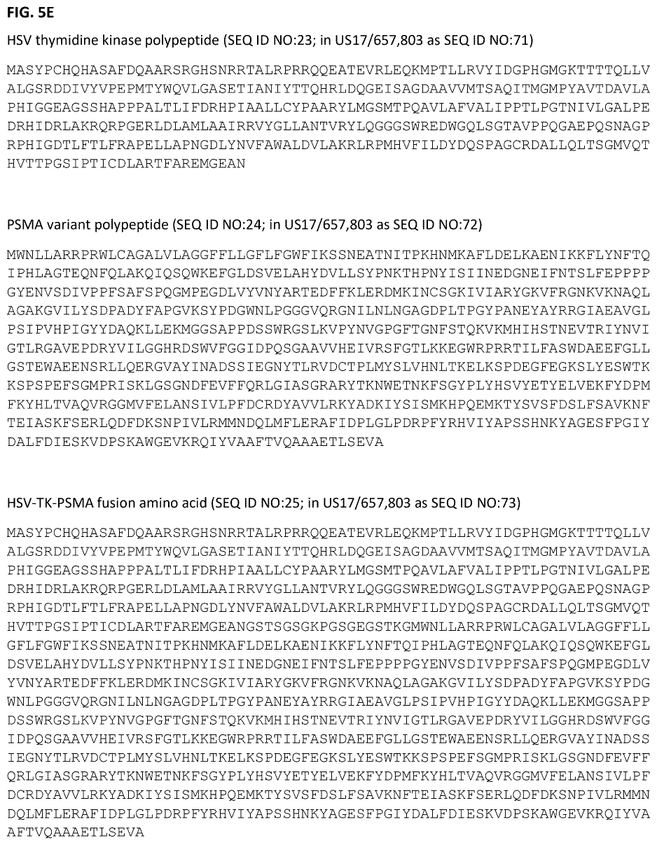

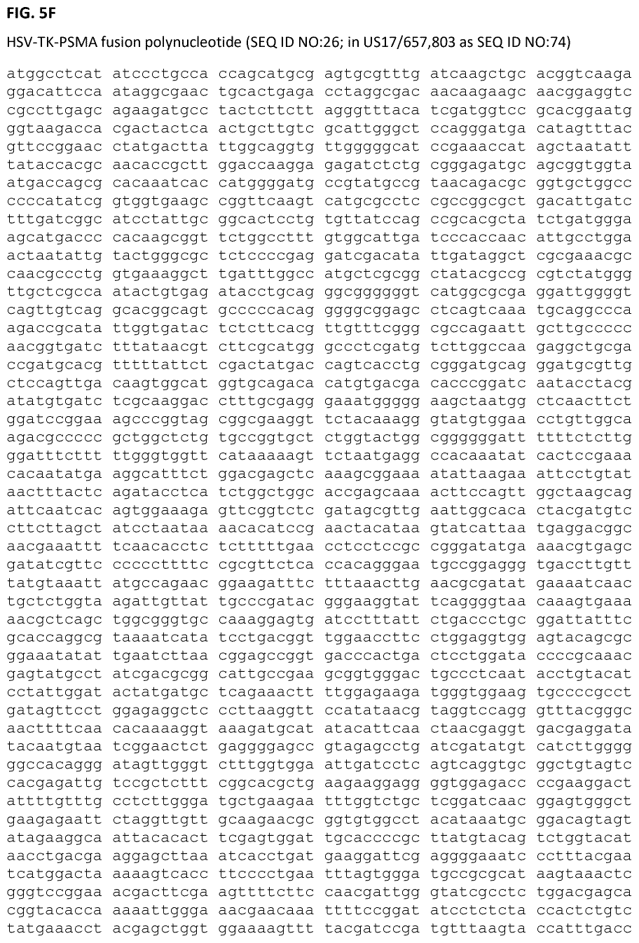

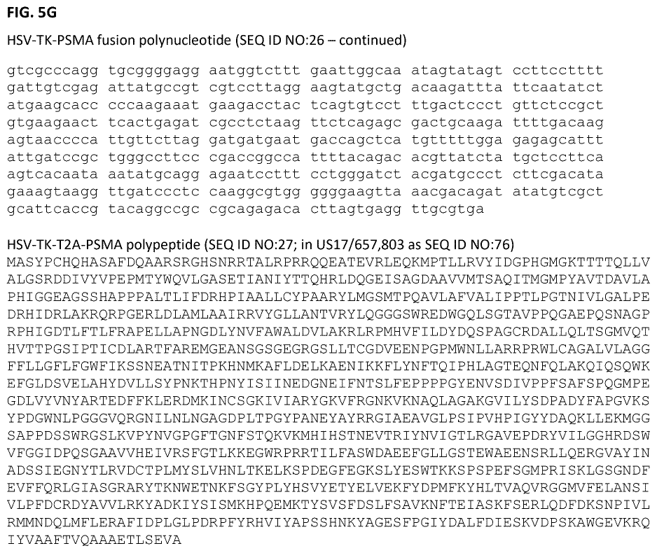

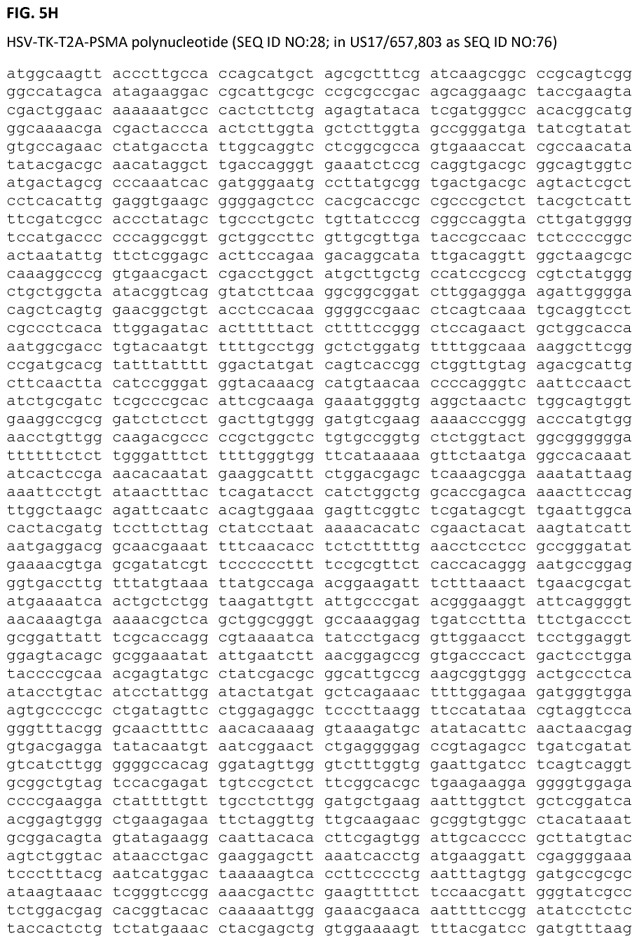

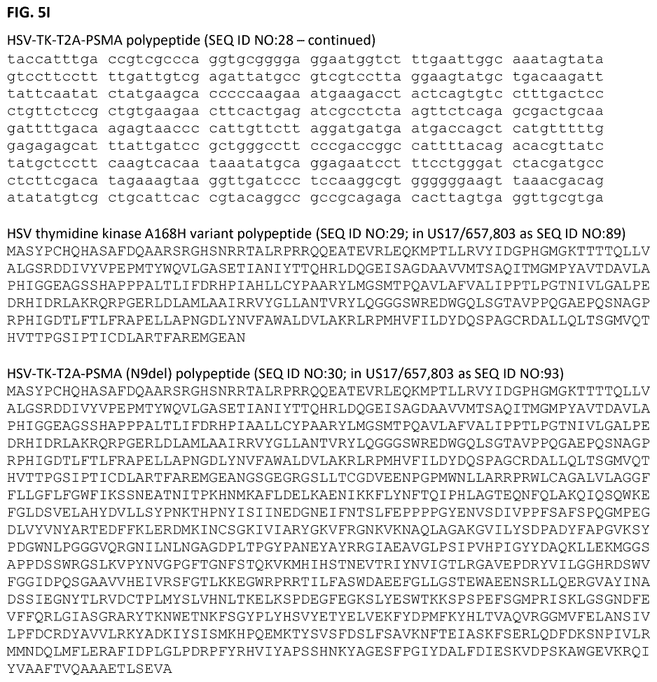

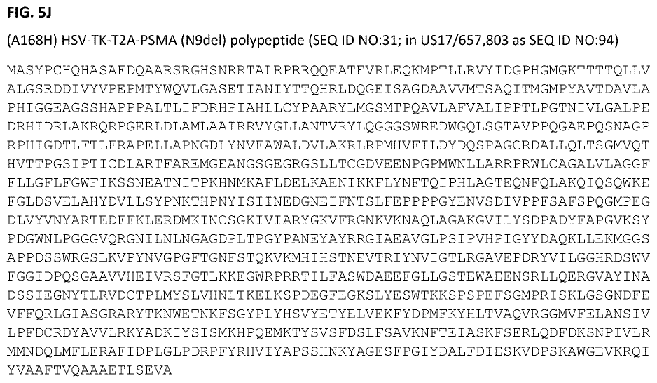

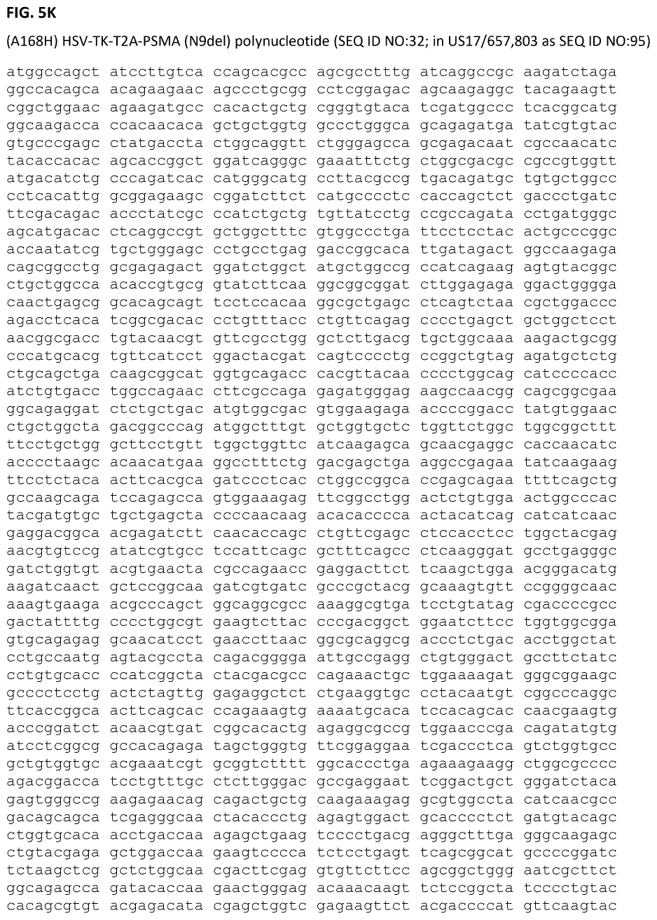

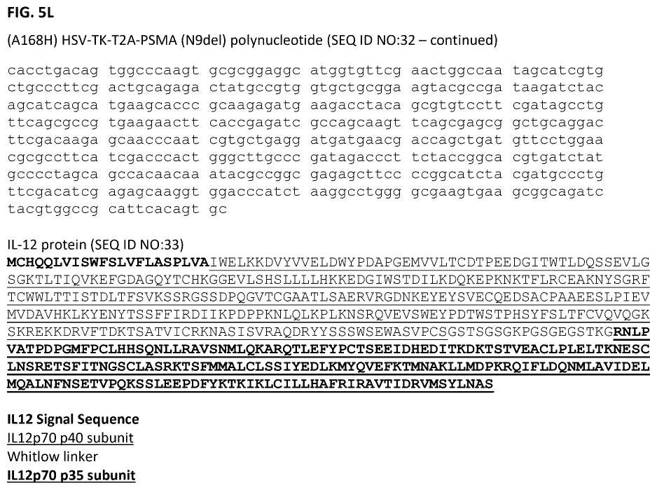

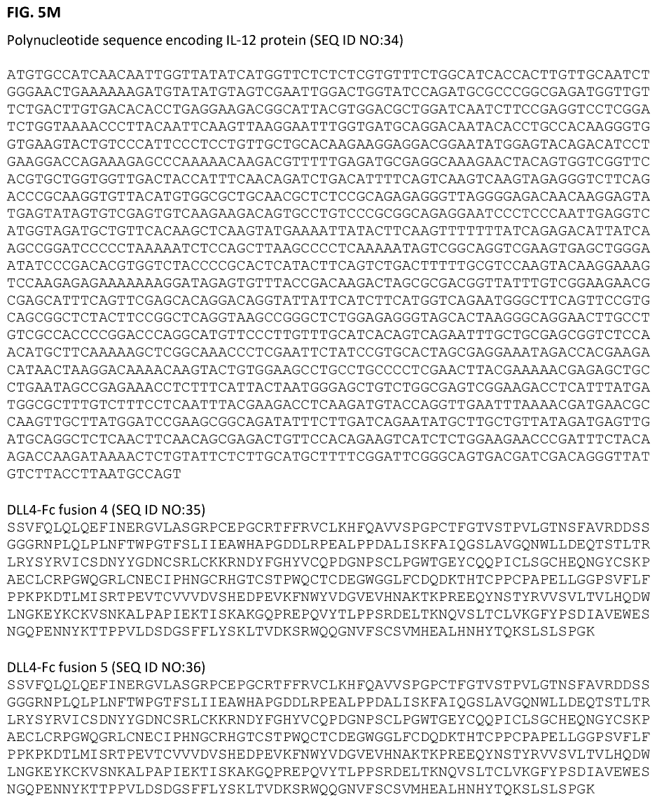

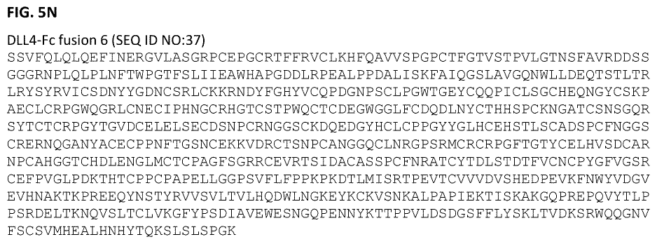

In some embodiments, the HLA-E protein comprises an amino acid sequence having at least 90%, such as at least 90%, 91%, 92%, 93%, 94%, 95%, 96%, 97%, 98%, 99% or 100%, sequence identity to SEQ ID NO:65, preferably the amino acid sequence of SEQ ID NO:65 as set forth in PCT/US2021/072646, the disclosure of which is incorporated herein by reference. In some embodiments, the HLA-E protein comprises an amino acid sequence having at least 90%, such as at least 90%, 91%, 92%, 93%, 94%, 95%, 96%, 97%, 98%, 99% or 100%, sequence identity to SEQ ID NO:14, preferably the amino acid sequence of SEQ ID NO:14 as set forth in FIG. 5.

In some embodiments, the full-length HLA-G protein has the amino acid sequence set forth in NCBI Ref Seq. No. NP_002118.1 or UniProt No. P17693. In some instance, the coding sequence of full-length HLA-G is set forth in NCBI Ref No. NM_002127.5. In some embodiments, the mature HLA-G protein has the sequence from amino acid positions 25-358 of the sequence set forth in NCBI Ref Seq. No. NP_002118.1 or UniProt No. P17693.

In some embodiments, the HLA-G protein comprises an amino acid sequence having at least 90%, such as at least 90%, 91%, 92%, 93%, 94%, 95%, 96%, 97%, 98%, 99% or 100%, sequence identity to SEQ ID NO:68, preferably SEQ ID NO:68 as set forth in PCT/US2021/072646, the disclosure of which is incorporated herein by reference. In some embodiments, the HLA-G protein comprises an amino acid sequence having at least 90%, such as at least 90%, 91%, 92%, 93%, 94%, 95%, 96%, 97%, 98%, 99% or 100%, sequence identity to SEQ ID NO:15, preferably SEQ ID NO:15 as set forth FIG. 5.

In various embodiments, the exogenous polynucleotide encodes a polypeptide comprising a signal peptide operably linked to a mature B2M protein that is fused to an HLA-E via a linker (including those described herein). In some embodiments, the exogenous polypeptide comprises an amino acid sequence having at least 90%, such as at least 90%, 91%, 92%, 93%, 94%, 95%, 96%, 97%, 98%, 99% or 100%, sequence identity to SEQ ID NO:66, preferably SEQ ID NO:66 as set forth in PCT/US2021/072646, the disclosure of which is incorporated herein by reference. In various embodiments, the exogenous polynucleotide encodes a polypeptide comprising a signal peptide operably linked to a mature B2M protein that is fused to an HLA-G via a linker (including those described herein). In some embodiments, the exogenous polypeptide comprises an amino acid sequence having at least 90%, such as at least 90%, 91%, 92%, 93%, 94%, 95%, 96%, 97%, 98%, 99% or 100%, sequence identity to SEQ ID NO:69, preferably SEQ ID NO:69 as set forth in PCT/US2021/072646, the disclosure of which is incorporated herein by reference.

In some instances, the exogenous polynucleotide encodes an HLA polypeptide that includes a signal peptide, such as an HLA-E signal peptide, which is linked to an HLA sequence, such as a mature B2M protein, and/or a mature HLA-E protein. In some embodiments, the B2M and HLA-E are linked by a flexible linker such as, but not limited to, a glycine-serine linker and other described herein. In some instances, the HLA polypeptide that includes a signal peptide, such as an HLA-G signal peptide, which is linked to the HLA sequence, such as a mature B2M protein, and/or a mature HLA-G protein. In some embodiments, the B2M and HLA-G are linked by a flexible linker such as, but not limited to, a glycine-serine linker and other described herein.

### VI. IL-15 and IL-15Ra Polypeptides

In some embodiments, an iPSC or derivative cell thereof is modified to express exogenous polynucleotide encoding an IL-15 protein. In some embodiments, an iPSC or derivative cell thereof is modified to express an exogenous polynucleotide encoding a fusion protein comprising an IL-15 protein and an IL-15 receptor alpha (IL-15Rα, IL-15Rα, and IL-15RA). Such an exogenous polynucleotide construct can be introduced into a specific genomic site or gene locus of the iPSC or derivative cell.

In some embodiments, a full-length IL-15 protein has the amino acid sequence set forth in NCBI Ref. Seq. Nos. NP_000576.1 or NP_751915.1 or UniProt No. P40933. In some instance, the coding sequence of full-length IL-15 is set forth in NCBI Ref. Nos. NM_000585.4 or NM_172175.2. In some embodiments, the mature IL-15 protein has the sequence from amino acid positions 49-162 of the sequence set forth in NCBI Ref. Seq. Nos. NP_000576.1 or NP_751915.1 or UniProt No. P40933. In some embodiments, the IL-15 protein includes an amino acid sequence having at least 90%, such as at least 90%, 91%, 92%, 93%, 94%, 95%, 96%, 97%, 98%, 99% or 100%, sequence identity to SEQ ID NO:16, preferably the amino acid sequence of SEQ ID NO:16 as set forth in FIG. 5.

In some embodiments, a full-length IL-15Ra protein has the amino acid sequence set forth in NCBI Ref. Seq. Nos. NP_001230468.1, NP_001243694.1. NP_002180.1 or NP_751950.2 or UniProt No. Q13261. In some instance, the coding sequence of full-length IL-15Ra is set forth in NCBI Ref. Nos. NM 001243539.1, NM_001256765.1, NM_002189.3 or NM_172200.2. In some embodiments, the IL-15Ra protein includes an amino acid sequence having at least 90%, such as at least 90%, 91%, 92%, 93%, 94%, 95%, 96%, 97%, 98%, 99% or 100%, sequence identity to SEQ ID NO:18, preferably the amino acid sequence of SEQ ID NO:18 as set forth in FIG. 5.

In some embodiments, the fusion protein comprising an IL-15 protein and an IL-15 receptor alpha (IL-15Rα) protein include an amino acid sequence having at least 90%, such as at least 90%, 91%, 92%, 93%, 94%, 95%, 96%, 97%, 98%, 99% or 100%, sequence identity to SEQ ID NO:17, preferably the amino acid sequence of SEQ ID NO:17 as set forth in FIG. 5.

The exogenous polynucleotide encoding an IL-15 protein or a fusion protein comprising an IL-15 protein and an IL-15 receptor alpha (IL-15Rα) can be integrated into a genomic site by way of genomic editing.

### VII. Combined Artificial Cell Death/Reporter System Polypeptides

In some embodiments, an iPSC or derivative cell thereof described herein is modified to express exogenous combined artificial cell death/reporter system polypeptides. In some embodiments, described herein is an iPSC or derivative cell thereof that expresses exogenous combined artificial cell death/reporter system polynucleotides. In some embodiments, provided herein is a polynucleotide encoding a combined artificial cell death/reporter polypeptide and an iPSC or derivative thereof engineered to express. In some embodiments, provided herein is a combined artificial cell death/reporter polypeptide and an iPSC or derivative thereof engineered to express.

A combined artificial cell death/reporter polypeptide acts as a safety switch so the cells can be killed if the patient has an adverse reaction. In some embodiments, the polypeptide or components thereof are useful for imaging such as, but not limited to, molecular imaging and PET imaging. It is advantageous to engineer cells to include a safety switch to eliminate the cells that have been infused into a patient in case of adverse events.

In some embodiments, provided is a combined artificial cell death/reporter polypeptide that can function as an artificial cell death polypeptide, a reporter polypeptide, or both an artificial cell death polypeptide and a reporter polypeptide. Having the combined artificial cell death and reporter polypeptide in a single polynucleotide that can be expressed as a single polypeptide has the advantage of reducing the number of gene edits of the cell. Descriptions of combined artificial cell death/reporter polypeptides can be found, for example, in US2022/0332782, the contents of which are incorporated herein by reference in its entirety.

In some embodiments, an artificial cell death/reporter polypeptide comprises an amino acid sequence having at least 90%, 91%, 92%, 93%, 94%, 95%, 96%, 97%, 98%, 99% or 100% sequence identity to a sequence selected from the group consisting of SEQ ID NO:25. Also provided is a polynucleotide encoding the artificial cell death/reporter polypeptide comprising an amino acid sequence having at least 90%, 91%, 92%, 93%, 94%, 95%, 96%, 97%, 98%, 99% or 100% sequence identity to a sequence selected from the group consisting of SEQ ID NO:25.

In some embodiments, an artificial cell death/reporter polypeptide comprises an amino acid sequence having at least 90%, 91%, 92%, 93%, 94%, 95%, 96%, 97%, 98%, 99% or 100% sequence identity to a sequence selected from the group consisting of SEQ ID NOS:27, 30 and 31. Also provided is a polynucleotide encoding the artificial cell death/reporter polypeptide comprising an amino acid sequence having at least 90%, 91%, 92%, 93%, 94%, 95%, 96%, 97%, 98%, 99% or 100% sequence identity to a sequence selected from the group consisting of SEQ ID NO:31.

In some embodiments, an artificial cell death/reporter polypeptide comprises nucleic acid sequence having at least 90%, 91%, 92%, 93%, 94%, 95%, 96%, 97%, 98%, 99% or 100% sequence identity to a sequence selected from the group consisting of SEQ ID NOS:26, 28 and 32. Also provided is a polynucleotide encoding the artificial cell death/reporter polypeptide comprising an amino acid sequence having at least 90%, 91%, 92%, 93%, 94%, 95%, 96%, 97%, 98%, 99% or 100% sequence identity to a sequence selected from the group consisting of SEQ ID NO:32.

In some embodiments, a combined artificial cell death/reporter polypeptide includes an HSV-TK fused to a truncated variant PSMA polypeptide via a linker. In some embodiments, a combined artificial cell death/reporter polypeptide comprises (1) an intracellular domain having a herpes simplex virus thymidine kinase (HSV-TK) and a linker, (2) a transmembrane region, and (3) an extracellular domain comprising a prostate-specific membrane antigen (PSMA) extracellular domain or fragment thereof. In some embodiments, the linker includes a Whitlow linker, an autoprotease peptide sequence, such as an autoprotease peptide sequence selected from the group consisting of porcine teschovirus-1 2A (P2A), thosea asigna virus 2A (T2A), equine rhinitis A virus 2A (E2A), foot-and-mouth disease virus 18 2A (F2A), and any linker described. In some embodiments, the artificial cell death/reporter polypeptide includes an intracellular domain of HSV-TK fused to a truncated variant PSMA polypeptide via a linker. As such, the PSMA portion is extracellular and the HSV-TK is located intracellular. In some embodiments, provided is a polynucleotide encoding a combined artificial cell death/reporter polypeptide and an iPSC or derivative thereof engineered to express.

In some embodiments, an artificial cell death polypeptide comprises a viral enzyme that is recognized by an antiviral drug. In some embodiments, the viral enzyme is a herpes simplex virus thymidine kinase (HSV-TK) (see, e.g., Bonini et al., Science, 1997 June 13; 276(5319):1719-24). In some embodiments, an HSV-TK polypeptide includes an amino acid sequence having at least 90%, 91%, 92%, 93%, 94%, 95%, 96%, 97%, 98%, 99% or 100% sequence identity to SEQ ID NO:23 or 29. Also provided is a polynucleotide encoding an HSV-TK polypeptide comprising an amino acid sequence having at least 90%, 91%, 92%, 93%, 94%, 95%, 96%, 97%, 98%, 99% or 100% sequence identity to a sequence selected from the group consisting of SEQ ID NO:23 or 29.

In some embodiments, the artificial cell death/reporter system polypeptide is encoded by an HSV-TK-T2A-PSMA transgene. In some embodiments, it is encoded by an HSV-TK (H168A)-T2A-PSMA transgene. In some embodiments, it is encoded by an HSV-TK (A168H)-T2A-PSMA transgene.

In some embodiments, such cells have been engineered to include a gene for an artificial cell death polypeptide (a “suicide gene”) which is a genetically encoded molecule that allows selective destruction of the cells (e.g., allowing selective ablation of the gene modified cells), thereby preventing collateral damage to contiguous cells and/or tissues. An artificial cell death polypeptide includes an engineered protein designed to prevent potential toxicity or otherwise adverse effects of a cell therapy. The artificial cell death/reporter polypeptide could mediate induction of apoptosis, inhibition of protein synthesis, DNA replication, growth arrest, transcriptional and post-transcriptional genetic regulation and/or antibody-mediated depletion. In some embodiments, provided herein is an artificial cell death polypeptide. In some embodiments, provided herein is a polynucleotide encoding an artificial cell death and an iPSC or derivative thereof engineered to express. In some instance, the artificial cell death polypeptide is activated by an exogenous molecule, e.g., an antibody, anti-viral drug, or radioisotopic conjugate drug, that when activated, triggers apoptosis and/or cell death of a therapeutic cell.

A reporter polypeptide refers to and includes an engineered protein that, in combination with an imaging probe, can be used to mark cells. In some embodiments, a reporter polypeptide comprises an antigen targeted by an entity, such as a small molecule compound, a radioisotopic conjugate, or an antibody or an antigen binding fragment thereof. In certain embodiments, the antigen is a prostate-specific membrane antigen (PSMA) polypeptide, also referred to as glutamate carboxypeptidase 2. PSMA is a type II membrane protein that is targeted to the secretary pathway by its transmembrane domain, which biochemically resembles a signal sequence without being cleaved. In various embodiments, the reporter polypeptide comprises a prostate-specific membrane antigen (PSMA) extracellular domain or fragment thereof.

In some embodiments, the PSMA polypeptide is a truncated variant as described WO2015143029A1 and WO2018187791A1, the disclosures of which are incorporated herein by reference in their entirety. In many embodiments, the prostate-specific membrane antigen (PSMA) polypeptide comprises or consists of an amino acid sequence at least 90%, such as at least 90%, 91%, 92%, 93%, 94%, 95%, 96%, 97%, 98%, 99% or 100%, identical to SEQ ID NO:24, preferably the amino acid sequence of SEQ ID NO:24. In many embodiments, the PSMA antigen may also function as an artificial cell death polypeptide since expression of truncated PSMA in a cell induces cell death of the engineered cell when the cell is contacted with a radioisotopic conjugate drug that binds to PSMA via a peptide. PSMA-targeting compounds are described in WO2010/108125, the disclosure of which is incorporated herein by reference. In some embodiments, a truncated variant PSMA polypeptide comprises an amino acid sequence having at least 90%, 91%, 92%, 93%, 94%, 95%, 96%, 97%, 98%, 99% or 100% sequence identity to SEQ ID NO:24. In some embodiments, described is a polynucleotide encoding a truncated variant PSMA polypeptide comprises an amino acid sequence having at least 90%, 91%, 92%, 93%, 94%, 95%, 96%, 97%, 98%, 99% or 100% sequence identity to SEQ ID NO:24.

In some embodiments, the artificial cell death/reporter polypeptide comprises a viral enzyme that is recognized by an antiviral drug. In certain embodiments, the viral enzyme is a herpes simplex virus thymidine kinase (HSV-TK). In certain embodiments, the HSV-TK comprises an amino acid sequence at least 90%, such as at least 90%, 91%, 92%, 93%, 94%, 95%, 96%, 97%, 98%, 99% or 100%, identical to SEQ ID NO:23, preferably the amino acid sequence of SEQ ID NO:23. This enzyme phosphorylates the nontoxic prodrugs acyclovir or ganciclovir, which then become phosphorylated by endogenous kinases to GCV-triphosphate, causing chain termination and single-strand breaks upon incorporation into DNA, thereby killing dividing cells. In some embodiments, described is a polynucleotide encoding an HSV-TK polynucleotide encoding an amino acid sequence at least 90%, such as at least 90%, 91%, 92%, 93%, 94%, 95%, 96%, 97%, 98%, 99% or 100%, identical to SEQ ID NO:23.

In some embodiments, expression of the viral enzyme in an engineered immune cell expressing a chimeric antigen receptor (CAR) induces cell death of the engineered immune cell when the cell is contacted with one or more antiviral drugs. In certain embodiments, the one or more antiviral drugs comprise acyclovir or a derivative thereof, or ganciclovir or a derivative thereof.

In some embodiments, the cell expressing the artificial cell death/reporter system also expresses one or more of the other exogenous polypeptides described. In some instances, the cell expresses a CAR. In some instances, the cell expresses a CD16 polypeptide such as a CD16 variant. In some instances, the cell expresses an NKG2D polypeptide.

### VIII. Other Exogenous Polypeptides

In some embodiments, the genomic editing at one or more selected genomic sites described may include insertions of one or more exogenous polynucleotides encoding any of polypeptides including, but not limited to, artificial cell death polypeptides, targeting modalities, receptors, signaling molecules, transcription factors, pharmaceutically active proteins and peptides, drug target candidates, or proteins promoting engraftment, trafficking, homing, viability, self-renewal, persistence, and/or survival of genome-engineered iPSCs or derivative cells thereof.

Other exogenous polynucleotides encoding polypeptides may include those encoding PET reporters, homeostatic cytokines, and inhibitory checkpoint inhibitory proteins such as PD1, PD-L1, and CTLA4 as well as proteins that target the CD47/signal regulatory protein alpha (SIRPα) axis. In some embodiments, the genome-engineered iPSCs generated using the methods provided herein contain an insertion or deletion (in/del) modification at one or more endogenous genes associated with targeting modality, receptors, signaling molecules, transcription factors, drug target candidates, immune response regulation and modulation, or proteins suppressing engraftment, trafficking, homing, viability, self-renewal, persistence, and/or survival of the iPSCs or derivative cells thereof.

In some embodiments, the genome-engineered iPSCs generated using the methods provided contain one or more different exogenous polynucleotides encoding proteins comprising caspase, thymidine kinase, cytosine deaminase, CD20, ErbB2 or CD79b such that when the genome-engineered iPSCs contain two or more suicide genes, the suicide genes are integrated in different safe harbor loci such as, but not limited to, an AAVS1 locus, a CCR5 locus, a ROSA26 locus, a collagen locus, an HTRP locus, a beta-2 microglobulin locus, a GAPDH locus, a TCR locus and a RUNX1 locus.

### IX. Gene Locus

In one aspect, any of the exogenous polynucleotides described can be integrated into a specific gene locus selected from the group consisting of: an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus. In some embodiments, any of the exogenous polynucleotide constructs described can be integrated into a specific gene locus selected from the group consisting of: an AAVS1 locus, a B2M locus, a CIITA locus, a CD70 locus, a CLYBL locus, an NKG2A locus, an NKG2D locus, a TAP1 locus, a TAP2 locus, a TAPBP locus, a TRAC locus, a TRBC1 locus, a RFXANK locus, a RFX5 locus, and a RFXAP locus, and optionally the integration into the gene locus disrupts (such as, reduces or eliminates) expression of the gene. In some embodiments, any of the exogenous polynucleotide is integrated into a gene locus selected from the group consisting of an AAVS1 locus, a B2M locus, a CIITA locus, a CD70 locus, a CLYBL locus, an NKG2A locus and a TRAC locus, and optionally the integration into the gene locus disrupts (such as, reduces or eliminates) expression of the gene.

In other words, integration of an exogenous polynucleotide described can produce a disruption of one or more genes selected from the group consisting of an AAVS1 gene, a B2M gene, a CIITA gene, a CD70 gene, a CLYBL gene, an NKG2A gene, an NKG2D gene, a TAP1 gene, a TAP2 gene, a TAPBP gene, a TRAC (TRA) gene, a TRBC1 (TRB) gene, a RFXANK gene, a RFX5 gene, a RFXAP gene, and any combination thereof. In some instances, the disruption is to the B2M gene. In some instances, the disruption is to the CIITA gene. In some instances, the disruption is to the B2M gene and the CIITA gene.

In some embodiments, the exogenous polynucleotide encoding the CD16 protein or variant thereof and the NKG2D protein or variant thereof is integrated into any of the gene locus described. In some embodiments, the exogenous polynucleotide encoding HLA-E is integrated into any of the gene locus described. In some embodiments, the exogenous polynucleotide encoding HLA-G is integrated into any of the gene locus described. In some embodiments, the exogenous polynucleotide encoding IL-15 is integrated into any of the gene locus described. In some embodiments, the exogenous polynucleotide encoding IL-15Ra is integrated into any of the gene locus described. In some embodiments, the exogenous polynucleotide encoding an fusion protein containing IL-15 and IL-15Ra is integrated into any of the gene locus described.

### X. Targeted Genome Editing at a Selected Gene Locus

In some aspect, one or more of the exogenous polynucleotides described are integrated at one or more loci on the chromosome of a cell such as an iPSC. In some instances, the integration of the exogenous polynucleotide into the gene locus is produced by way of targeted genome editing. Non-limiting examples of targeted genome editing include any method selected from the group consisting of a CRISPR method, a zinc finger nuclease method, a TALEN method, a homing nuclease method, a homology recombination method, and any functional variation thereof.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.”

Available endonucleases capable of introducing specific and targeted DSBs include, but not limited to, zinc-finger nucleases (ZFN), transcription activator-like effector nucleases (TALEN), RNA-guided CRISPR (Clustered Regular Interspaced Short Palindromic Repeats) systems. Additionally, DICE (dual integrase cassette exchange) system utilizing phiC31 and Bxbl integrases is also a promising tool for targeted integration.

ZFNs are targeted nucleases comprising a nuclease fused to a zinc finger DNA binding domain. By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A “designed” zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A “selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the Fokl nuclease with a zinc finger DNA binding domain.

A TALEN is a targeted nuclease comprising a nuclease fused to a TAL effector DNA binding domain. By “transcription activator-like effector DNA binding domain”, “TAL effector DNA binding domain”, or “TALE DNA binding domain” it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US2011/0145940, which is herein incorporated by reference. The most recognized example of a TALEN in the art is a fusion polypeptide of the Fokl nuclease to a TAL effector DNA binding domain.

Another example of a targeted nuclease that finds use in the subject methods is a targeted Spoll nuclease, a polypeptide comprising a Spoll polypeptide having nuclease activity fused to a DNA binding domain, e.g. a zinc finger DNA binding domain, a TAL effector DNA binding domain, etc. that has specificity for a DNA sequence of interest. See, for example, U.S. 61/555,857, the disclosure of which is incorporated herein by reference.

Additional examples of targeted nucleases suitable for the present application include, but not limited to Bxbl, phiC3 1, R4, PhiBTl, and Wp/SPBc/TP90l-1, whether used individually or in combination.

Other non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like. As an example, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. As another example, CRISPR/Cpf1 comprises two major components: (1) a Cpf1 endonuclease and (2) a crRNA. When co-expressed, the two components form a ribonucleoprotein (RNP) complex that is recruited to a target DNA sequence comprising PAM and a seeding region near PAM. The crRNA can be combined to form a chimeric guide RNA (gRNA) to guide Cpf1 to target selected sequences. These two components can then be delivered to mammalian cells via transfection or transduction.

MAD7 is an engineered Cas12a variant originating from the bacterium Eubacterium rectale that has a preference for 5′-TTTN-3′ and 5′-CTTN-3′ PAM sites and does not require a tracrRNA. See, for example, WO2018/236548, the disclosure of which is incorporated herein by reference. Additional descriptions of CRISPR-MAD7 methods can be found in, e.g., CRISPR J., April 2020, 3(2):97-108.

DICE mediated insertion uses a pair of recombinases, for example, phiC31 and Bxbl, to provide unidirectional integration of an exogenous DNA that is tightly restricted to each enzymes' own small attB and attP recognition sites. Because these target att sites are not naturally present in mammalian genomes, they must be first introduced into the genome, at the desired integration site. See, for example, US2015/0140665 and Farriggo et al., Methods Mol Biol, 2017, 1642:69-85, the disclosures of which are incorporated herein by reference.

In one aspect, provided herein a construct comprising one or more exogenous polynucleotides for targeted genome integration. In some embodiments, the construct further comprises a pair of homologous arm specific to a desired integration site, and the method of targeted integration comprises introducing the construct to cells to enable site specific homologous recombination by the cell host enzymatic machinery. In another embodiment, the method of targeted integration in a cell comprises introducing a construct comprising one or more exogenous polynucleotides to the cell, and introducing a ZFN expression cassette comprising a DNA-binding domain specific to a desired integration site to the cell to enable a ZFN-mediated insertion. In yet another embodiment, the method of targeted integration in a cell comprises introducing a construct comprising one or more exogenous polynucleotides to the cell, and introducing a TALEN expression cassette comprising a DNA-binding domain specific to a desired integration site to the cell to enable a TALEN-mediated insertion. In another embodiment, the method of targeted integration in a cell comprises introducing a construct comprising one or more exogenous polynucleotides to the cell, introducing a Cpf1 expression cassette, and a gRNA comprising a guide sequence specific to a desired integration site to the cell to enable a Cpf1-mediated insertion. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion. In still another embodiment, the method of targeted integration in a cell comprises introducing a construct comprising one or more “att” sites of a pair of DICE recombinases to a desired integration site in the cell, introducing a construct comprising one or more exogenous polynucleotides to the cell, and introducing an expression cassette for DICE recombinases, to enable DICE-mediated targeted integration.

Sites for targeted integration include, but are not limited to, genomic safe harbors, which are intragenic or extragenic regions of the human genome that, theoretically, are able to accommodate predictable expression of newly integrated DNA without adverse effects on the host cell or organism. In certain embodiments, the genome safe harbor for the targeted integration is one or more loci of genes selected from the group consisting of AAVS1, CCR5, ROSA26, HTRP, GAPDH, TCR and RUNX1 genes. In some embodiments, a TCR gene is selected from the group consisting of a TRA gene, a TRB gene, a TRD gene, and a TRG gene.

In other embodiments, the site for targeted integration is selected for deletion or reduced expression of an endogenous gene at the insertion site. A deletion respect to expression of a gene includes any genetic modification that abolishes the expression of the gene. Examples of a deletion of expression of a gene include, e.g., a removal or deletion of a DNA sequence of the gene, an insertion of an exogenous polynucleotide sequence at a locus of the gene, and one or more substitutions within the gene, which abolishes the expression of the gene.

Genes for target deletion include, but are not limited to, genes of major histocompatibility complex (MHC) class I and MHC class II proteins. Multiple MHC class I and class II proteins must be matched for histocompatibility in allogeneic recipients to avoid allogeneic rejection problems. MHC deficient, including MHC-class I deficient, or MHC-class II deficient, or both, refers to cells that either lack, or no longer maintain, or have reduced level of surface expression of a complete MHC complex comprising a MHC class I protein heterodimer and/or a MHC class II heterodimer, such that the diminished or reduced level is less than the level naturally detectable by other cells or by synthetic methods. MHC class I deficiency can be achieved by functional deletion of any region of the MHC class I locus (chromosome 6p2l), or deletion or reducing the expression level of one or more MHC class-I associated genes including, not being limited to, beta-2 microglobulin (B2M) gene, TAP1 gene, TAP2 gene and tapasin genes. For example, the B2M gene encodes a common subunit essential for cell surface expression of all MHC class I heterodimers. B2M null cells are MHC-I deficient. MHC class II deficiency can be achieved by functional deletion or reduction of MHC-II associated genes including, not being limited to, RFXANK, CIITA, RFX5 and RFXAP. CIITA is a transcriptional coactivator, functioning through activation of the transcription factor RFX5 required for class II protein expression. CIITA null cells are MHC-II deficient. In some embodiments, one or more of the exogenous polynucleotides are integrated at one or more loci of genes selected from the group consisting of B2M, TAP1, TAP2, Tapasin, RFXANK, CIITA, RFX5 and RFXAP genes to thereby delete or reduce the expression of the gene(s) with the integration.

In some embodiments, the exogenous polynucleotides are integrated at one or more loci on the chromosome of the cell, preferably the one or more loci are of genes selected from the group consisting of AAVS1, CCR5, ROSA26, HTRP, GAPDH, RUNX1, B2M, TAP1, TAP2, Tapasin, NLRC5, CIITA, RFXANK, CIITA, RFX5, RFXAP, TCRa constant region, TCRb constant region, NKG2A, NKG2D, CD38, CIS, CBL-B, SOCS2, PD1, CTLA4, LAG3, TIM3, or TIGIT genes, provided at least one of the one or more loci is of a MHC gene, such as a gene selected from the group consisting of B2M, TAP1, TAP2, Tapasin, RFXANK, CIITA, RFX5 and RFXAP genes. In many embodiments, the one or more exogenous polynucleotides are integrated at a locus of an MHC class-I associated gene, such as a B2M gene, TAP1 gene, TAP2 gene, or Tapasin gene; and at a locus of an MHC-II associated gene, such as a RFXANK, CIITA, RFX5, RFXAP, or CIITA gene; and optionally further at a locus of a safe harbor gene selected from the group consisting of AAVS1, CCR5, ROSA26, HTRP, GAPDH, TCR and RUNX1 genes. In some embodiments, a TCR gene is selected from the group consisting of a TRA gene, a TRB gene, a TRD gene, and a TRG gene. In various embodiments, the one or more of the exogenous polynucleotides are integrated at the loci of CIITA, AAVS1 and B2M genes.

In some embodiments, (i) an exogenous polynucleotide is integrated at a gene locus; (ii) a different exogenous polynucleotide is integrated at a locus of CIITA gene; and (iii) another different exogenous polynucleotide is integrated at a locus of B2M gene; wherein integrations of the exogenous polynucleotides delete or reduce expression of CIITA and B2M genes. In some embodiments, (i) an exogenous polynucleotide is integrated at a gene locus; (ii) another exogenous polynucleotide is integrated at a locus of CIITA gene; and (iii) yet another exogenous polynucleotide is integrated at a locus of B2M gene; wherein integrations of the exogenous polynucleotides eliminate or reduce expression of CIITA and B2M genes. In certain embodiments, (i) a first exogenous polynucleotide is integrated at a safe harbor locus; (ii) a second exogenous polynucleotide is integrated at a locus of CIITA gene; and (iii) a third exogenous polynucleotide is integrated at a locus of B2M gene; wherein integrations of the exogenous polynucleotides eliminate or reduce expression of CIITA and B2M genes. In some embodiments, (i) a first exogenous polynucleotide is integrated at a AAVS1, CCR5, ROSA26, HTRP, GAPDH, TRA, TRB, TRD, TRG or RUNX1 gene locus; (ii) a second exogenous polynucleotide is integrated at a CIITA gene locus; and (iii) a third exogenous polynucleotide is integrated at a B2M gene locus; wherein integrations of the exogenous polynucleotides eliminate or reduce expression of CIITA and B2M genes.

In some embodiments, an exogenous polynucleotide is integrated at a CD70 gene locus. In some embodiments, (i) an exogenous polynucleotide is integrated at a gene locus; (ii) a different exogenous polynucleotide is integrated at a CIITA gene locus; (iii) another different exogenous polynucleotide is integrated at a B2M gene locus; and (iv) yet another different exogenous polynucleotide is integrated at a CD70 gene locus; wherein integrations of the exogenous polynucleotides eliminate or reduce expression of the CD70, CIITA and B2M genes.

In some embodiments, (i) a first exogenous polynucleotide is integrated at a gene locus; (ii) a second exogenous polynucleotide is integrated at a CIITA gene locus; (iii) a third exogenous polynucleotide is integrated at a B2M gene locus; and (iv) a fourth exogenous polynucleotide is integrated at a CD70 gene locus; wherein integrations of the exogenous polynucleotides eliminate or reduce expression of the CD70, CIITA and B2M genes.

In certain embodiments, (i) a first exogenous polynucleotide is integrated at a safe harbor gene locus; (ii) a second exogenous polynucleotide is integrated at a CIITA gene locus; (iii) a third exogenous polynucleotide is integrated at a B2M gene locus; and (iv) a fourth exogenous polynucleotide is integrated at a CD70 gene locus; wherein integrations of the exogenous polynucleotides eliminate or reduce expression of the CD70, CIITA and B2M genes.

In some embodiments, (i) a first exogenous polynucleotide is integrated at a AAVS1, CCR5, ROSA26, HTRP, GAPDH, TRA, TRB, TRD, TRG or RUNX1 gene locus; (ii) a second exogenous polynucleotide is integrated at a CIITA gene locus; (iii) a third exogenous polynucleotide is integrated at a B2M gene locus; and (iv) a fourth exogenous polynucleotide is integrated at a CD70 gene locus; wherein integrations of the exogenous polynucleotides eliminate or reduce expression of the CD70, CIITA and B2M genes.

### XI. Derivative Cells from iPSC Cells

In some aspects, provided is a cell differentiated from an iPSC cell or a derivative thereof. In some embodiments, iPSCs are differentiated into a cell type which is then cultured and differentiated into another cell type. For instance, an iPSC can be differentiated into a progenitor cell such as an NK progenitor cell, which is then cultured under conditions to become a mature cell such as an NK cell. As described above, the genomic edits introduced into the iPSC are retained in the derivative cell. In some embodiments of the derivative cell obtained from iPSC differentiation, the derivative cell is a hematopoietic cell, including, but not limited to, hematopoietic stem and progenitor cells (HSCs), hematopoietic multipotent progenitor cells, T cell progenitors, natural killer (NK) cell progenitors, B cell progenitors, CD34+ hematopoietic progenitor cells, T cells, NKT cells, NK cells, B cells, antigen presenting cells (APC), monocytes and macrophages. In some embodiments, the derivative cell is an immune effector cell, such as an NK cell or a T cell.

In some embodiments, the iPSC is produced from whole peripheral blood mononuclear cells. In some embodiments, the iPSC is produced from an NK cell. In some embodiments, the iPSC is produced from a T cell. In some embodiments, the iPSC is produced from a reprogrammed NK cell. In some embodiments, the iPSC is produced from a reprogrammed T cell. In some embodiments, the iPSC is produced from a reprogrammed whole peripheral blood mononuclear cell.

Also provided is a method of manufacturing the differentiated cell or a derivative thereof. In some embodiments, the method includes differentiating the iPSC under conditions to promote, facilitate or generate a specific differentiated cell. In some instances, the differentiated cells is further cultured to produce a cell derived from the differentiated cell, e.g., a derivative cell.

An iPSC can be differentiated by any method known in the art. Exemplary methods are described in U.S. Pat. Nos. 10,947,502; 8,846,395; 8,945,922; 8,318,491; WO2010/099539; WO2010/141801; WO2012/109208; WO2016/010148; WO2017/070333; WO2017/070337; WO2017/179720; WO2018/048828; and WO2019/157597 and WO2020/252477; the contents of which are herein incorporated by reference in their entireties. The differentiation protocol may use feeder cells or may be feeder-free. Feeder cells or feeders include cells of one type that are co-cultured with cells of a second type to provide an environment in which the cells of the second type can grow, expand, or differentiate, as the feeder cells provide stimulation, growth factors and nutrients for the support of the second cell type.

In one embodiment, the differentiated iPSCs are NK cells which are prepared by a method of differentiating an iPSC into an NK cell. In some embodiments, the iPSCs are subjected to a differentiation protocol including the addition of recombinant human IL-12p70 (e.g., IL-12) to the culture media for the final 24 hours of culture. By including the IL-12 in the differentiation protocol, cells that are primed with IL-12 demonstrate more rapid cell killing compared to those that are differentiated in the absence of IL-12. In addition, the cells differentiated using the IL-12 conditions demonstrate improved cancer cell growth inhibition. Descriptions of methods for generating iPSC-derived NK cells can be found, for example, in WO2010/099539; Euchner et al., Front Immunol, 2021 May 4, 12:640672; Li et al., Cell Stem Cell, 2018 Aug. 2, 23:181-192; and Karagiannis and Kim, Mol Cells, 2021 Aug. 31, 44(8):541-548. In many embodiments, recombinant human IL-12p70 (human IL-12) includes a IL-12 p40 subunit and/or a IL-12 p35 subunit. In many embodiments, a IL-12 p40 subunit is connected to a IL-12 p35 subunit by way of a linker which can be any of those described herein. In some embodiments, recombinant human IL-12p70 includes a IL-12 p40 subunit, a Whitlow linker, and a IL-12 p35 subunit. In some embodiments, the recombinant human IL-12p70 protein has at least 90%, such as at least 90%, 91%, 92%, 93%, 94%, 95%, 96%, 97%, 98%, 99% or 100%, sequence identity to SEQ ID NO:33. In some embodiments, the recombinant human IL-12p70 protein is encoded by a polynucleotide sequence having at least 90%, 91%, 92%, 93%, 94%, 95%, 96%, 97%, 98%, 99% or 100%, sequence identity to SEQ ID NO:34.

In many embodiments, the differentiated iPSCs are T cells which are prepared by a method of differentiating an iPSC into a T cell. Protocols for generating a T cell from an iPSC include those disclosed, for example, in WO2010/099539, WO2010/141801, WO2017/070333, WO2017/070337, WO2017/179720, WO2018/048828, WO2019/157597 and WO2020/252477, the contents of which are herein incorporated by reference in their entireties.

In one embodiment, the differentiated iPSCs are T cells which are prepared by a method of differentiating an iPSC into an T cell. In some embodiments, the iPSCs are subjected to a differentiation protocol including the addition of recombinant human DLL-4 protein to the culture media. In some cases, the cells are cultured in medium comprising human DLL-4 protein for the final hours (e.g., 12, 18, 20, or 24 hours) of culture. In some embodiments, recombinant human DLL-4 protein comprises an amino acid sequence having at least 90%, such as at least 90%, 91%, 92%, 93%, 94%, 95%, 96%, 97%, 98%, 99% or 100%, sequence identity to SEQ ID NO:35. In some embodiments, recombinant human DLL-4 comprises an amino acid sequence having at least 90%, such as at least 90%, 91%, 92%, 93%, 94%, 95%, 96%, 97%, 98%, 99% or 100%, sequence identity to SEQ ID NO:36. In some embodiments, recombinant human DLL-4 comprises an amino acid sequence having at least 90%, such as at least 90%, 91%, 92%, 93%, 94%, 95%, 96%, 97%, 98%, 99% or 100%, sequence identity to SEQ ID NO:37.

Provided herein is a recombinant DLL4 variant protein having an amino acid sequence selected from the group consisting of SEQ ID NOS:35-37. In some embodiments, the recombinant DLL4 variant protein has an amino acid sequence provided in SEQ ID NOS:90-95 of US2022/0333074, the contents including Table 2 and the sequence listing of which are herein incorporated by reference.

In many embodiments, the differentiated iPSCs are CD34-positive (CD34+) cells which are prepared by a method of differentiating an iPSC into a CD34+ cell. Protocols for generating a CD34+ cell from an iPSC include those disclosed, for example, in WO2010/099539, WO2010/141801, WO2017/070333, WO2017/070337, WO2018/048828, WO2019/157597 and WO2020/252477, the contents of which are herein incorporated by reference in their entireties.

In some embodiments, iPSC cells are differentiated into hematopoietic progenitor cells (HPCs). In an exemplary embodiment of a differentiation protocol, iPSC cells are in HDM-I media plus H1152. In some instances, HDM media contains IMDM medium, Ham's F12 medium, CTS B27 minus vitamin A supplement, non-essential amino acids, ascorbic acid, Mg 2-phosphate, monothioglycerol, and heparin. HDM-I media can contain HDM+CHIR99021 GSK3 inhibitor, FGF2, and VEGF. In some instances, the cells are further cultured in HDM-II medium comprising HDM media in addition to BMP4, FGF2, and VEGF. In some instances, the cells are further cultured in HDM-III medium comprising HDM in addition to BMP4, SCF, TPO, FLT3L, and IL3. The resulting HPCs can be collected.

In some embodiments, HPCs are differentiated to produce NK or T cells. In some instances, the HPCs are cultured in retronectin/DLL4-coated bioreactors, e.g., G-Rex bioreactors. Notch signaling factors, cytokines, and growth factors can be added to culture medium to facilitate differentiation into lymphoid lineage and subsequent NK or T cell maturation and activation.

In some embodiments, maturation and/or activation of NK or T cells from HPCs includes culturing the HPCs in a culture medium comprising a recombinant IL-12 protein. IL-12 is a cytokine that stimulates the production of interferon-gamma (IFN-7) and tumor necrosis factor-alpha (TNF-α) from T cells and natural killer (NK) cells. In some embodiments, a recombinant IL-12 protein comprises human IL-12p70. In various embodiments, recombinant IL-12 comprises a human IL-12p70 p40 subunit and a human IL-12p70 p35 subunit. In various embodiments, recombinant IL-12 protein comprises a human IL-12p70 p40 subunit, a human IL-12p70 p35 subunit and a linker. In various embodiments, recombinant IL-12 protein comprises from N- to C-terminus: a human IL-12p70 p40 subunit, a linker, and a human IL-12p70 p35 subunit. In certain embodiments, recombinant IL-12 protein comprises from N- to C-terminus: a human IL-12p70 p35 subunit, a linker, and a human IL-12p70 p40 subunit. In some embodiments, recombinant IL-12 protein comprises from N- to C-terminus: a human IL-12p70 p40 subunit, a Whitlow linker, and a human IL-12p70 p35 subunit. In various embodiments, recombinant IL-12 protein comprises from N- to C-terminus: a human IL-12p70 p35 subunit, a Whitlow linker, and a human IL-12p70 p40 subunit. In some embodiments, a recombinant IL-12 protein comprises an amino acid sequence having at least 90%, at least 90%, 91%, 92%, 93%, 94%, 95%, 96%, 97%, 98%, 99% or 100%, sequence identity to SEQ ID NO:33. In various embodiments, a recombinant IL-12 protein is encoded by a polynucleotide having at least 90%, at least 90%, 91%, 92%, 93%, 94%, 95%, 96%, 97%, 98%, 99% or 100%, sequence identity to SEQ ID NO:34.

Detailed descriptions of generating iPSC-derived γδ T cells are known in the art and can be found in WO2022/216514. Briefly, peripheral blood mononuclear cells (PBMCs) from healthy donors can be reprogrammed into iPSCs using methods known in the art and described in WO2022/120334; WO2022/216514; WO2022/216624; WO2023/049918; U.S. Pat. Nos. 8,183,038; 8,268,620; 8,440,461; 8,546,140; 8,765,470; 8,952,801; 9,328,332; 9,499,786; 9,644,184; and 10,865,381; the contents of each are incorporated herein by reference in their entirety. The iPSCs can be differentiated into hematopoietic progenitor cells (HSCs) such as CD34+ HSCs using methods known in the art and described in WO2022/216514. The CD34+ HSCs can be further differentiated to generate T cells in particular, γδ T cells using methods known in the art and described in Examples 1 and 3 of WO2022/216514.

Detailed descriptions of generating iPSC-derived αβ T cells are known in the art and can be found in Example 2 of WO2023049918, the contents are incorporated herein by reference in its entirety.

In some embodiments, the iPSCs and derivatives thereof express a polynucleotide encoding the amino acid sequence of SEQ ID NO:1. In some embodiments, the iPSCs and derivatives thereof express a polynucleotide encoding the amino acid sequence of SEQ ID NO:2. In some embodiments, the iPSCs and derivatives thereof express a polynucleotide encoding the amino acid sequence of SEQ ID NO:4. In some embodiments, the iPSCs and derivatives thereof express a polynucleotide comprising the nucleic acid sequence of SEQ ID NO:6. In some embodiments, the iPSCs and derivatives thereof express a polynucleotide comprising the nucleic acid sequence of SEQ ID NO:7. In some embodiments, the iPSCs and derivatives thereof express a polynucleotide comprising the nucleic acid sequence of SEQ ID NO:9. In some embodiments, the iPSCs and derivatives thereof express a polynucleotide comprising the nucleic acid sequence of SEQ ID NO:13. In some embodiments, the iPSCs and derivatives thereof express a polynucleotide comprising the nucleic acid sequence of SEQ ID NO:39. In some embodiments, the iPSCs and derivatives thereof also express an IL-15 protein of SEQ ID NO:16. In particular embodiments, the iPSCs and derivatives thereof also express an IL-15 and IL-15Rα fusion protein of SEQ ID NO:17. In some instances, the iPSCs and derivatives thereof express an exogenous polynucleotide encoding an IL-15 protein of SEQ ID NO:16. In some instances, the iPSCs and derivatives thereof express an exogenous polynucleotide encoding an IL-15 and IL-15Rα fusion protein of SEQ ID NO:17. In some instances, the iPSCs and derivatives thereof express an exogenous polynucleotide comprising the nucleic acid sequence of SEQ ID NO:38. In some embodiments, the iPSCs and derivatives thereof also express an HLA-E protein of SEQ ID NO:14. In some embodiments, the iPSCs and derivatives thereof also express an HLA-G protein of SEQ ID NO:15. In some embodiments, the iPSCs and derivatives thereof also express an exogenous polynucleotide encoding an HLA-E protein of SEQ ID NO:14. In some embodiments, the iPSCs and derivatives thereof also express an exogenous polynucleotide encoding an HLA-G protein of SEQ ID NO:15. In some cases, the iPSCs and derivatives thereof express the polypeptide of SEQ ID NO: 19. In some cases, the iPSCs and derivatives thereof express the polypeptide of SEQ ID NO:20. In some cases, the iPSCs and derivatives thereof express the polynucleotide of SEQ ID NO:21 and/or SEQ ID NO:22. In some embodiments, the iPSCs and derivatives thereof express a chimeric antigen receptor described herein and in WO2022/120334, the contents are herein incorporated by reference in its entirety. In some embodiments, iPSCs and derivatives thereof express an artificial cell death polypeptide described herein and in US2022/0332782, the contents are herein incorporated by reference in its entirety. In certain embodiments, the iPSCs and derivatives thereof express a polypeptide comprising an amino acid sequence of SEQ ID NO:23, 24, 25, 27, 29, 30, or 31. In other embodiments, the iPSCs and derivatives thereof express a polynucleotide comprising a nucleic acid sequence of SEQ ID NO:26, 28 or 32. The iPSCs and derivatives thereof include, but are not limited to, human iPSCs generated from reprogrammed whole blood mononuclear cells, human iPSCs generated by reprogrammed NK cells, human iPSCs generated by reprogrammed T cells, CD34+ hematopoietic progenitor cells derived from human iPSCs, NK cells derived from human iPSCs, T cells derived from human iPSCs, NK cells differentiated from CD34+ hematopoietic progenitor cells derived from human iPSCs, and T cells differentiated from CD34+ hematopoietic progenitor cells derived from human iPSCs. The T cells can be αβ T cells or γδ T cells.

### XII. Vectors

In some aspects, provided is an isolated vector (construct) comprising a polynucleotide sequence encoding a useful polypeptide according to embodiments of the disclosure. Any vector known to those skilled in the art can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a recombinant protein in the cell. Conventional cloning techniques or artificial gene synthesis can be used to generate a recombinant expression vector according to embodiments described.

In some embodiments, any of the exogenous polynucleotides are operatively linked to one or more exogenous promoters such as, but not limited to, CAG, CMV, EF1a, PGK1, SV40, UBC and human beta actin, as well as other constitutive, inducible, temporal-specific, tissue-specific, and cell type-specific promoters. In some embodiments, any of the exogenous polynucleotides are operatively linked to one or more endogenous promoters found in a selected genomic site (e.g., gene locus) such as, but not limited to, AAVS1, CCR5, ROSA26, collagen, HTRP, beta-2 microglobulin (B2M), GAPDH, TCR (e.g., TRA, TRB, TRD and TRG) and RUNX1, as well as other locus that meet the criteria of a genome safe harbor.

In some embodiments, the vector comprises an exogenous polynucleotide having, in the 5′ to 3′ order, (a) a promoter; (b) a polynucleotide sequence; and (c) a terminator/polyadenylation signal. Non-limiting examples of constitutive promoters include CAG, EF1a, UBC, CMV, SV40, PGK1, and human beta actin. Non-limiting examples of terminator/polyadenylation signal include a SV40 signal, BGH signal, hGH signal, and PGK signal.

In some embodiment, the vector includes a left homology arm and a right homology arm flanking the exogenous polynucleotide. As used herein, “left homology arm” and “right homology arm” refers to a pair of nucleic acid sequences that flank an exogenous polynucleotide and facilitate the integration of the exogenous polynucleotide into a specified chromosomal locus. Sequences of the left and right arm homology arms can be designed based on the integration site of interest. In some embodiment, the left or right arm homology arm is homologous to the left or right side sequence of the integration site. In some embodiments, the left homology arm and a right homology arm target the CD70 gene.

### XIII. Compositions

In some aspects, provided are compositions or populations of iPSCs or derivative cells thereof that express one or more recombinant proteins described herein. In some embodiments, the cells are CD34+ cells, NK cells, T cells, iNK cells or iT cells. In certain embodiments, the cells are NK cells derived from iPSCs. In some embodiments, the cells are T cells derived from iPSCs. In some instances, the T cells derived from iPSCs are αβ T cells. In certain instances, the T cells derived from iPSCs are γδ T cells.

In some aspects, provided are compositions or populations of iPSCs or derivative cells thereof that can express one or more recombinant proteins including, but not limited to, a recombinant CD16 protein such as a CD16 variant protein, a recombinant NKG2D protein, a CAR protein, a fusion protein containing IL-15 and IL-15Ra, HLA-E, and HLA-G. In some embodiments, the compositions or populations of iPSCs or derivative cells thereof express a recombinant CD16 protein such as a CD16 variant protein, a recombinant NKG2D protein, and a CAR protein. In some embodiments, the compositions or populations of iPSCs or derivative cells thereof express a recombinant CD16 protein such as a CD16 variant protein, a recombinant NKG2D protein, a CAR protein, and a fusion protein containing IL-15 and IL-15Ra. In some embodiments, the compositions or populations of iPSCs or derivative cells thereof express recombinant CD16 protein such as a CD16 variant protein, a recombinant NKG2D protein, a CAR protein, and an IL-15 protein. In some embodiments, the compositions or populations of iPSCs or derivative cells thereof express a recombinant CD16 protein such as a CD16 variant protein, a recombinant NKG2D protein, a CAR protein, and either HLA-E, HLA-G or both HLA-E and HLA-G. In some embodiments, the compositions or populations of iPSCs or derivative cells thereof express a recombinant HSV-TK-PSMA fusion.

In some embodiments, the compositions include a population of either NK cells, T cells or CD34+ cells that have been differentiated from iPSCs and contain an exogenous polynucleotide construct encoding a CD16 protein (such as a high affinity CD16 variant), an NKG2D protein and a self-cleavage peptide. In some instances, the population of NK cells that have been differentiated from iPSCs contains an exogenous polynucleotide construct encoding a CD16 protein (such as a high affinity CD16 variant), an NKG2D protein and a self-cleavage peptide. In some embodiments, the population of either NK cells, T cells or CD34+ cells that have been differentiated from iPSCs contains an exogenous polynucleotide construct encoding a CD16 protein (such as a high affinity CD16 variant), an NKG2D protein and a self-cleavage peptide; and an exogenous polynucleotide construct encoding a CAR. In some embodiments, the population of either NK cells, T cells or CD34+ cells that have been differentiated from iPSCs contains an exogenous polynucleotide construct encoding a CD16 protein (such as a high affinity CD16 variant), an NKG2D protein and a self-cleavage peptide; and an exogenous polynucleotide construct encoding HLA-E, HLA-G or both. In some embodiments, the population of either NK cells, T cells or CD34+ cells that have been differentiated from iPSCs contains an exogenous polynucleotide construct encoding a CD16 protein (such as a high affinity CD16 variant), an NKG2D protein and a self-cleavage peptide and an exogenous polynucleotide construct encoding a fusion protein containing IL-15 and IL-15Ra. In some embodiments, the population of either NK cells, T cells or CD34+ cells that have been differentiated from iPSCs contains an exogenous polynucleotide construct encoding a CD16 protein (such as a high affinity CD16 variant), an NKG2D protein and a self-cleavage peptide and an exogenous polynucleotide construct encoding an IL-15 protein. In some embodiments, the population of either NK cells, T cells or CD34+ cells that have been differentiated from iPSCs contains an exogenous polynucleotide construct encoding a CD16 protein (such as a high affinity CD16 variant), an NKG2D protein and a self-cleavage peptide; an exogenous polynucleotide construct encoding a CAR; and an exogenous polynucleotide construct encoding HLA-E, HLA-G or both. In some embodiments, the population of either NK cells, T cells or CD34+ cells that have been differentiated from iPSCs contains an exogenous polynucleotide construct encoding a CD16 protein (such as a high affinity CD16 variant), an NKG2D protein and a self-cleavage peptide; an exogenous polynucleotide construct encoding a CAR; and an exogenous polynucleotide construct encoding a fusion protein containing IL-15 and IL-15Ra. In some embodiments, the population of either NK cells, T cells or CD34+ cells that have been differentiated from iPSCs contains an exogenous polynucleotide construct encoding a CD16 protein (such as a high affinity CD16 variant), an NKG2D protein and a self-cleavage peptide; an exogenous polynucleotide construct encoding a CAR; an exogenous polynucleotide construct encoding HLA-E, HLA-G or both; and an exogenous polynucleotide construct encoding a fusion protein containing IL-15 and IL-15Ra. In some embodiments, the population of either NK cells, T cells or CD34+ cells that have been differentiated from iPSCs contains an exogenous polynucleotide construct encoding a CD16 protein (such as a high affinity CD16 variant), an NKG2D protein and a self-cleavage peptide; an exogenous polynucleotide construct encoding a CAR; an exogenous polynucleotide construct encoding HLA-E, HLA-G or both; and an exogenous polynucleotide construct encoding an IL-15 protein. In some embodiments, any of the cell populations can also express an exogenous polynucleotide construct comprising HSV-TK-PMSA as described herein.

In some instances, one or more of the exogenous polynucleotide constructs have been introduced into a specific genomic site (e.g., gene locus). In many instances, one or more of the exogenous polynucleotide constructs have been introduced into a safe harbor genomic site (e.g., gene locus). In some instances, one or more of the exogenous polynucleotide constructs have been introduced into a gene locus such that the expression of the gene is reduced or eliminated.

In some aspects, provided is a composition comprising an isolated polynucleotide, a host cell and/or an iPSC or derivative cell thereof described herein. In certain embodiments, the composition also includes one or more therapeutic agents selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleotides of interest, an antibody, a chemotherapeutic agent, a radioactive moiety or agent, or an immunomodulatory drug.

In some embodiments, the composition comprises an isolated polynucleotide, a host cell and/or an iPSC or derivative cell thereof described herein and a pharmaceutically acceptable carrier. Non-limiting examples of a pharmaceutically acceptable carrier include any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration. A pharmaceutically acceptable carrier includes a non-toxic material that does not interfere with the effectiveness of a composition described herein or the biological activity of a composition described herein. The formulation of pharmaceutically active ingredients with pharmaceutically acceptable carriers is known in the art, e.g., Remington: The Science and Practice of Pharmacy (e.g. 21^{st} edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions described.

### XIV. Methods of Treating Cancer

In some aspects, provided are methods of treating a disease or disorder such as a cancer and/or an autoimmune disease by administering any of the cells described herein. The teachings of the present disclosure may be relevant to any and all cancers. In some embodiments, the cancer treated by methods of the present disclosure is a solid tumor. In some embodiments, the cancer treated by methods of the present disclosure is a hematologic malignancy.

In some embodiments, NK cells described herein that have been differentiated from iPSCs and engineered (e.g., modified) to express one or more of the exogenous polynucleotide constructs provided can be administered to a patient for treating cancer and/or an autoimmune disease. In some embodiments, T cells (e.g., gamma-delta T cells or γδ T cells) described herein that have been differentiated from iPSCs and engineered to express one or more of the exogenous polynucleotide constructs provided can be administered to a patient for treating cancer and/or an autoimmune disease. In some embodiments, CD34+ cells described herein that have been differentiated from iPSCs and engineered to express one or more of the exogenous polynucleotide constructs provided can be administered to a patient for treating cancer and/or an autoimmune disease. In many embodiments, the one or more of the exogenous polynucleotide constructs include, but are not limited to, a exogenous polynucleotide construct encoding a CD16 protein (such as a high affinity CD16 variant), an NKG2D protein and a self-cleavage peptide; an exogenous polynucleotide construct encoding a CAR; an exogenous polynucleotide construct encoding an HLA-E protein; an exogenous polynucleotide construct encoding an HLA-G protein; an exogenous polynucleotide construct encoding an HLA-E protein and an HLA-G protein; an exogenous polynucleotide construct encoding an HLA-E protein, an HLA-G protein, and a self-cleavage peptide; an exogenous polynucleotide construct encoding a fusion protein containing IL-15 and IL-15Ra; and an exogenous polynucleotide construct encoding an IL-15 protein. In various embodiments such cells can be administered to treat a patient with cancer, such as any type of cancer. In some embodiments, the cancer treated by methods of the present disclosure include a glioblastoma, ovarian cancer, cervical cancer, head and neck cancer, liver cancer, prostate cancer, pancreatic cancer, renal cell carcinoma, bladder cancer, other solid tumor cancer, or hematologic malignancy. In some embodiment, the hematologic malignancy is a leukemia (e.g., acute lymphocytic (ALL), chronic lymphocytic (CLL), acute myeloid (AML), chronic myeloid (CML)), myeloma, or lymphoma (e.g., Hodgkin's or non-Hodgkin's (NHL)).

In various embodiments of the treatment methods described herein, the disease is an autoimmune disease or disorder. In some embodiments, the autoimmune disease or disorder is rheumatoid arthritis (RA), multiple sclerosis (MS), Sjögren's syndrome, systemic lupus erythematosus, sarcoidosis, type 1 diabetes mellitus, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Myasthenia gravis, Hashimoto's thyroiditis, Graves' disease, chronic inflammatory demyelinating polyneuropathy, Guillain-Barre syndrome, Crohn's disease or ulcerative colitis.

In some embodiments, when more than one polypeptide with unique antigen-binding specificities are administered via cells described, the methods can be used to target multiple antigens (or multiple epitopes in the same antigen) in the same disease (e.g., tumor or autoimmune disease), or multiple antigens in different diseases (e.g., tumor or autoimmune disease). In various embodiments, when engineered cells expressing more than one polypeptide with unique antigen-binding specificities are administered, the methods as described can be used to target multiple antigens (or multiple epitopes in the same antigen) in the same disease (e.g., tumor or autoimmune disease), or multiple antigens in different diseases (e.g., tumor or autoimmune disease).

### XV. Examples

### Example 1: Exogenous Expression of CD16 in iPSC Cells Expressing a Chimeric Antigen Receptor (CAR) and Gamma-Delta iT Cells Derived Therefrom

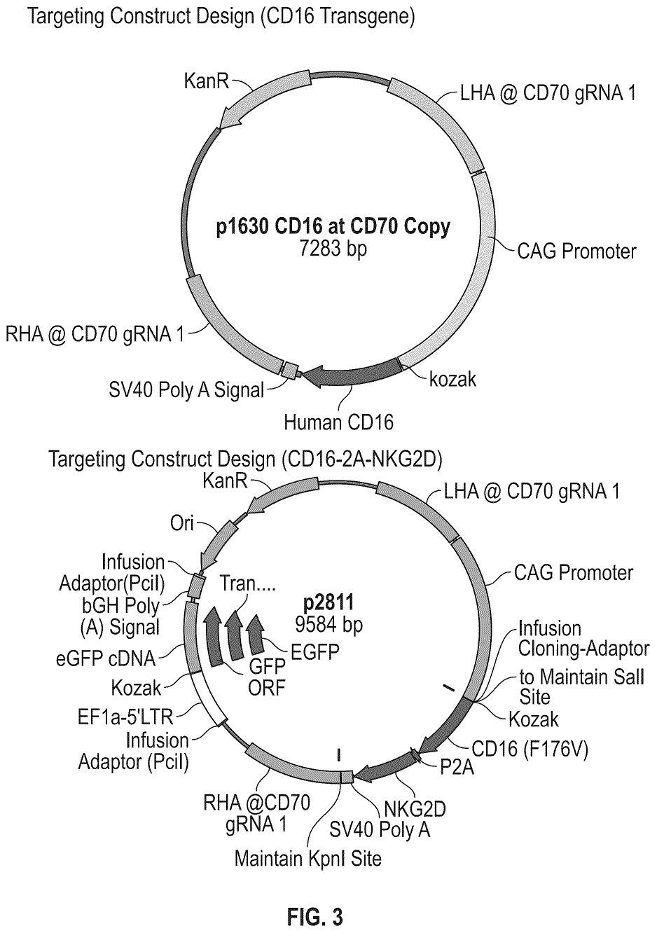

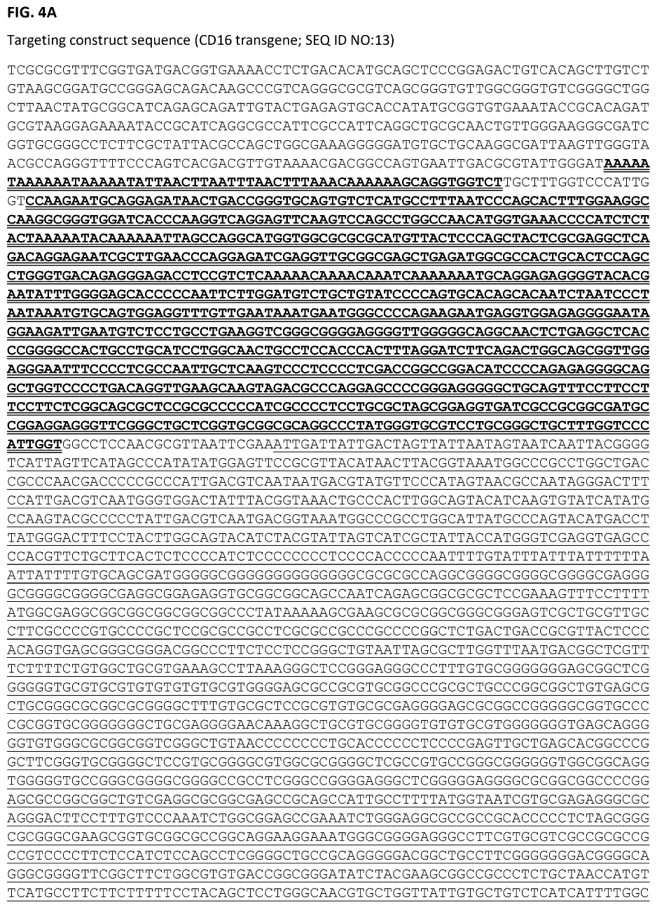

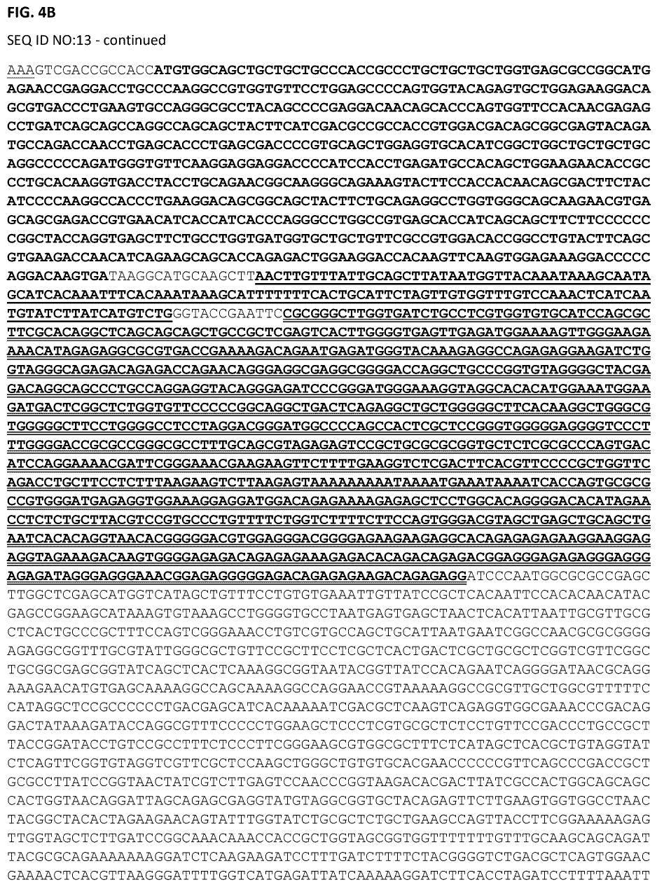

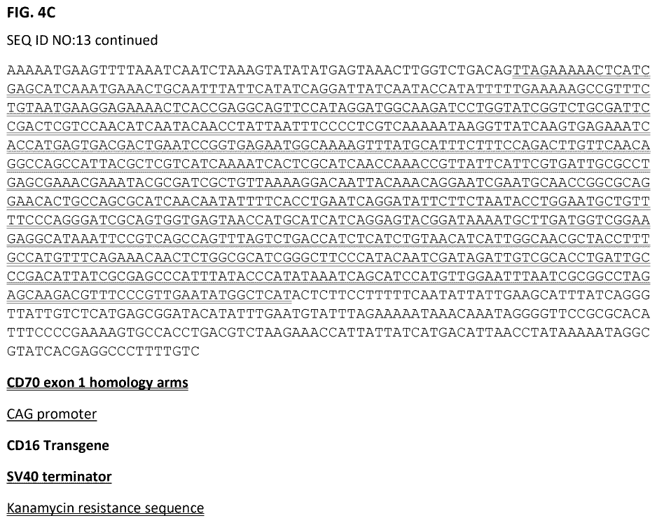

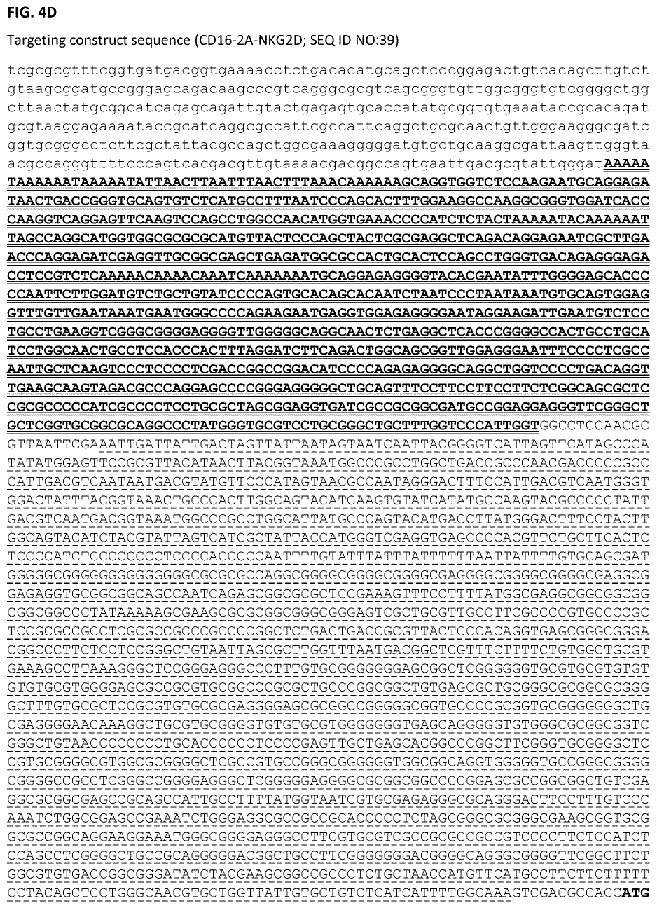

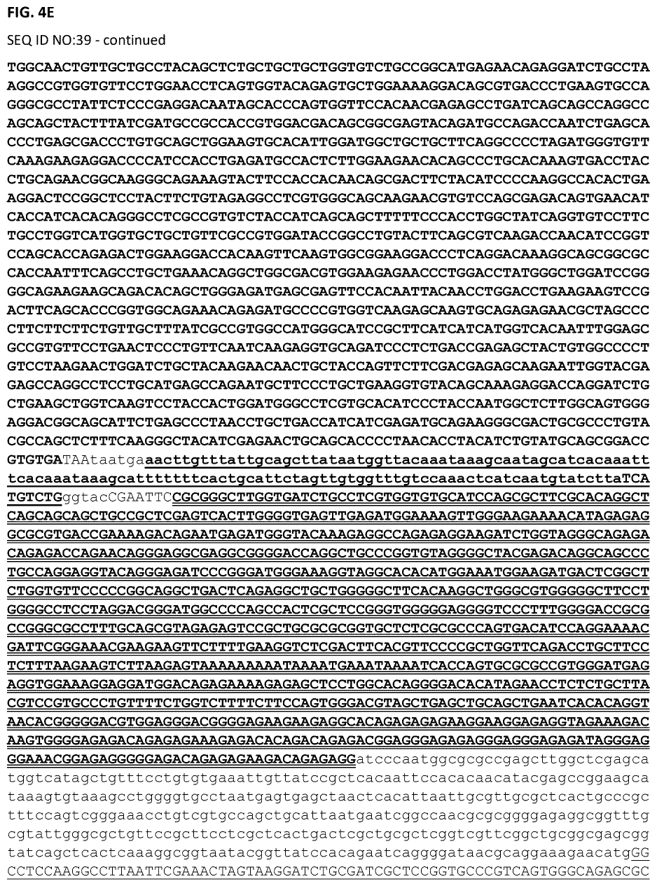

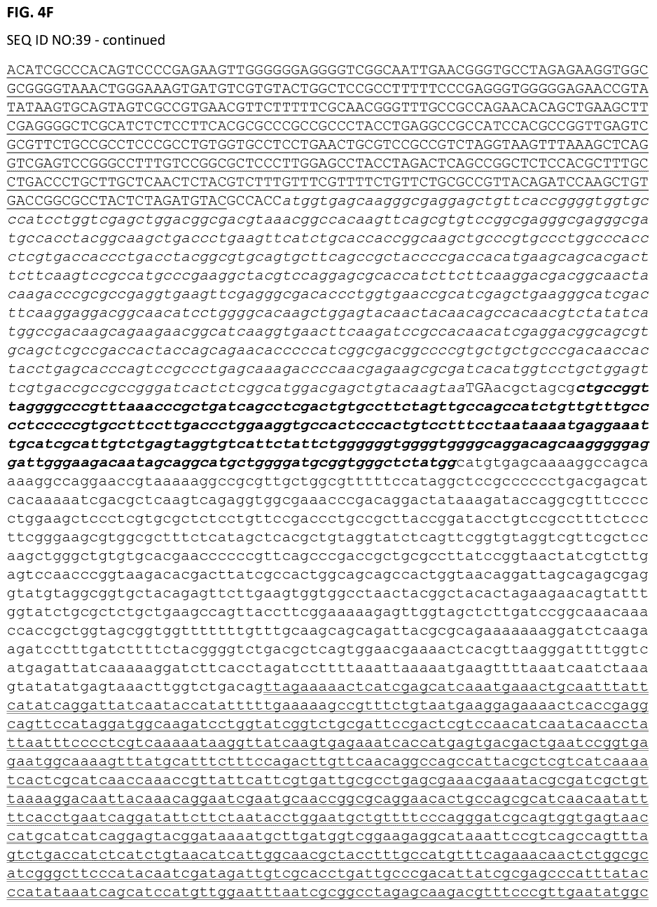

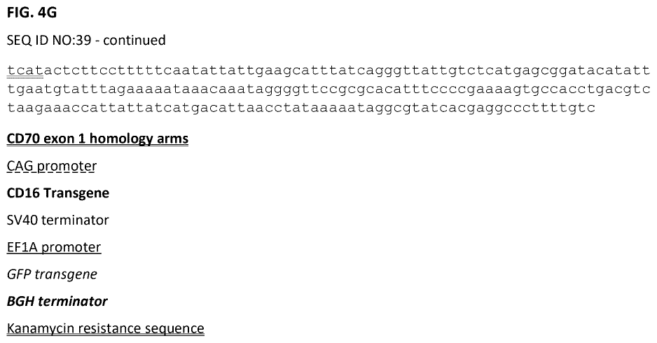

The CD16 transgene described herein was introduced into the CD70 locus of an iPSC cell carrying a CD19-specific CAR using homology directed repair and a CRISPR nuclease. An exemplary targeting construct is provided in FIG. 3 “p1630 CD16 at CD70” and SEQ ID NO:13. The resulting engineered iPSC cells were differentiated into gamma/delta iT-CAR cells (iPSC-derived 76 CAR T cells) as described below.

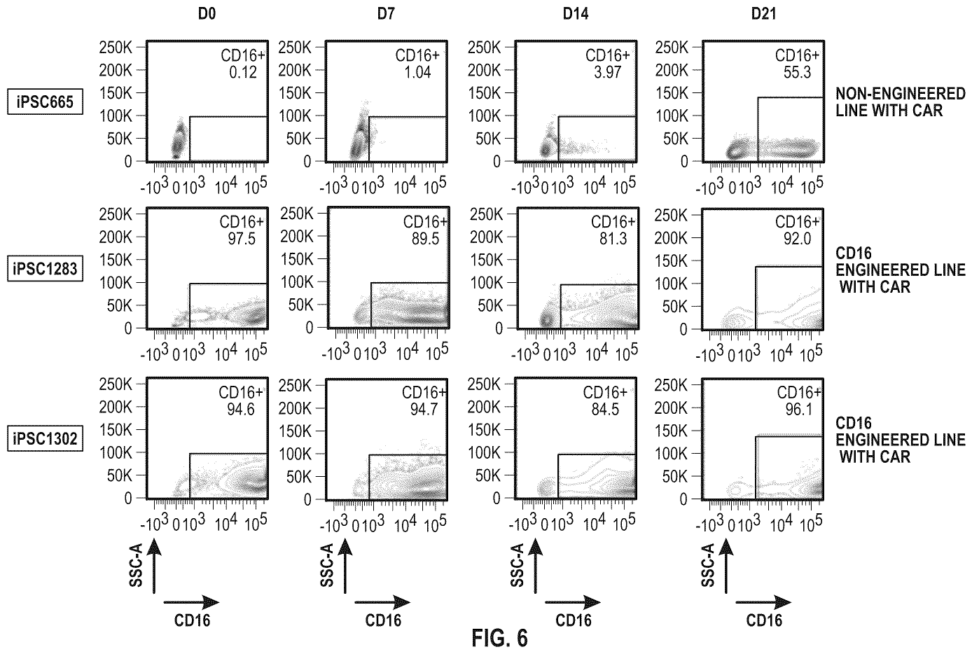

Detailed descriptions of useful methods for producing hematopoietic progenitor cells from iPSCs can be found in WO2022/120334, the contents including the examples are herein incorporated by reference in its entirety. iPSC derived hematopoietic progenitor cells (CD34-expressing HSCs) were differentiated into iPSC-derived γδ T cells on plates coated with retronectin and DLL4-Fc proteins and cultured in complete medium containing basal TCDM medium supplemented with SCF (50 ng/ml), FLT3L (50 ng/ml), IL-7 (50 ng/ml), and TPO (50 ng/ml). At day 7, the cells were cultured in complete medium supplemented with CHIR09921 (2 μM final concentration) on plates coated with RetroNectin® and DLL4-Fc proteins. At day 14, day 17, and day 21, the cells were re-seeded and cultured in complete medium supplemented with IL-2 (5 ng/ml) on plates coated with RetroNectin® and DLL4-Fc proteins. Culture medium was refreshed as needed. CD16 expression in the cells (see iPSC1283 and iPSC1302 of FIG. 6) was evaluated from day 0 to day 21.

### Example 2: Enhanced Anti-Tumor Activity of iNK Cells Overexpressing NKG2D Protein

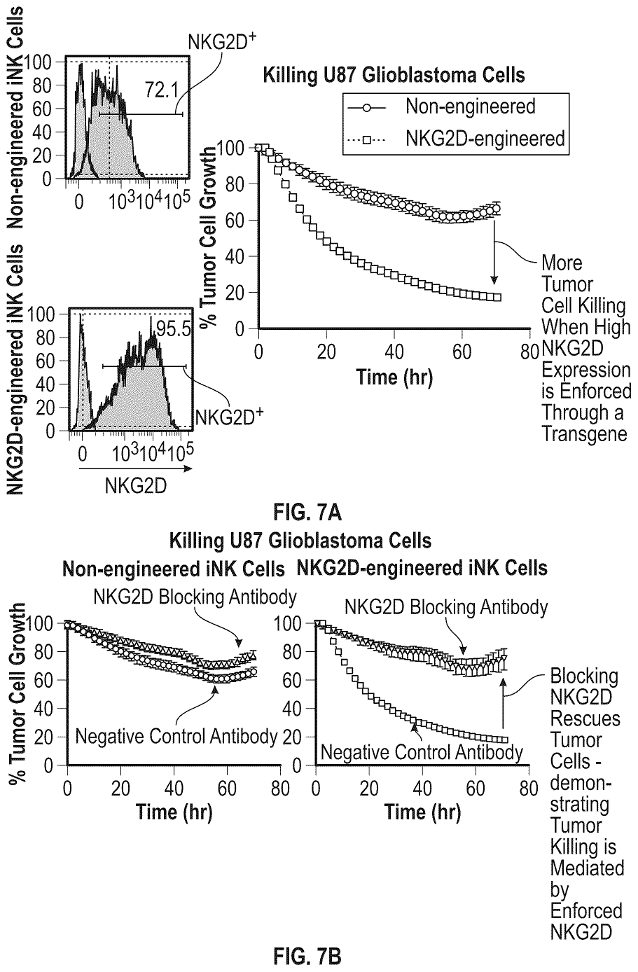

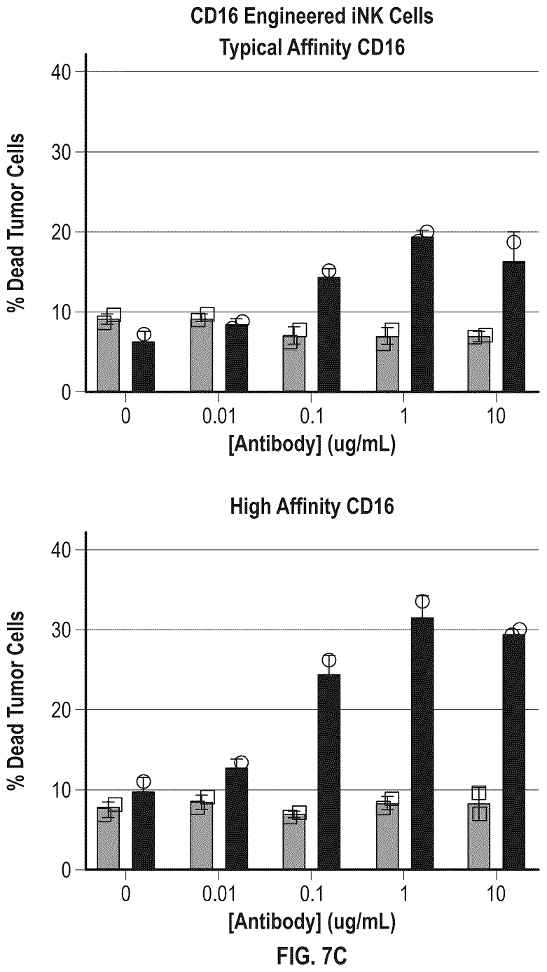

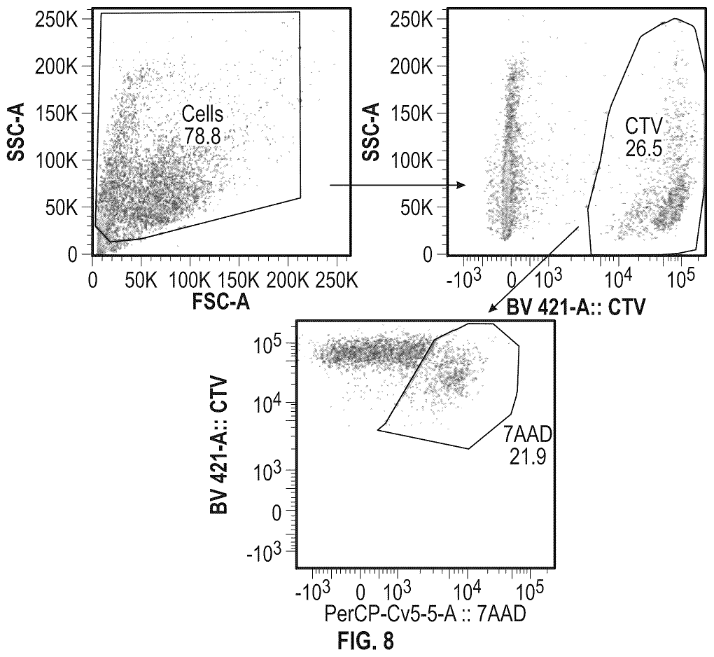

iPSCs were engineered to constitutively express NKG2D. When iPSCs were differentiated into iNK cells, expression of NKG2D was quantified by flow cytometry. The data demonstrated that NKG2D expression was increased to 95.5% of the engineered iNK cells compared to only 72.1% of the non-engineered iNK cells (FIG. 7A, left).

Non-engineered or NKG2D-engineered iNK cells were used in a killing assay with NLR-labeled U87 glioblastoma cells that express stress ligands (MIC-A and MIC-B) and can trigger NKG2D activity. The NKG2D-engineered iNK cells more potently killed U87 cells (FIG. 7A, right). To confirm that enhanced killing was due to NKG2D expression, a neutralizing (blocking) antibody against NKG2D was used in some conditions to block the interaction of NKG2D with stress the ligands on U87 cells. There was a marked reduction in U87 killing when the NKG2D neutralizing antibody was included with the NKG2D-engineered iNK cells (FIG. 7B). An isotype IgG1 control was used as an antibody control.

### Example 3: Enhanced Antibody-Dependent Cellular Cytotoxicity (ADCC) of iNK Cells Overexpressing High-Affinity CD16 Variant Protein

iPSCs were engineered to constitutively express one of two different naturally occurring variants of CD16 (a low affinity CD16 and a high affinity CD16). The iPSCs were then differentiated into iNK cells and used in a tumor killing assay where the targets were CD20+ lymphoblastic B cells. To trigger ADCC, an anti-CD20 therapeutic antibody rituximab (black bars) was included at various concentrations. As a negative control, non-binding isotype control antibody was used in some conditions (grey bars). When iPSCs were differentiated into iNK cells expressing the low affinity variant of CD16, ADCC was evident (increased dead tumor cells) only when rituximab was included (FIG. 7C; top panel). When iPSCs were differentiated into iNK cells expressing the high affinity variant of CD16, greater ADCC was observed compared to the low affinity version of CD16 (FIG. 7C; bottom panel).

### Example 4: Enhanced ADCC of CD16 Overexpressing iNK Cells Using a CAR-Mediated Tumor Killing Assay of Fluorescently-Labeled Raji Lymphoblastic B Cells

iNK cells expressing a CD19-specific CAR (FMC63-CAR) or iNK cells without the CAR were engineered to express a CD16 variant construct—a low affinity CD16 variant (iPSC16) or a high affinity CD16 variant (iPSC17 or iPSC18). In an ADCC assay, the resulting iNK cells were tested for killing of either parental Raji or modified RajiACD19 target cells. The parental Raji cells are a lymphoblastic B cell line, which expresses B cell antigens CD19 and CD20. The modified RajiACD19 cells have been modified via CRISPR gene editing to knockout the gene encoding CD19. To trigger ADCC, an anti-CD20 therapeutic antibody rituximab (right bars with circle at top) was included at various concentrations. An IgG1 isotype control was included as a control (left bars with square at top).

The engineered iNK cells were co-cultured with either CellTrace Violet (CTV) labeled Raji cells or CTV labeled RajiACD19 target cells at an E:T of 3:1 in the presence of different concentrations of rituximab or a host-isotype (human IgG1) control (10, 1, 0.1, 0.01 and 0 g/mL Rituximab or host-matched isotype) for 3 hours. The percentage of CTV-positive and non-viable or dead (7-AAD positive) cells were measured using a BD Symphony cytometer. The percentages of CTV+/7-AAD+ cells were graphed by rituximab or isotype control concentration.

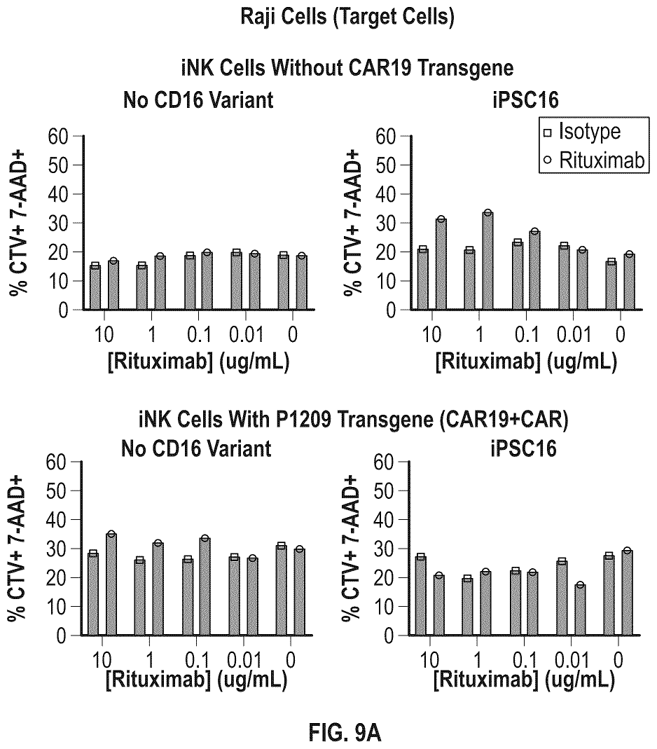

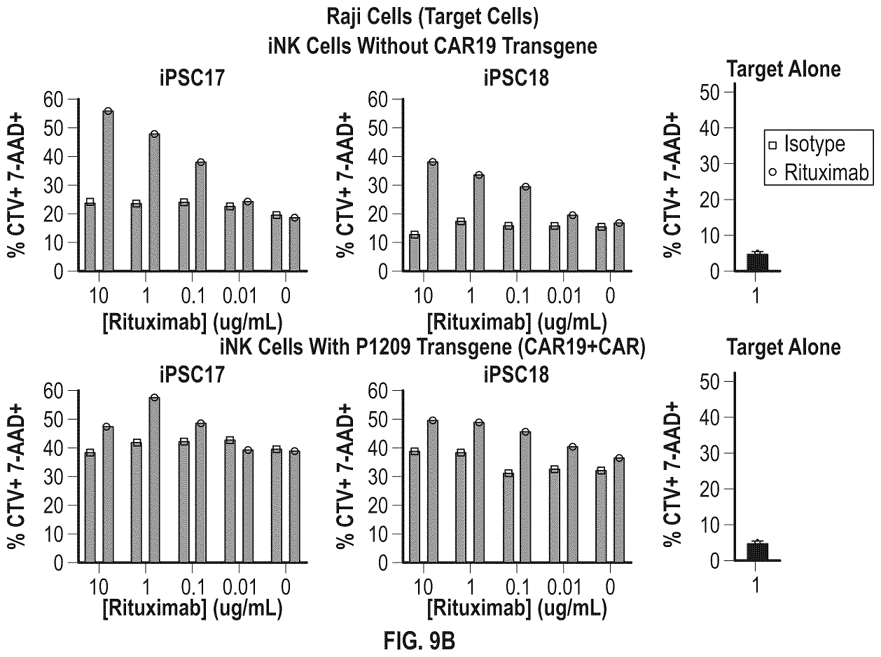

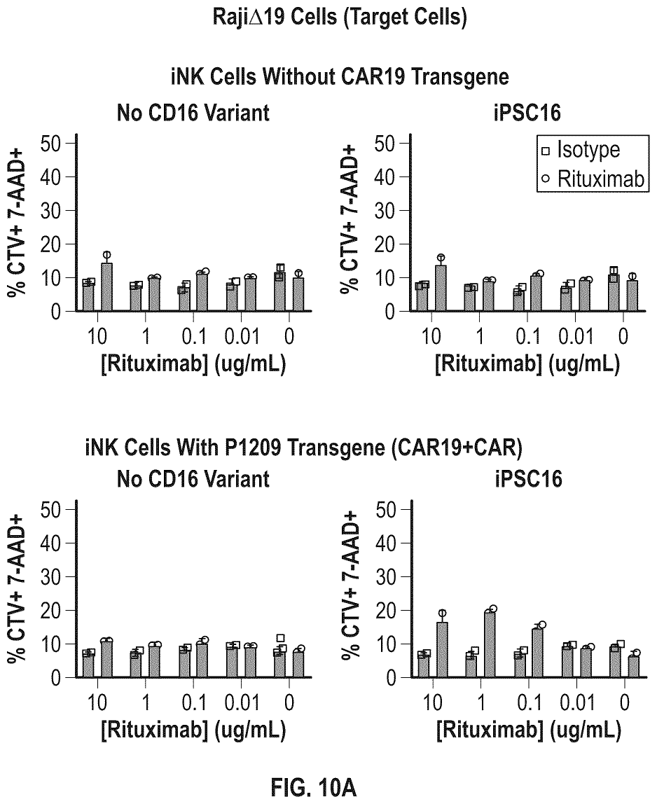

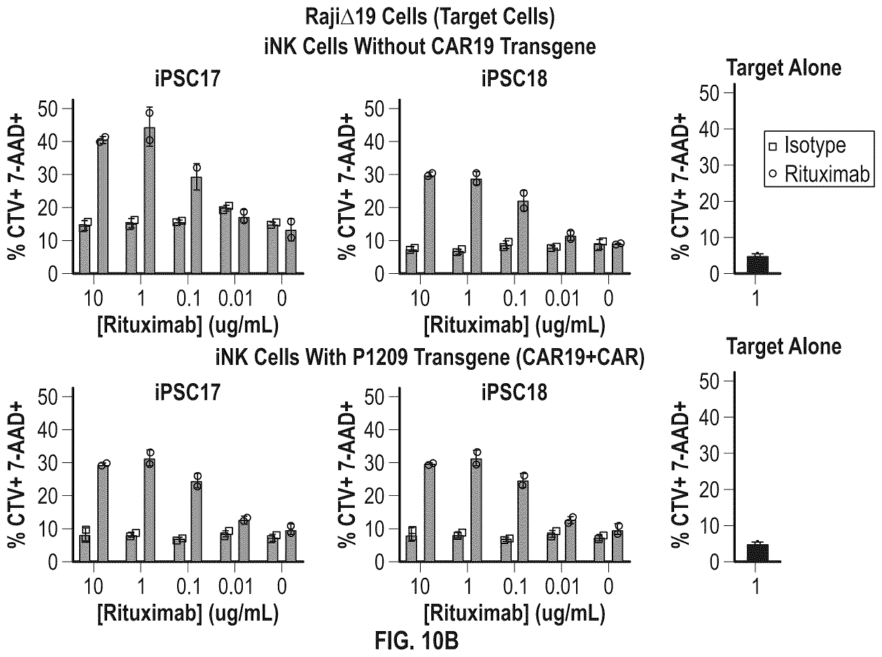

ADCC was seen in iNK cells expressing the low affinity CD16 variant only when rituximab was included. Rituximab enabled iNK cells expressing high affinity CD16 variants were able to kill Raji cells and RajiACD19 cells to a greater extent (FIGS. 9-10).

While various embodiments of the invention(s) of the present disclosure have been shown and described herein, it will be obvious to those skilled in the art that such embodiments are provided by way of example only. Numerous variations, changes, and substitutions may occur to those skilled in the art without departing from the invention(s). It should be understood that various alternatives to the embodiments of the invention(s) described herein may be employed in practicing any one of the inventions(s) set forth herein.

The detailed descriptions above are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

What is claimed is:

1. An induced pluripotent stem cell (iPSC) or a derivative cell thereof comprising an exogenous polynucleotide encoding a CD16 protein and an NKG2D protein, wherein the CD16 protein and the NKG2D protein are operably linked by an autoprotease peptide.

2. The iPSC or the derivative cell thereof of claim 1, wherein the CD16 protein is a CD16 variant protein.

3. The iPSC or the derivative cell thereof of claim 1 or 2, wherein the CD16 variant protein is a high affinity CD16 variant.

4. The iPSC or the derivative cell thereof of any one of claims 1-3, wherein the CD16 variant protein is a non-cleavable CD16 variant.

5. The iPSC or the derivative cell thereof any one of claims 1-4, wherein the CD16 variant protein comprises one or more amino acid substitutions selected from the group consisting of F158V, F176V, S197P, D205A, S219A, T220A, and any combination thereof.

6. The iPSC or the derivative cell thereof of any one of claims 1-5, wherein the CD16 variant protein comprises an amino acid sequence having at least 90% sequence identity to any one of SEQ ID NOS:2 and 5.

7. The iPSC or the derivative cell thereof of any one of claims 1-6, wherein the NKG2D protein is a wildtype NKG2D protein.

8. The iPSC or the derivative cell thereof of any one of claims 1-6, wherein the NKG2D protein comprises an amino acid sequence having at least 90% sequence identity to SEQ ID NO:4.

9. The iPSC or the derivative cell thereof of any one of claims 1-8, wherein the autoprotease peptide is selected from the group consisting of a porcine tesehovirus-1 2A (P2A) peptide, a foot-and-mouth disease virus 2A (F2A) peptide, an Equine Rhinitis A Virus (ERAV) 2A (E2A) peptide, a Thosea asigna virus 2A (T2A) peptide, a cytoplasmic polyhedrosis virus 2A (BmCPV2A) peptide, and a Flacherie Virus 2A (BmIFV2A) peptide.

10. The iPSC or the derivative cell thereof of any one of claims 1-9, wherein the autoprotease peptide is a P2A peptide comprising an amino acid sequence having at least 90% sequence identity to SEQ ID NO:3.

11. The iPSC or the derivative cell thereof of any one of claims 1-10, wherein the exogenous polynucleotide encoding the CD16 protein and the NKG2D protein comprises a nucleic acid sequence having at least 90% sequence identity to SEQ ID NO:6.

12. The iPSC or the derivative cell thereof of any one of claims 1-11, wherein the exogenous polynucleotide is integrated into a gene locus selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus.

13. The iPSC or the derivative cell thereof of any one of claims 1-12, wherein the exogenous polynucleotide is integrated into a gene locus selected from the group consisting of an AAVS1 locus, a B2M locus, a CIITA locus, a CD70 locus, a CLYBL locus, an NKG2A locus, an NKG2D locus, a TAP1 locus, a TAP2 locus, a TAPBP locus, a TRAC locus, a TRBC1 locus, a RFXANK locus, a RFX5 locus, and a RFXAP locus, thereby disrupting expression of the gene.

14. The iPSC or the derivative cell thereof of any one of claims 1-13, wherein the exogenous polynucleotide is integrated into a gene locus selected from the group consisting of an AAVS1 locus, a B2M locus, a CIITA locus, a CD70 locus, a CLYBL locus, an NKG2A locus and a TRAC locus, thereby disrupting expression of the gene.

15. The iPSC or the derivative cell thereof of claim 13 or 14, wherein the disruption of the gene comprises an elimination of or reduced expression of the gene.

16. The iPSC or the derivative cell thereof of any one of claims 12-14, wherein the integration into the gene locus is generated by targeted genome editing.

17. The iPSC or the derivative cell thereof of claim 16, wherein the targeted genome editing comprises using a method selected from the group consisting of a CRISPR method, a zinc finger nuclease method, a TALEN method, a homing nuclease method, a homology recombination method, and any functional variation thereof.

18. The iPSC or the derivative cell thereof of any one of claims 1-17, further comprising a disruption of one or more genes selected from the group consisting of an AAVS1 gene, a B2M gene, a CIITA gene, a CD70 gene, a CLYBL gene, an NKG2A gene, an NKG2D gene, a TAP1 gene, a TAP2 gene, a TAPBP gene, a TRAC gene, a TRBC1 gene, a RFXANK gene, a RFX5 gene, a RFXAP gene, and any combination thereof.

19. The iPSC or the derivative cell thereof of claim 18, wherein the disruption is of the B2M gene and the CIITA gene.

20. The iPSC or the derivative cell thereof of claim 18 or 19, wherein the disruption of the one or more genes comprises an elimination of or reduced expression of the one or more genes.

21. The iPSC or the derivative cell thereof of any one of claims 18-20, wherein the disruption of the one or more genes is generated by targeted genome editing.

22. The iPSC or the derivative cell thereof of claim 21, wherein the targeted genome editing comprises using a method selected from the group consisting of a CRISPR method, a zinc finger nuclease method, a TALEN method, a homing nuclease method, a homology recombination method, and any functional variation thereof.

23. The iPSC or the derivative cell thereof of any one of claims 1-22, further comprising a second exogenous polynucleotide encoding an IL-15 protein.

24. The iPSC or the derivative cell thereof of claim 23, wherein the IL-15 protein comprises an amino acid sequence having at least 90% sequence identity to SEQ ID NO:16.

25. The iPSC or the derivative cell thereof of any one of claims 1-22, further comprising a second exogenous polynucleotide encoding a fusion polypeptide comprising an IL-15 and an IL-15 receptor alpha (IL-15Rα).

26. The iPSC or the derivative cell thereof of claim 25, wherein the fusion polypeptide comprises an amino acid sequence having at least 90% sequence identity to SEQ ID NO:17.

27. The iPSC or the derivative cell thereof of claim 25 or 26, wherein the fusion polypeptide comprises the amino acid sequence of SEQ ID NO:17.

28. The iPSC or the derivative cell thereof of any one of claims 1-27, further comprising a third exogenous polynucleotide encoding a human leukocyte antigen E (HLA-E) protein.

29. The iPSC or the derivative cell thereof of claim 27, wherein the HLA-E comprises an amino acid sequence having at least 90% sequence identity to SEQ ID NO:14.

30. The iPSC or the derivative cell thereof of any one of claims 1-29, further comprising a fourth exogenous polynucleotide encoding a human leukocyte antigen G (HLA-G) protein.

31. The iPSC or the derivative cell thereof of claim 30, wherein the HLA-G comprises an amino acid sequence having at least 90% sequence identity to SEQ ID NO:15.

32. The iPSC or the derivative cell thereof of claim 30 or 31, wherein the HLA-E protein and the HLA-G protein are operably linked by a second autoprotease peptide.

33. The iPSC or the derivative cell thereof of claim 32, wherein the second autoprotease peptide is selected from the group consisting of a P2A peptide, an F2A peptide, an E2A peptide, a T2A peptide, a BmCPV2A peptide and a BmIFV2A peptide.

34. The iPSC or the derivative cell thereof of any one of claims 23-33, wherein the second, third, and/or fourth exogenous polynucleotides are integrated into one or more gene loci selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus, and any combination thereof.

35. The iPSC or the derivative cell thereof of any one of claims 23-34, wherein the second, third, and/or fourth exogenous polynucleotides are integrated into one or more gene loci selected from the group consisting of an AAVS1 locus, a B2M locus, a CIITA locus, a CD70 locus, a CLYBL locus, an NKG2A locus, an NKG2D locus, a TAP1 locus, a TAP2 locus, a TAPBP locus, a TRAC locus, a TRBC1 locus, a RFXANK locus, a RFX5 locus, a RFXAP locus, and any combination thereof.

36. The iPSC or the derivative cell thereof of any one of claims 23-35, wherein the second, third, and/or fourth exogenous polynucleotides are integrated into one or more gene loci selected from the group consisting of an AAVS1 locus, a B2M locus, a CIITA locus, a CD70 locus, a CLYBL locus, an NKG2A locus, an NKG2D locus, a TAP1 locus, a TAP2 locus, a TAPBP locus, a TRAC locus, a TRBC1 locus, a RFXANK locus, a RFX5 locus, a RFXAP locus, and any combination thereof, thereby disrupting the one or more genes.

37. The iPSC or the derivative cell thereof of any one of claims 23-36, wherein the second, third, and/or fourth exogenous polynucleotides are integrated into one or more gene loci selected from the group consisting of an AAVS1 locus, a B2M locus, a CIITA locus, a CD70 locus, a CLYBL locus, an NKG2A locus, a TRAC locus, and any combination thereof, thereby disrupting the one or more genes.

38. The iPSC or the derivative cell thereof of claim 36 or 37, wherein the disruption in the one or more genes comprises an elimination or reduced expression of the one or more genes.

39. The iPSC or the derivative cell thereof of any one of claims 1-38, wherein the iPSC is reprogrammed from whole peripheral blood mononuclear cells (PBMCs).

40. The iPSC or the derivative cell thereof of any one of claims 1-39, wherein the iPSC is derived from a reprogrammed NK or T cell.

41. The iPSC or the derivative cell thereof of any one of claims 1-40, further comprising a fifth exogenous polynucleotide encoding a chimeric antigen receptor (CAR) that binds a target antigen.

42. The iPSC or the derivative cell thereof of claim 41, wherein the target antigen is selected from the group consisting of 17-1A antigen, A3, A33 antigen, AFP, B7H4, Ba 733, BCMA, BrE3 antigen, CA125, CA9 (CAIX), CD1, CD1a, CD3, CD5, CD15, CD16, CD19, CD20, CD21, CD22, CD22, CD23, CD25, CD30, CD33, CD33, CD38, CD45, CD70, CD74, CD79, CD79a, CD80, CD123, CD133, CD138, CEACAM5, CEACAM6, CLDN18.2, CLL1, cMET, colon-specific antigen-p (CSAp), ED-B fibronectin, EGFR, EGFRvIII, EGP-1, EGP-2, EpCAM, EphA1, EphA2, EphA3, EphA4, EphA5, EphA6, EphA7, EphA8, EphA10, EphB1, EphB2, EphB3, EphB4, EphB6, FGFR1, FGFR3, Flt-1, Flt-3, FOLR1, FOLR2, FOLR3, FSHR, GD2, GPC-3, GPRC5D, HCG, a HCG subunit, HER2, HIF-I, HLA-DR, Ia, IGF-I, IL13Rα2, IL-2, IL-6, IL-8, KC4 antigen, KS-1 antigen, KS1-4 antigen, Le-Y, MAGE, MET, MIF, MSLN, MUC1, MUC2, MUC3, MUC4, MUC16, NCA66, NCA90, NCA95, Nectin-4, p53, PAP, PDGFRA, PLGF, PSA, PSMA, ROBO1, RS5, S100, SLAM F7, SLITRK6, TAC, TAG-72, tenascin-C, tenascin-R, tenascin-W, tenascin-X, Thomson-Friedenreich antigen, Tn antigen, TRAILR1, TRAILR2, TRAILR3, TRAILR4, VEGF, a tumor necrosis antigen, an angiogenesis antigen, and an oncogene antigen.

43. The iPSC or the derivative cell thereof of claim 41 or 42, wherein the CAR comprises an antigen-binding domain selected from the group consisting of any provided in Tables 1, 2 and 3.

44. The iPSC or the derivative cell thereof of any one of claims 41-43, wherein the CAR comprises:
(i) a signal peptide;
(ii) an extracellular domain comprising a binding domain that specifically binds the target antigen;
(iii) a hinge region;
(iv) a transmembrane domain,
(v) an intracellular signaling domain; and
(vi) one or more co-stimulatory domains.

45. The iPSC or the derivative cell thereof of claim 44, wherein the signal peptide comprises a GMCSFR signal peptide.

46. The iPSC or the derivative cell thereof of claim 44 or 45, wherein the extracellular domain comprises a single chain Fv (scFv) or a VHH domain that specifically binds the target antigen.

47. The iPSC or the derivative cell thereof of any one of claims 44-46, wherein the hinge region comprises a CD28 hinge region.

48. The iPSC or the derivative cell thereof of any one of claims 44-47, wherein the transmembrane domain comprises a CD28 transmembrane domain.

49. The iPSC or the derivative cell thereof of any one of claims 44-48, wherein the intracellular signaling domain comprises a CD3ζ intracellular domain.

50. The iPSC or the derivative cell thereof of any one of claims 44-49, wherein the one or more co-stimulatory domains comprise a CD28 signaling domain.

51. The derivative cell thereof of any one of claims 1-50, wherein the derivative cell is an NK cell or a T cell.

52. The derivative cell thereof of claim 51, wherein the derivative cell is an NK cell.

53. The derivative cell thereof of claim 51, wherein the derivative cell is a T cell.

54. The derivative cell thereof of any one of claims 1-53, wherein the derivative cell is a CD34+ hematopoietic progenitor cell.

55. A composition comprising a population of the iPSCs or the derivative cells thereof of any one of claims 1-54.

56. An engineered cell comprising:
(i) a first exogenous polynucleotide encoding a CD16 protein and an NKG2D protein, wherein the CD16 protein and the NKG2D protein are operably linked by an autoprotease peptide;
(ii) a second exogenous polynucleotide encoding an exogenous polypeptide comprising an IL-15 protein; and
(iii) optionally, a third exogenous polynucleotide encoding a human leukocyte antigen E (HLA-E) and/or a fourth exogenous polynucleotide encoding a human leukocyte antigen G (HLA-G).

57. The engineered cell thereof of claim 56, further comprising a fifth polynucleotide encoding a combined artificial cell death/reporter system polypeptide comprising an intracellular domain having a herpes simplex virus thymidine kinase (HSV-TK) and a linker, a transmembrane region, and an extracellular domain comprising a prostate-specific membrane antigen (PSMA) extracellular domain or fragment thereof.

58. The engineered cell thereof of claim 56 or 57, wherein the HSV-TK comprises an amino acid sequence having at least 90%, 91%, 92%, 93%, 94%, 95%, 96%, 97%, 98%, 99% or 100% sequence identity to SEQ ID NO:23 or 29.

59. The engineered cell thereof of any one of claims 56-58, wherein the combined artificial cell death/reporter system polypeptide comprises the HSV-TK fused to a truncated variant PSMA polypeptide via the linker.

60. The engineered cell thereof of any one of claims 56-59, wherein the truncated variant PSMA polypeptide comprises an amino acid sequence having at least 90%, 91%, 92%, 93%, 94%, 95%, 96%, 97%, 98%, 99% or 100% sequence identity to SEQ ID NO:24.

61. The engineered cell thereof of any one of claims 56-59, wherein the linker comprises an autoprotease peptide sequence selected from the group consisting of P2A peptide sequence, T2A peptide sequence, E2A peptide sequence, and F2A peptide sequence.

62. The engineered cell thereof of any one of claims 56-60, wherein the artificial cell death/reporter system polypeptide comprises an amino acid sequence having at least 90%, 91%, 92%, 93%, 94%, 95%, 96%, 97%, 98%, 99% or 100% sequence identity to a sequence selected from the group consisting of SEQ ID NO:25.

63. The engineered cell thereof of any one of claims 56-62, wherein the artificial cell death/reporter system polypeptide comprises an amino acid sequence having at least 90%, 91%, 92%, 93%, 94%, 95%, 96%, 97%, 98%, 99% or 100% sequence identity to a sequence selected from the group consisting of SEQ ID NOS:27, 30 and 31.

64. The engineered cell thereof of any one of claims 56-63, wherein the artificial cell death/reporter system polypeptide comprises nucleic acid sequence having at least 90%, 91%, 92%, 93%, 94%, 95%, 96%, 97%, 98%, 99% or 100% sequence identity to a sequence selected from the group consisting of SEQ ID NOS:26, 28 and 32.

65. An engineered cell comprising:
(i) a first exogenous polynucleotide encoding a CD16 protein and an NKG2D protein, wherein the CD16 protein and the NKG2D protein are operably linked by an autoprotease peptide;
(ii) a second exogenous polynucleotide encoding a fusion polypeptide comprising an IL-15 protein and an IL-15 receptor alpha (IL-15Rα) protein; and
(iii) optionally, a third exogenous polynucleotide encoding a human leukocyte antigen E (HLA-E) protein and/or a fourth exogenous polynucleotide encoding a human leukocyte antigen G (HLA-G) protein.

66. The engineered cell of any one of claims 56-65, wherein the engineered cell is an engineered induced pluripotent stem cell (iPSC), an engineered natural killer (NK) cell or an engineered T cell.

67. The engineered cell of any one of claims 56-66, wherein the first exogenous polynucleotide comprises the nucleic acid sequence of SEQ ID NO:6.

68. The engineered cell of any one of claims 56-67, wherein the IL-15 protein comprises an amino acid sequence of SEQ ID NO:16.

69. The engineered cell of any one of claims 56-67, wherein the second exogenous polynucleotide comprises the nucleic acid sequence encoding an IL-15/IL-15Rα fusion protein of SEQ ID NO:17.

70. The engineered cell of any one of claims 56-69, wherein the third exogenous polynucleotide comprises the nucleic acid sequence of SEQ ID NO:21 and the fourth exogenous polynucleotide comprises the nucleic acid sequence of SEQ ID NO:22.

71. The engineered cell of any one of claims 56-69, wherein the HLA-E protein and HLA-G protein are linked by an autoprotease peptide.

72. The engineered cell of any one of claims 56-70, further comprising a disruption of the B2M and CIITA genes.

73. The engineered cell of claim 72, wherein the disruption of the B2M and CIITA genes is generated by targeted genome editing.

74. The engineered cell of 73, wherein the targeted genome editing comprises using a method selected from the group consisting of a CRISPR method, a zinc finger nuclease method, a TALEN method, a homing nuclease method, a homology recombination method, and any functional variation thereof.

75. The engineered cell of any one of claims 56-74, wherein the first exogenous polynucleotide is integrated into a gene locus selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus.

76. The engineered cell of any one of claims 56-75, wherein the second exogenous polynucleotide is integrated into a gene locus selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, a collagen locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus.

77. The engineered cell of any one of claims 56-76, wherein the third exogenous polynucleotide is integrated into a gene locus selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, a collagen locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus.

78. The engineered cell of any one of claims 56-77, wherein the fourth exogenous polynucleotide is integrated into a gene locus selected from the group consisting of an AAVS1 locus, a B2M locus, a CIITA locus, a CCR5 locus, a CD70 locus, a CLYBL locus, an NKG2A locus, an NKG2D locus, a CD38 locus, a TRAC locus, a TRBC1 locus, a ROSA26 locus, a collagen locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus.

79. The engineered cell of any one of claims 56-78, wherein the first exogenous polynucleotide and either the second, third or fourth exogenous polynucleotides are integrated into the B2M gene locus and the CIITA gene locus, thereby disrupting the B2M and CIITA genes.

80. The engineered cell of any one of claims 56-78, wherein the first exogenous polynucleotide is integrated into the CD70 locus and the second exogenous polynucleotide is integrated into the B2M gene locus, thereby disrupting the CD70 and B2M genes.

81. The engineered cell of any one of claims 56-78, wherein the first exogenous polynucleotide is integrated into the CD70 locus and the second exogenous polynucleotide is integrated into the CIITA gene locus, thereby disrupting the CD70 and CIITA genes.

82. The engineered cell of any one of claims 56-78, wherein the first exogenous polynucleotide is integrated into the CD70 locus and the third or fourth exogenous polynucleotide is integrated into the B2M gene locus, thereby disrupting the CD70 and B2M genes.

83. The engineered cell of any one of claims 56-78, wherein the first exogenous polynucleotide is integrated into the CD70 locus and the third or fourth exogenous polynucleotide is integrated into the CIITA gene locus, thereby disrupting the CD70 and CIITA genes.

84. The engineered cell of any one of claims 80-83, wherein the integration into the CD70 locus is into exon 1 of the CD70 gene.

85. The engineered cell of any one of claims 75-84, wherein the integration into the gene locus is generated by targeted genome editing.

86. The engineered cell of claim 85, wherein the targeted genome editing comprises using a method selected from the group consisting of a CRISPR method, a zinc finger nuclease method, a TALEN method, a homing nuclease method, a homology recombination method, and any functional variation thereof.

87. The engineered cell of any one of claims 78-86, further comprising a fifth exogenous polynucleotide encoding a chimeric antigen receptor (CAR) that binds a target antigen.

88. The engineered cell of claim 87, wherein the target antigen is selected from the group consisting of 17-1A antigen, A3, A33 antigen, AFP, B7H4, Ba 733, BCMA, BrE3 antigen, CA125, CA9 (CAIX), CD1, CD1a, CD3, CD5, CD15, CD16, CD19, CD20, CD21, CD22, CD22, CD23, CD25, CD30, CD33, CD33, CD38, CD45, CD70, CD74, CD79, CD79a, CD80, CD123, CD133, CD138, CEACAM5, CEACAM6, CLDN18.2, CLL1, cMET, colon-specific antigen-p (CSAp), ED-B fibronectin, EGFR, EGFRvIII, EGP-1, EGP-2, EpCAM, EphA1, EphA2, EphA3, EphA4, EphA5, EphA6, EphA7, EphA8, EphA10, EphB1, EphB2, EphB3, EphB4, EphB6, FGFR1, FGFR3, Flt-1, Flt-3, FOLR1, FOLR2, FOLR3, FSHR, GD2, GPC-3, GPRC5D, HCG, a HCG subunit, HER2, HIF-I, HLA-DR, Ia, IGF-I, IL13Rα2, IL-2, IL-6, IL-8, KC4 antigen, KS-1 antigen, KS1-4 antigen, Le-Y, MAGE, MET, MIF, MSLN, MUC1, MUC2, MUC3, MUC4, MUC16, NCA66, NCA90, NCA95, Nectin-4, p53, PAP, PDGFRA, PLGF, PSA, PSMA, ROBO1, RS5, S100, SLAM F7, SLITRK6, TAC, TAG-72, tenascin-C, tenascin-R, tenascin-W, tenascin-X, Thomson-Friedenreich antigen, Tn antigen, TRAILR1, TRAILR2, TRAILR3, TRAILR4, VEGF, a tumor necrosis antigen, an angiogenesis antigen, and an oncogene antigen.

89. The engineered cell of claim 88, wherein the CAR comprises an antigen-binding domain selected from the group consisting of any provided in Tables 1, 2 and 3.

90. The engineered cell of any one of claims 87-89, wherein the CAR comprises:
(i) a signal peptide;
(ii) an extracellular domain comprising a binding domain that specifically binds the target antigen;
(iii) a hinge region;
(iv) a transmembrane domain;
(v) an intracellular signaling domain; and
(vi) one or more co-stimulatory domains.

91. The engineered cell of claim 90, wherein the signal peptide comprises a GMCSFR signal peptide.

92. The engineered cell of claim 90 or 91, wherein the extracellular domain comprises a single chain Fv (scFv) or a VHH domain that specifically binds the target antigen.

93. The engineered cell of any one of claims 90-92, wherein the hinge region comprises a CD28 hinge region.

94. The engineered cell of any one of claims 90-93, wherein the transmembrane domain comprises a CD28 transmembrane domain.

95. The engineered cell of any one of claims 90-94, wherein the intracellular signaling domain comprises a CD3ζ intracellular domain.

96. The engineered cell of any one of claims 90-95, wherein the one or more co-stimulatory domains comprise a CD28 signaling domain.

97. The engineered cell of any one of claims 56-96, wherein the engineered iPSC is differentiated into an engineered differentiated cell.

98. The engineered cell of any one of claims 56-97, wherein the engineered iPSC is differentiated into an engineered NK cell.

99. The engineered cell of any one of claims 56-97, wherein the engineered iPSC is differentiated into an engineered T cell.

100. The engineered cell of any one of claims 56-97, wherein the engineered iPSC is differentiated into an engineered CD34+ hematopoietic progenitor cell.

101. A composition comprising a population of the engineered iPSCs of any one of claims 56-96.

102. A composition comprising a population of the engineered differentiated cells of any one of claims 56-101.

103. A composition comprising a population of the engineered NK cells of any one of claims 56-98.

104. A composition comprising a population of the engineered T cells of any one of claims 56-97 and 99.

105. A composition comprising a population of the engineered CD34+ hematopoietic progenitor cells of any one of claims 56-97 and 100.

106. A method of treating cancer in a subject in need thereof, comprising administering the derivative cells thereof of any one of claims 1-54, the engineered NK cells of any one of claims 56-98, the engineered T cells of any one of claims 56-97 and 99, the engineered CD34+ hematopoietic progenitor cells of any one of claims 56-97 and 100, or the composition of any one of claims 55 and 101-105 to the subject in need thereof.

107. The method of claim 106, wherein the cancer is selected from the group consisting of acute lymphocytic leukemia (ALL), acute myeloid leukemia (AML), adenomas, benign lesions, bladder cancers, bone cancers, breast cancers, cancers of the thyroid gland, carcinomas of the larynx, carcinomas of the lung, carcinomas of the mouth, carcinomas of the throat, cervical cancers, chronic lymphocytic leukemia (CLL), chronic myeloid leukemias (CMIL), cutaneous melanomas, endocrine cancers, endometrial cancers, gastrointestinal cancers, genitourinary cancers, glioblastomas, head and neck cancers, hematologic malignancy, hematopoietic cancers, Hodgkin's lymphoma, intraocular melanomas, leukemias, liver cancers, lymphomas, melanomas, myelomas, myeloproliferative disorders, nervous system cancers, non-Hodgkin's lymphoma, ovarian cancers, pancreatic cancers, papillomas, parathyroid gland cancers, prostate cancers, renal cell carcinomas, sarcomas, skin cancers, solid tissue carcinomas, squamous cell carcinomas, and uterine cancers.

108. A method of differentiating the iPSC cell into an NK cell, comprising subjecting the iPSC cell of any one of claims 1-50 and 56-96 to a differentiation protocol comprising culturing the cell in a medium comprising a recombinant human IL-12 protein for the final 24 hours of culturing under the differentiation protocol, thereby generating the NK cell.

109. The method of claim 108, wherein the recombinant human IL-12 protein comprises an amino acid sequence having at least 90%, 91%, 92%, 93%, 94%, 95%, 96%, 97%, 98%, 99% or 100% sequence identity to SEQ ID NO:33.

110. A method of differentiating the iPSC cell into a T cell, comprising subjecting the iPSC cell of any one of claims 1-50 and 56-96 to a differentiation protocol comprising culturing the cell in a medium comprising a recombinant DLL4 variant polypeptide having an amino acid sequence selected from the group consisting of SEQ ID NOS:35-37, thereby generating the T cell.

111. A recombinant DLL4 variant polypeptide having an amino acid sequence selected from the group consisting of SEQ ID NOS:35-37.

112. A method of differentiating the iPSC cell into a CD34+ hematopoietic progenitor cell, comprising subjecting the iPSC cell of any one of claims 1-50 and 56-96 to a differentiation protocol comprising culturing the cell in a pre-selected medium, thereby generating the CD34+ hematopoietic progenitor cell.

113. A polynucleotide encoding a CD16 protein and an NKG2D protein, wherein the CD16 protein and the NKG2D protein are operably linked by an autoprotease peptide.

114. The polynucleotide of claim 113, wherein the CD16 protein comprises an amino acid sequence having at least 90% sequence identity to SEQ ID NO:5.

115. The polynucleotide of claim 113 or 114, wherein the CD16 protein is encoded by a nucleic acid sequence having at least 90% sequence identity to SEQ ID NO:7.

116. The polynucleotide of claim 113, wherein the CD16 protein is a CD16 variant protein.

117. The polynucleotide of claim 115, wherein the CD16 variant protein comprises one or more amino acid substitutions selected from the group consisting of F158V, F176V, S197P, D205A, S219A, T220A, and any combination thereof.

118. The polynucleotide of claim 115 or 117, wherein the CD16 variant comprises an amino acid sequence having at least 90% sequence identity to SEQ ID NO:2 or SEQ ID NO:5.

119. The polynucleotide of any one of claims 115-118, wherein the CD16 variant is encoded by a nucleic acid sequence having at least 90% sequence identity to SEQ ID NO:7.

120. The polynucleotide of any one of claims 115-119, wherein the NKG2D protein comprises an amino acid sequence having at least 90% sequence identity to SEQ ID NO:4.

121. The polynucleotide of any one of claims 115-120, wherein the NKG2D protein is encoded by a nucleic acid sequence having at least 90% sequence identity to SEQ ID NO:9.

122. The polynucleotide of any one of claims 115-120, wherein the NKG2D protein is an NKG2D variant protein.

123. The polynucleotide of claim 121, wherein the NKG2D variant comprises an amino acid sequence having at least 90% sequence identity to SEQ ID NO:4.

124. The polynucleotide of any one of claims 113-123, wherein the autoprotease peptide is selected from the group consisting of a porcine tesehovirus-1 2A (P2A) peptide, a foot-and-mouth disease virus (FMDV) 2A (F2A) peptide, an Equine Rhinitis A Virus (ERAV) 2A (E2A) peptide, a Thosea asigna virus 2A (T2A) peptide, a cytoplasmic polyhedrosis virus 2A (BmCPV2A) peptide, and a Flacherie Virus 2A (BmIFV2A) peptide.

125. The polynucleotide of any one of claims 113-124, wherein the autoprotease peptide is a P2A peptide comprising an amino acid sequence having at least 90% sequence identity to SEQ ID NO:3.

126. The polynucleotide of any one of claims 113-125, wherein the autoprotease peptide is a P2A peptide encoded by a nucleic acid sequence having at least 90% sequence identity to SEQ ID NO:8.

127. The polynucleotide of any one of claims 113-125, wherein the exogenous polynucleotide encoding the CD16 protein and the NKG2D protein comprises the nucleic acid sequence having at least 90% sequence identity to SEQ ID NO:6.

128. The polynucleotide of any one of claims 113-126, wherein the exogenous polynucleotide encoding the CD16 protein and the NKG2D protein has the nucleic acid sequence of SEQ ID NO:6.

129. A vector comprising the polynucleotide of any one of claims 113-127.

130. The vector of claim 128, comprising from 5′ to 3′:
(i) a left homology sequence;
(ii) a promoter;
(iii) the polynucleotide of any one of claims 113-127;
(iv) a terminator and/or a polyadenylation signal sequence; and
(iv) a right homology sequence.

131. The vector of claim 130, wherein the left homology sequence comprises a nucleic acid sequence having at least 90% sequence identity to SEQ ID NO:11.

132. The vector of claim 130 or 131, wherein the right homology sequence comprises a nucleic acid sequence having at least 90% sequence identity to SEQ ID NO:12.

133. The vector of any one of claims 128-132, wherein the vector comprises a nucleic acid sequence having at least 90% sequence identity to SEQ ID NO:13.

134. The vector of any one of claims 128-132, wherein the vector comprises the nucleic acid sequence of SEQ ID NO:13.

135. The vector of any one of claims 128-132, wherein the vector comprises a nucleic acid sequence having at least 90% sequence identity to SEQ ID NO:39.

136. The vector of any one of claims 128-132, wherein the vector comprises the nucleic acid sequence of SEQ ID NO:39.
